(19) United States
(12) Patent Application Publication
(10) Pub. No.: US 2025/0236627 A1
(43) Pub. Date: Jul. 24, 2025

(54) CERTAIN OCTAHYDROFURO[3,4- B]PYRAZINES AS GLP-1 RECEPTOR MODULATORS

(71) Applicant: ASTRAZENECA AB, Sodertalje (SE)

(72) Inventors: Magnus POLLA, Sodertalje (SE); Joakim BERGMAN, Sodertalje (SE); Johan SUNDELL, Sodertalje (SE); Jonas BRÅNALT, Sodertalje (SE); Ekaterina RATKOVA, Sodertalje (SE); Johan KAJANUS, Sodertalje (SE); Magnus JOHANSSON, Sodertalje (SE)

(21) Appl. No.: 18/698,423

(22) PCT Filed: Oct. 4, 2022
(86) PCT No.: PCT/EP2022/077508

§ 371 Date: Apr. 4, 2024

Related U.S. Application Data
(60) Provisional application No. 63/262,109, filed on Oct. 5, 2021.

(51) Int. Cl. C07D 491/048; A61K 31/4985

(52) U.S. Cl. CPC C07D 491/048; A61K 31/4985

(57) ABSTRACT

There are disclosed certain octahydrofuro[3,4-b]pyrazines of formula (1a), and pharmaceutically acceptable salts thereof, together with compositions containing them and their use in therapy. The compounds are GLP-1 receptor modulators and are thereby particularly useful in the treatment or prophylaxis of cardiovascular disease and metabolic conditions, for example Type 2 diabetes.

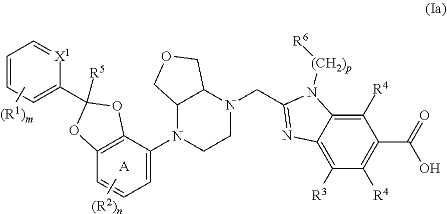

### TECHNICAL FIELD

The technical field relates to certain octahydrofuro[3,4-b]pyrazines, to their use in the treatment of cardiovascular disease and metabolic conditions, for example type 2 diabetes, and to pharmaceutical compositions containing them.

### BACKGROUND

Obesity and type 2 diabetes (T2D) are major and growing health problems worldwide (Lancet, 2014, 9922, 1068-1083). The two diseases are strongly associated with each other, with obesity proceeding development of insulin resistance and T2D. T2D is associated with several comorbidities including cardiovascular disease, renal disease, hypertension, stroke, non-alcoholic fatty liver disease (NAFLD) and non-alcoholic steatohepatitis (NASH) (Lancet, 2005, 9468, 1415-1428).

Incretin hormones including GLP-1 (glucagon-like peptide-1) and GIP (glucose-dependent insulinotropic polypeptide) are gut peptides that are secreted after nutrient intake and stimulate insulin secretion (Diabetes Obes Metab., 2018, 20(Suppl.1), 5-21). GLP-1 secretion from the gut is impaired in obese subjects which may indicate a role in the pathophysiology of obesity (Regulatory Peptides, 2004, 122, 209-217).

GLP-1 is secreted from the L-cells in the lower gut in response to food intake. GLP-1 stimulates insulin secretion from the pancreatic β-cells, in a glucose dependent manner (Diabetologia, 1993, 36, 741-744). GLP-1 also inhibits glucagon secretion, reduces appetite and slows down gastric emptying. The GLP-1 receptor is also present in the heart, kidneys and immune system and activation has been shown to reduce blood pressure, increase natriuresis and decrease inflammation.

GLP-1 is a 37-amino acid peptide, post-translationally processed from pro-glucagon, a 158 amino acid precursor polypeptide (www.uniprot.org, pro-glucagon entry P01275). Several other peptides are also derived from proglucagon and processed in a tissue specific manor, including glucagon and oxyntomodulin. GLP-1 has very short half-life in vivo as it is rapidly degraded by dipeptidyl peptidase-4 (DPP-IV) (Front. Endocrinol. 2019, 10, Article 260, 1-10).

Incretin-based glucose- and body weight-lowering medications include GLP-1 receptor agonists, DPP-IV inhibitors and more recently also combinations of GLP-1 agonists and glucose-dependent insulinotropic polypeptide (GIP) agonists (Peptides, 2020, 125, Article 170202). Traditionally GLP-1 analogues are peptide hormones which have been modified to minimize DPP-IV cleavage and are administered as injectables. The first oral GLP-1 peptide was recently approved but bioavailability is low and the drug needs to be administered in the fasting state, 30 min before nutrient intake which may limit patient compliance (JAMA, 2017, 318(15), 1460-1470). The injectable peptides show increased efficacy over the oral peptides but are limited by the route of administration. Small molecule GLP-1 receptor agonists are in development from several companies and are expected to provide a therapeutic benefit versus peptide based therapies due to early use in the treatment paradigm.

Pharmacological stimulation of GLP-1 receptors has been shown to significantly reduce HbA1c levels, provide long term weight loss and reduce blood pressure. GLP-1 receptor agonists have also been shown to reduce cardiovascular events and prolong life in high-risk patients with T2D and are therefore recommended by the European Association for the Study of Diabetes (EASD) and American Diabetes Association (ADA) in patients with multiple risk factors of cardiovascular disease (CVD) independent of the patients glycemic control (Diabetes Care, 2020, 43, 487-493).

There remains a need for an easily-administered prevention and/or treatment for cardiometabolic and associated diseases.

WO2018/109607 discloses 6-carboxylic acids of benzimidazoles and 4-aza-, 5-aza-, 7-aza- and 4,7-diazabenzimidazoles as GLP-1 receptor agonists, processes to make said compounds, and methods comprising administering said compounds to a mammal in need thereof.

WO2019/239319 and WO2019/239371 disclose 6-carboxylic acids of benzimidazoles and 4-aza-, 5-aza- and 7-aza-benzimidazoles as GLP-1 receptor agonists, processes to make said compounds, and methods comprising administering said compounds to a mammal in need thereof.

WO2020/103815 disclose GLP1 receptor agonist compounds and pharmaceutical compositions thereof, for use in e.g. treating type 2 diabetes mellitus, pre-diabetes, obesity, non-alcoholic fatty liver disease, non-alcoholic steatohepatitis and cardiovascular disease.

WO2020/207474 disclose GLP1 receptor agonist compounds and pharmaceutical compositions thereof, for use in e.g. treating type 2 diabetes mellitus, pre-diabetes, obesity, non-alcoholic fatty liver disease, non-alcoholic steatohepatitis and cardiovascular disease.

WO2020/234726 disclose combinations of GLP-1 receptor agonist compounds and pharmaceutical compositions thereof and an acetyl-CoA carboxylase (ACC) inhibitor or a diacylglycerol acyltransferase (DGAT2) inhibitor, or a ketohexokinase (KHK) inhibitor or farnesoid X receptor (FXR) agonist, for use in e.g. treating type 2 diabetes mellitus, pre-diabetes, obesity, non-alcoholic fatty liver disease, non-alcoholic steatohepatitis and related diseases.

WO2020/263695 discloses glucagon-like peptide-1 receptor agonists and therapeutic uses of the compounds to treat type II diabetes mellitus.

WO2021/081207 discloses compounds that bind to and act as agonists or modulators of the glucagon-like peptide-1 receptor (GLP-1R) and act as agonists or modulators of GLP-1R. The disclosure further relates to the use of the compounds for the treatment and/or prevention of diseases and/or conditions by said compounds.

WO2021/018023 discloses compounds for modulating a Glucagon-like peptide-1 (GLP-1) receptor, and a pharmaceutical use thereof.

WO2021/096284 and WO2021/096304 discloses compounds that act as GLP-1 receptor agonists, for use as therapeutic agents for metabolic diseases.

WO2021/112538 discloses compounds which serves as a GLP-1 receptor agonist and may be useful in the prevention or treatment of a disease associated with GLP-1 activity.

WO2021/154796 discloses GLP-1R agonists, and compositions, methods, and kits thereof. Such compounds are generally useful for treating a GLP-1R mediated disease or condition.

WO2021/160127 discloses GLP-1 agonists, pharmaceutical compositions, and methods of use thereof.

WO2021116874 discloses of solid forms of 2-[[4-[(S)-2-(5-chloropyridin-2-yl)-2-methylbenzo[d][1,3]dioxol-4-yl]piperidin-1-yl]methyl]-1-[[(S)-oxetan-2-yl]methyl]-1H-benzo[d]imidazole-6-carboxylic acid, 1,3-dihydroxy-2-(hydroxymethyl)propan-2-amine salt for pharmaceutical use.

CN113493447A discloses a compound that can be used as a GLP-1 receptor agonist. WO2021197464 discloses fused imidazole derivatives, preparation methods and medical use as a therapeutic agent, especially as GLP-1 receptor agonists.

CN113480534A discloses benzimidazole or azabenzimidazole-6-carboxylate compound that can activate GLP-1R downstream signaling pathway.

WO2021154796 discloses compounds as GLP-1R agonists, and compositions, methods, and kits thereof.

WO2021219019 discloses GLP-1 agonists of formula I, including pharmaceutically acceptable salts and solvates thereof, pharmaceutical compositions, and methods of using the same.

WO2021244645 discloses five-membered heteroaromatic imidazole compounds I and their medical use.

WO2021249492 discloses methyl-substituted benzobisoxazole compound and the use thereof in the preparation of drugs for treating related diseases.

CN113816948A discloses fused imidazole derivatives as GLP-1 receptor agonist in the treatment of diabetes.

WO2021254470 discloses preparation of 6-oxo-3,6-dihydropyridine derivative and a pharmaceutical composition containing the derivative, are used as therapeutic agents, in particular as GLP-1 receptors agonist and in the preparation of drugs for the treatment and/or prevention of diabetes.

WO2022007979 discloses a fused imidazole derivative, a preparation method therefor, a pharmaceutical composition containing the derivative, and the use of same as a therapeutic agent, in particular the use thereof as a GLP-1 receptor agonist.

CN113831337A discloses heterocyclic nitrogen compounds as GLP-1 receptor agonist.

WO2022068772 discloses a kind of benzimidazole derivative, its preparation method and application as GLP-1R agonists.

WO2022042691 discloses GLP-1 agonists, including pharmaceutically acceptable salts and solvates thereof, and pharmaceutical compositions including the same.

WO2022040600 discloses compounds that may be used as a glucagon-like peptide-1 receptors (GLP-1R) agonist.

WO2022028572 discloses GLP-1 agonists, including pharmaceutically acceptable salts and solvates thereof, and pharmaceutical compositions including the same.

WO2022031994 discloses compounds and pharmaceutical compositions thereof, for use in, e.g. treating type 2 diabetes mellitus, pre-diabetes, obesity, non-alc. fatty liver disease, non-alc. steatohepatitis, and cardiovascular disease.

CN114591308A discloses piperazine-imidazole containing GLP-1R receptor agonist compounds and application thereof.

WO2022111624 discloses benzimidazole derivatives that are agonists of a glucagon-like peptide-1 receptor (GLP-1R).

WO2022109182 discloses polyheterocyclic benzimidazole compounds and their preparation and use in the treatment of GLP-1R mediated diseases.

CN114478497A discloses a kind of aryl alkyl acid GLP-1 receptor agonist, its preparation method and application in treatment or prevention of GLP-1-mediated diseases and related diseases.

WO2022078380 discloses compounds that are GLP-1 agonists.

WO2022078407 discloses compounds that are GLP-1 agonists.

WO2022078152 discloses a kind of benzimidazolone compounds, their preparation method and application as GLP-1 receptor agonist.

CN114716423A discloses 5,6-dihydro-1,2,4-triazine compounds as GLP-1 receptor agonist.

CN114634510A discloses imidazolopyridine derivatives, which can be used to prepare drugs for treating GLP-1 receptor agonist mediated diseases.

CN114591296A discloses aromatic heterocyclic derivatives as GLP-1R agonists.

WO2022192430 discloses GLP-1R agonists and compositions, methods, and kits thereof.

WO2022192428 discloses GLP-1R agonists and compositions, methods, and kits thereof.

WO2022184849 discloses GLP-1R agonists, uses and pharmaceutical compositions thereof.

CN114907351A discloses tricyclic GLP-1 receptor agonists.

WO2022165076 discloses substituted benzimidazolecarboxylic acids which are GLP-1 receptor modulator compounds.

CN114805336A discloses fused imidazole compounds that are GLP-1 receptor agonists.

CN114763352A discloses benzimidazole derivatives and its application as GLP-1 receptor agonist.

J. Med. Chem. 2022, 65, 12, 8208-8226 discloses A Small-Molecule Oral Agonist of the Human Glucagon-like Peptide-1 Receptor.

Cell Research 2020, (39), 1140-1142 discloses structural insights into the activation of GLP-1R by a small molecule agonist.

An object is to provide novel GLP-1 receptor modulators useful in therapy. A further object is to provide novel compounds having improved safety profile, e.g with regards to selectivity for the GLP-1 receptor over e.g. phosphodiesterase 3 (PDE3) and/or having improved metabolic stability in the body.

### SUMMARY

There is provided compounds that are modulators of the glucagon-like peptide-1 (GLP1) receptor, their use as medicaments, pharmaceutical compositions containing them and synthetic routes to their production.

In one embodiment, there is provided a compound of Formula (I).

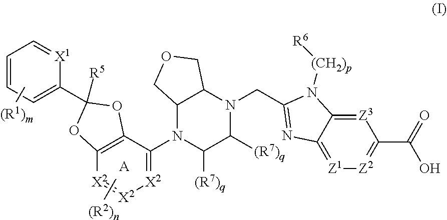

wherein
X¹ is N or C;
X² is independently N or C, provided that no more than two atoms in the aromatic ring A are N;
Z¹ is N or CR³;
Z² and Z³ are each independently N or CR⁴, provided that when Z¹ or Z³ is N, Z² is CR⁴;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
R⁷ is independently selected from F, C₁₋₂alkyl and OC₁₋₂alkyl, wherein said C₁₋₂alkyl is substituted by 0, 1, 2 or 3 substituents independently selected from F;
m is 0, 1, 2 or 3;
n is 0 or 1;
p is 1, 2 or 3;
q is 0, 1 or 2;
or a pharmaceutically acceptable salt thereof.

The compounds of Formula (I) are modulators of the GLP-1 receptor. Thus, the compounds of Formula (I) can be used as a medicament, in particular for disorders, disease or conditions responsive to modulation of the GLP-1 receptor, and more specifically cardiovascular disease and metabolic conditions.

In another embodiment there is provided a compound of Formula (I), or a pharmaceutically acceptable salt of a compound of Formula (I), wherein the stereochemistry is undefined, e.g. a racemate or a mixture of diastereomers.

In another embodiment there is provided a compound of Formula (I), or a pharmaceutically acceptable salt of a compound of Formula (I), wherein the stereochemistry is defined.

In another embodiment there is provided a pharmaceutical formulation comprising a therapeutically effective amount of a compound of Formula (I), or a pharmaceutically acceptable salt of a compound of Formula (I), and a pharmaceutically acceptable diluent, excipient and/or inert carrier.

In a further embodiment there is provided a pharmaceutical formulation comprising a compound of Formula (I), or a pharmaceutically acceptable salt of a compound of Formula (I), for use in the treatment of a condition where modulation of the GLP-1 receptor would be beneficial.

In a further embodiment there is provided a compound of Formula (I), or a pharmaceutically acceptable salt of a compound of Formula (I), for use in therapy, especially in the treatment of cancer in a mammal, particularly a human.

In a further embodiment there is provided the use of a compound of Formula (I), or a pharmaceutically acceptable salt of a compound of Formula (I), for the manufacture of a medicament for the treatment of cardiovascular disease and metabolic conditions.

According to another aspect there is provided a process for the preparation of compounds of Formula (I), or pharmaceutically acceptable salts of compounds of Formula (I), and the intermediates used in the preparation thereof.

The compounds of Formula (I) described herein have the advantage that they may be more efficacious, be less toxic, be more selective, be more potent, produce fewer side effects, be more easily absorbed, and/or have a better pharmacokinetic profile (e.g. higher oral bioavailability and/or lower clearance), than compounds known in the prior art.

### BRIEF DESCRIPTION OF THE DRAWINGS

FIG. 1 shows the molecular structure of Intermediate 170, 4-chlorobenzyl (4aR,7aS)-hexahydrofuro[3,4-b]pyrazine-1(2H)-carboxylate.

### DETAILED DESCRIPTION

This detailed description and its specific examples, while indicating embodiments, are intended for purposes of illustration only. Therefore, there is no limitation to the illustrative embodiments described in this specification. In addition, it is to be appreciated that various features that are, for clarity reasons, described in the context of separate embodiments, also may be combined to form a single embodiment. Conversely, various features that are, for brevity reasons, described in the context of a single embodiment, also may be combined to form sub-combinations thereof.

Listed below are definitions of various terms used in the specification and claims.

It is to be understood that where in this specification a group is qualified by “defined above” the said group encompasses the first occurring and broadest definition as well as each and all of the other definitions for that group.

In this specification, the term “modulator” is used to describe a compound that exhibit varying receptor agonism, either full agonism, or partial agonism.

It is to be understood that in this specification “C₁₋₄” means a carbon group having 1, 2, 3 or 4 carbon atoms.

It is to be understood that in this specification “C₁₋₂” means a carbon group having 1 or 2 carbon atoms.

In this specification, unless stated otherwise, the term “alkyl” includes both straight and branched chain alkyl groups and may be, but is not limited to, methyl, ethyl, n-propyl, i-propyl, n-butyl, sec-butyl, iso-butyl or tert-butyl.

It is to be understood that in this specification “(5- to 6-membered)heteroaryl” means an aromatic ring with 5 to 6 atoms and containing one or more heteroatoms independently selected from nitrogen, oxygen or sulphur.

It is to be understood that in this specification “(6-membered)heteroaryl” means an aromatic ring with 6 atoms and containing one or more heteroatoms independently selected from nitrogen, oxygen or sulphur.

It is to be understood that in this specification “(6-membered)heteroaryl” means for example pyridine.

It is to be understood that in this specification “(5-membered)heteroaryl” means an aromatic ring with 5 atoms and containing one or more heteroatoms independently selected from nitrogen, oxygen or sulphur.

It is to be understood that in this specification “(4- to 6-membered)heterocycloalkyl” means a partially or completely saturated ring system with 4 to 6 atoms and wherein at least one of the ring carbon atoms is replaced with a heteroatom independently selected from nitrogen, oxygen or sulphur.

It is to be understood that in this specification a “heterocycloalkyl” substituent may be attached via a nitrogen atom having the appropriate valences, or via any ring carbon atom.

It is to be understood that in this specification a “heterocycloalkyl” or “heteroaryl” substituent may be further substituted.

In this specification, unless stated otherwise, the term “pharmaceutically acceptable” is used to characterize a moiety (e.g. a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

There is provided compounds of Formula (I) wherein X¹, X², Z¹, Z², Z³, R¹-R⁷, m, n, p and q are as defined in Formula (I).

In one embodiment X¹ is N or C.

In a further embodiment X¹ is N.

In still a further embodiment X¹ is C.

R¹ is 0, 1, 2 or 3 substituents independently selected from F, Cl, Br, CN, OCH₃, OCFH₂, OCF₂H, OCF₃, CH₃, CFH₂, CF₂H and CF₃.

In one embodiment X² is independently N or C, provided that no more than two atoms in the aromatic ring A are N.

In a further embodiment X² is C.

In one embodiment Z¹ is N or CR³.

In a further embodiment Z¹ is N.

In still a further embodiment Z¹ is CR³.

R³ is selected from H, F, Cl, N(CH₃)₂, C₁₋₂alkyl and OC₁₋₂alkyl, wherein said C₁₋₂alkyl is substituted by 0, 1, 2 or 3 F.

In one embodiment Z² and Z³ are each independently N or CR⁴, provided that when Z¹ or Z³ is N, Z² is CR⁴.

In a further embodiment Z¹ and Z² are N.

In still a further embodiment Z¹ and Z³ are N.

In still a further embodiment Z² and Z³ are N.

In still a further embodiment Z¹ is N, Z² and Z³ are CR⁴.

In still a further embodiment Z² is N, Z¹ and Z³ are CR⁴.

In still a further embodiment Z³ is N, Z¹ and Z² are CR⁴.

In still a further embodiment Z¹, Z² and Z³ are CR⁴.

R⁴ is independently selected from H, F, Cl, OH, CH₃, CFH₂, CF₂H, CF₃, OCH₃, OCFH₂, OCF₂H and OCF₃.

In one embodiment R¹ is 0, 1, 2 or 3 substituents independently selected from F, Cl, Br, CN, OCH₃, OCFH₂, OCF₂H, OCF₃, CH₃, CFH₂, CF₂H and CF₃.

In a further embodiment R¹ is 0, 1 or 2 substituents independently selected from F, Cl, Br, CN, OCH₃, OCFH₂, OCF₂H, OCF₃, CH₃, CFH₂, CF₂H and CF₃.

In still a further embodiment R¹ is 0, 1, 2 or 3 substituents independently selected from F, Cl, Br, CN, OCH₃.

In still a further embodiment R¹ is 0, 1 or 2 substituents independently selected from F, Cl, Br, CN, OCH₃.

In still a further embodiment R¹ is 0, 1 or 2 substituents independently selected from F, Cl and CN.

In still a further embodiment R¹ is 0 or 1 substituents selected from F, Cl and CN.

In one embodiment R² is selected from 0 or 1 F, Cl or CN.

In one embodiment R³ is selected from H, F, Cl, N(CH₃)₂, C₁₋₂alkyl and OC₁₋₂alkyl, wherein said C₁₋₂alkyl is substituted by 0, 1, 2 or 3 F.

In a further embodiment R³ is selected from H, F, Cl, C₁₋₂alkyl and OC₁₋₂alkyl, wherein said C₁₋₂alkyl is substituted by 0, 1, 2 or 3 F.

In still a further embodiment R³ is selected from H, F, Cl, CH₃, CFH₂, CF₂H, CF₃, OCH₃, OCFH₂, OCF₂H and OCF₃.

In still a further embodiment R³ is selected from H, F, Cl, CH₃ and OCH₃.

In one embodiment R⁴ is independently selected from H, F, Cl, OH, CH₃, CFH₂, CF₂H, CF₃, OCH₃, OCFH₂, OCF₂H and OCF₃.

In a further embodiment R⁴ is independently selected from H, F, Cl, OH, CH₃ and OCH₃.

In still a further embodiment R⁴ is independently selected from H, F, Cl, CH₃ and OCH₃.

In still a further embodiment R⁴ is independently selected from H, F and Cl.

In one embodiment R⁵ is selected from H, CH₃, CFH₂, CF₂H and CF₃; In a further embodiment R⁵ is selected from H, and CH₃.

In still a further embodiment R⁵ is CH₃.

In one embodiment R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F.

In a further embodiment R⁶ is selected from C₁₋₄alkyl, O(C₁₋₄alkyl) and S(C₁₋₄alkyl), wherein said C₁₋₄alkyl is substituted by 0 or 1 substituent selected from CN or OCH₃, and 0, 1, 2 or 3 F.

In still a further embodiment R⁶ is selected cyclopropyl, cyclobutyl, O(cyclopropyl) or S(cyclopropyl), said cyclopropyl and cyclobutyl is substituted by 0 or 1 substituent selected from CN, OCH₃, OCFH₂, OCF₂H, OCF₃ and CH₂CN and 0, 1, 2 or 3 F.

In still a further embodiment R⁶ is selected from (4- to 6-membered)heterocycloalkyl and (5- to 6-membered)heteroaryl.

In still a further embodiment R⁶ is selected from (5- to 6-membered)heteroaryl.

In still a further embodiment R⁶ is selected from (4- to 6-membered)heterocycloalkyl.

In still a further embodiment R⁶ is oxetan-2-yl.

In one embodiment R⁷ is independently selected from F, C₁₋₂alkyl and OC₁₋₂alkyl, wherein said C₁₋₂alkyl is substituted by 0, 1, 2 or 3 substituents independently selected from F.

In a further embodiment R⁷ is independently selected from F, C₁₋₂alkyl and OC₁₋₂alkyl.

In still a further embodiment R⁷ is independently selected from F, CH₃ and OCH₃.

In one embodiment m is 0, 1, 2 or 3.

In a further embodiment m is 0, 1, or 2.

In still a further embodiment m is 1 or 2

In still a further embodiment m is 0 or 1.

In still a further embodiment m is 1.

In still a further embodiment m is 0.

In one embodiment n is 0 or 1.

In a further embodiment n is 1.

In still a further embodiment n is 0.

In one embodiment p is 1, 2 or 3.

In a further embodiment p is 1 or 2.

In still a further embodiment p is 1.

In one embodiment q is 0, 1 or 2.

In a further embodiment q is 0 or 1.

In still a further embodiment q is 0.

In one embodiment, there is provided a compound of Formula (Ia),

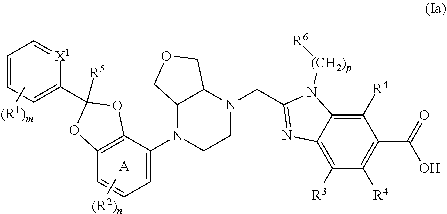

wherein
X¹ is N or C;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1, 2 or 3;
n is 0 or 1;
p is 1, 2 or 3;
or a pharmaceutically acceptable salt thereof.

In a further embodiment, there is provided a compound of Formula (Ia),

wherein
X¹ is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1 or 2;
n is 0 or 1;
p is 1, 2 or 3;
or a pharmaceutically acceptable salt thereof.

In still a further embodiment, there is provided a compound of Formula (Ia),

wherein
X¹ is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl;
m is 0, 1 or 2;
n is 0 or 1;
p is 1;
or a pharmaceutically acceptable salt thereof.

In one embodiment, there is provided a compound of Formula (Ib),

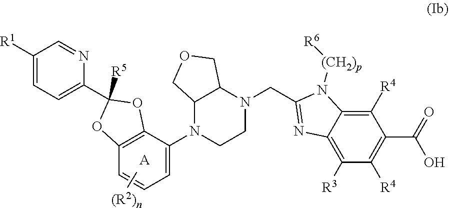

wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In a further embodiment, there is provided a compound of Formula (Ib),

wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
is or a pharmaceutically acceptable salt thereof.

In still a further embodiment, there is provided a compound of Formula (Ib),

wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl;
n is 0 or 1;
p is 1;
or a pharmaceutically acceptable salt thereof.

In one embodiment, there is provided a compound of Formula (Ic),

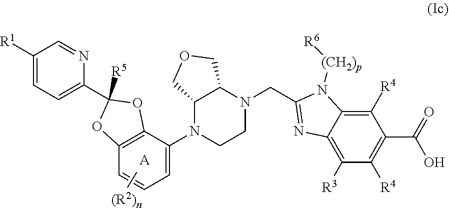

wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In a further embodiment, there is provided a compound of Formula (Ic),

wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In still a further embodiment, there is provided a compound of Formula (Ic),

wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl;
n is 0 or 1;
p is 1;
or a pharmaceutically acceptable salt thereof.

In one embodiment, there is provided a compound of Formula (Id),

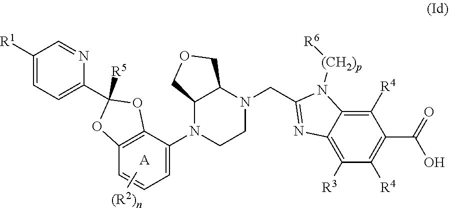

wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In a further embodiment, there is provided a compound of Formula (Id),

wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In still a further embodiment, there is provided a compound of Formula (Id),

wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl;
n is 0 or 1;
p is 1;
or a pharmaceutically acceptable salt thereof.

In one embodiment the compounds of formula (I) are selected from:
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
rel-1-(2-(1H-Pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
rel-1-(2-(1H-Pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
rel-1-(2-(1H-Pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylic acid,
rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid,
4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
4-Chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylic acid,
rel-4-Chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylic acid,
rel-4-Chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylic acid,
rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylic acid,
rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylic acid,
rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid,
rel-4-Chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
rel-4-Chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
rel-1-(2-(1H-Pyrazol-1-yl)ethyl)-4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
2-(((4aS,7aR)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
4-Chloro-2-(((4aR,7aS)-4-((s)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
4-Chloro-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
1-(2-(1H-Pyrazol-1-yl)ethyl)-2-(((4aR,7aS)-4-((S)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid,
1-(2-(1H-Pyrazol-1-yl)ethyl)-2-(((4aS,7aR)-4-((S)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
1-(2-(1H-Pyrazol-1-yl)ethyl)-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
1-(2-(1H-Pyrazol-1-yl)ethyl)-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
1-(2-(1H-Pyrazol-1-yl)ethyl)-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylic acid,
1-(2-(1H-Pyrazol-1-yl)ethyl)-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid,
4-Chloro-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
4-Chloro-2-(((4aS,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
4-Chloro-2-(((4aR,7aS)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
4-Chloro-2-(((4aS,7aR)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylic acid,
4-Chloro-2-(((4aR,7aS)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylic acid,
4-Chloro-2-(((4aS,7aR)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylic acid,
4-Chloro-2-(((4aR,7aS)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylic acid,
4-Chloro-2-(((4aS,7aR)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid,
4-Chloro-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
4-Chloro-2-(((4aS,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
rel-4-Chloro-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-4-Chloro-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
1-(2-(1H-Pyrazol-1-yl)ethyl)-4-chloro-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
1-(2-(1H-Pyrazol-1-yl)ethyl)-4-chloro-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
and pharmaceutically acceptable salts thereof.

It shall be noted that any one of these specific compounds may be disclaimed from any of the herein mentioned embodiments.

In one embodiment there is provided a process for the preparation of compounds of formula (I), or pharmaceutically acceptable salts of compounds of formula (I), and the intermediates used in the preparation thereof.

Another embodiment is a product obtainable by any of the processes or examples disclosed herein.

### Medical and Pharmaceutical Use

The compounds of formula (I) and their pharmaceutically acceptable salts are believed to be useful in the prevention or treatment of cardiovascular disease and metabolic conditions, including but not limited to type 2 diabetes (T2D), obesity, non-alcoholic fatty liver disease (NAFLD) and non-alcoholic steatohepatitis (NASH), in a mammal, particularly a human.

For the avoidance of doubt, as used herein, the term “treatment” includes therapeutic and/or prophylactic treatment.

When a compound or salt described herein is administered as therapy for treating a disorder, a “therapeutically effective amount” is an amount sufficient to reduce or completely alleviate symptoms or other detrimental effects of the disorder, cure the disorder, reverse, completely stop, or slow the progress of the disorder or reduce the risk of the disorder getting worse.

The compounds described herein are thus indicated both in the therapeutic and/or prophylactic treatment of these conditions.

The compounds described herein have the advantage that they may be more efficacious, be less toxic, be more selective, be more potent, produce fewer side effects, be more easily absorbed, and/or have a better pharmacokinetic profile (e.g. higher oral bioavailability and/or lower clearance), than compounds known in the prior art.

For the above-mentioned therapeutic indications, the dosage administered will vary with the compound employed, the mode of administration and the treatment desired. However, in general, satisfactory results are obtained when the compounds are administered at a dosage of the solid form of between 1 mg and 2000 mg per day.

The compounds of formula (I), and pharmaceutically acceptable derivatives thereof, may be used on their own, or in the form of appropriate pharmaceutical compositions in which the compound or derivative is in admixture with a pharmaceutically acceptable adjuvant, diluent or carrier. Thus, another aspect concerns a pharmaceutical composition comprising a novel compound of formula (I), or a pharmaceutically acceptable salt thereof, in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, Pharmaceuticals—The Science of Dosage Form Designs, M. E. Aulton, Churchill Livingstone, 2^{nd} Ed. 2002. In one embodiment the pharmaceutical composition preferably comprises less than 80% and in another embodiment less than 50% of a compound of formula (I), or a pharmaceutically acceptable salt thereof.

In one embodiment there is provided a compound selected from any one of the compounds of formula (I), or a pharmaceutically acceptable salt of a compound of formula (I), for use in therapy, especially in the prevention or treatment of cardiovascular disease and metabolic conditions, including but not limited to type 2 diabetes (T2D), obesity, non-alcoholic fatty liver disease (NAFLD) and non-alcoholic steatohepatitis (NASH).

These and other embodiments are described in greater detail herein below, where further aspects will be apparent to one skilled in the art from reading this specification.

### Combination Therapy

The compounds of formula (I), or pharmaceutically acceptable salts thereof, may also be administered in conjunction with other compounds used for the treatment of the above conditions.

In another embodiment, there is a combination therapy wherein a compound selected from any one of the compounds of formula (I), or a pharmaceutically acceptable salt thereof, and a second active ingredient are administered concurrently, sequentially or in admixture, for the treatment of one or more of the conditions listed above. Such a combination may be used in combination with one or more further active ingredients.

When used in a combination therapy, it is contemplated that the a compound selected from any one of the compounds of formula (I), or pharmaceutically acceptable salts thereof, and the other active ingredients may be administered in a single composition, completely separate compositions, or a combination thereof. It also is contemplated that the active ingredients may be administered concurrently, simultaneously, sequentially, or separately. The particular composition(s) and dosing frequency(ies) of the combination therapy will depend on a variety of factors, including, for example, the route of administration, the condition being treated, the species of the patient, any potential interactions between the active ingredients when combined into a single composition, any interactions between the active ingredients when they are administered to the animal patient, and various other factors known to physicians (in the context of human patients), veterinarians (in the context of non-human patients), and others skilled in the art.

### Pharmaceutical Compositions

There is provided a method of treatment of a condition where modulation of GLP-1 receptor is required, which method comprises administration of a therapeutically effective amount of a compound selected from any one of the compounds of formula (I) to a person suffering from, or susceptible to, such a condition.

The compounds of formula (I) will normally be administered via the oral, topical, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable salt thereof, in a pharmaceutically acceptable dosage form. Depending upon the disorder and patient to be treated and the route of administration, the compositions may be administered at varying doses. Conventional procedures for the selection and preparation of suitable pharmaceutical formulations are described in, for example, Pharmaceuticals—The Science of Dosage Form Designs, M. E. Aulton, Churchill Livingstone, 2^{nd} Ed. 2002.

In one embodiment suitable daily doses of the compounds of formula (I) in therapeutical treatment of humans are about 0.0001-100 mg/kg body weight, in another embodiment about 0.01-10 mg/kg body weight.

The optimum dosage and frequency of administration will depend on the particular condition being treated and its severity; the species of the patient; the age, sex, size and weight, diet, and general physical condition of the particular patient; brain/body weight ratio; other medication the patient may be taking; the route of administration; the formulation; and various other factors known to physicians and others skilled in the art.

According to a further aspect there is thus provided a pharmaceutical formulation comprising a compound selected from any one of the compounds of formula (I), or pharmaceutically acceptable derivatives thereof, in admixture with a pharmaceutically acceptable adjuvant, diluent and/or carrier.

The compound of formula (I) may be present in the pharmaceutical formulation in a concentration from 0.1 to 99.5%, such as from 0.5 to 95%, by weight of the total formulation.

### Preparation of the Compounds

The protection and deprotection of functional groups is described in Protective Groups in Organic Synthesis, 4^{th} Ed, T. W. Greene and P. G. M. Wuts, Wiley-Interscience (2006) and Protecting Groups, 3^{rd} Ed, P. J. Kocienski, Georg Thieme Verlag (2005).

A further embodiment encompasses pharmaceutically acceptable salts of the compounds of formula (I).

A salt of a compound selected from any one of formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound. In some embodiments (particularly where the salt is intended for administration to an animal, e.g. a human, or is a reagent for use in making a compound or salt intended for administration to an animal), the salt is pharmaceutically acceptable.

The term “pharmaceutically acceptable” is used to characterize a moiety (e.g. a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

Where the compound is sufficiently basic, pharmaceutically acceptable salts include, but are not limited to, inorganic or organic acid addition salts.

For reviews on suitable salts, see Berge et al., J. Pharm. Sci., 1977, 66, 1-19 or Handbook of Pharmaceutical Salts: Properties, selection and use, P. H. Stahl, P. G. Vermuth, IUPAC, Wiley-VCH, 2002.

Where an acid co-former is a solid at r.t. and there is no or only partial proton transfer between the compound of formula (I) and such an acid co-former, a co-crystal of the co-former and compound of formula (I) may result rather than a salt. All such co-crystal forms of the compound of formula (I) are encompassed herein.

It is also to be understood that certain compounds of formula (I) may exist in solvated form, e.g. hydrates, including solvates of a pharmaceutically acceptable salt of a compound of formula (I).

In a further embodiment, certain compounds of formula (I) may exist as racemates and racemic mixtures, single enantiomers, individual diastereomers and diastereomeric mixtures. Certain compounds of formula (I) may also contain linkages (e.g. carbon-carbon bonds, carbon-nitrogen bonds such as amide bonds) wherein bond rotation is restricted about that particular linkage, e.g. restriction resulting from the presence of a ring bond or double bond. Stereoisomers may be separated using conventional techniques, e.g. chromatography or fractional crystallization, or the stereoisomers may be made by stereoselective synthesis.

In a further embodiment, the compounds of formula (I) encompass any isotopically-labelled (or “radio-labelled”) derivatives of a compound of formula (I). Such a derivative is a derivative of a compound of formula (I) wherein one or more atoms are replaced by an atom having an atomic mass or mass number different from the atomic mass or mass number typically found in nature. Examples of isotopes that may be incorporated include ²H (also written as “D” for deuterium).

In a further embodiment, the compounds of formula (I) may be administered in the form of a prodrug which is broken down in the human or animal body to give a compound of the formula (I).

Various forms of prodrugs are known in the art. For examples of prodrug derivatives, see: Nature Reviews Drug Discovery 2008, 7, 255 and references cited therein. Intermediate compounds may also exist in enantiomeric forms and may be used as purified enantiomers, diastereomers, racemates or mixtures.

### EXAMPLES

The following examples are non-limiting examples.

### General Conditions

(i) operations were carried out at room temperature (rt), i.e. in the range 17 to 25° C. and under an atmosphere of an inert gas such as N₂ unless otherwise stated;
(ii) where reactions refer to being degassed or purged, this can be performed for example by purging the reaction solvent with a constant flow of nitrogen for a suitable period of time (for example 5 to 10 min) or by repeatedly evacuating the vessel and backfill with appropriate inert atmosphere (for example nitrogen (g) or argon (g));
(iii) where reactions refer to the use of a microwave reactor, one of the following microwave reactors were used: Biotage Initiator, Personal Chemistry Emrys Optimizer, Personal Chemistry Smith Creator or CEM Explorer;
(iv) in general, the course of reactions was followed by thin layer chromatography (TLC) and/or analytical high performance liquid chromatography (HPLC or UPLC) which was usually coupled to a mass spectrometer (LCMS);
(v) when necessary, organic solutions were dried over anhydrous MgSO₄ or Na₂SO₄, or by using ISOLUTE® Phase Separator, and work-up procedures were carried out using traditional phase separating techniques;
(vi) It is understood that washing solutions used in the work-up procedures or reagent used for acidifying such as e.g. Brine, NaHCO₃, NH₄Cl, HCl, NaH₂PO₄ are presumed to be aqueous solutions unless otherwise stated;
(vii), evaporations were carried out either by rotary evaporation in vacuo or in a Genevac HT-4/EZ-2 or Biotage V10;
(viii) unless otherwise stated, flash column chromatography was performed on straight phase silica, using either Merck Silica Gel (Art. 9385) or pre-packed cartridges such as Biotage® SNAP cartridges (40-63 m silica, 4-330 g), Biotage® Sfar Silica HC D cartridges (20 μm, 10-100 g), Interchim puriFlash™ cartridges (25 μm, 4-120 g), Interchim puriFlash™ cartridges (50 μm, 25-330 g), Grace™ GraceResolv™ Silica Flash Cartridges (4-120 g) or Agela Flash Colum Silica-CS cartridges (80-330 g), or on reversed phase silica using Agela Technologies C-18, spherical cartridges (20-35 μm, 100 A, 80-330 g), manually or automated using a Grace Reveleris® X2 Flash system or similar system;
(ix) preparative reverse phase HPLC and preparative reverse phase SFC were performed using standard HPLC and SFC instruments, respectively, equipped with either a MS and/or UV triggered fraction collecting instrument, using either isocratic or a gradient of the mobile phase as described in the experimental section, and one of the following methods as described below;

### HPLC Prep Methods:

PrepMethod A: The compound was purified by preparative HPLC on a Kromasil C8 column (10 μm, 250×20 mm ID) using a gradient of MeCN in H₂O/MeCN/FA (95/5/0.2) as mobile phase.
PrepMethod B: The compound was purified by preparative HPLC on a YMC Triart C18 column (5 μm, 100×20 mm ID) using a gradient of H₂O/MeCN/0.1% NH₄OH as mobile phase.
PrepMethod C: The compound was purified by preparative HPLC on a XBridge™ C18 column (10 μm, 250×19 mm ID) using a gradient of MeCN in H₂O/MeCN/NH₃ (95/5/0.2) as mobile phase.
PrepMethod D: The compound was purified by preparative HPLC on a SunFire column (5 μm, 100×19 mm ID) using a gradient of MeCN in H₂O as mobile phase.
PrepMethod E: The compound was purified by preparative HPLC on a SunFire column (5 μm, 150×30 mm ID) using a gradient of MeCN in 0.15 M TFA (aq) at pH3 as mobile phase.
PrepMethod F: The compound was purified by preparative HPLC on a SunFire C18 ODB column (5 μm, 150×30 mm ID) using a gradient of MeCN in FA (aq) at pH3 as mobile phase.
PrepMethod G: The compound was purified by preparative HPLC on a Chromatorex C18 SMB100-5T column (5 μm, 100×19 mm ID) using a gradient of MeCN in H₂O as mobile phase.
PrepMethod H: The compound was purified by preparative HPLC on a XBridge™ C18 column (10 μm, 250×50 mm ID) using a gradient of MeCN in H₂O/MeCN/NH₃ (95/5/0.2) as mobile phase.

Relevant fractions were collected, combined and freeze-dried to give the purified compound or relevant fractions were collected, combined and concentrated at reduced pressure, extracted with DCM or EtOAc, and the organic phase was dried either over Na₂SO₄ or by using a phase-separator, and then concentrated at reduced pressure to give the purified compound.
(x) chiral preparative chromatography was carried out using HPLC or SFC on a standard HPLC or SFC instruments, respectively, and using either isocratic or gradient run with mobile phase as described in the experimental section;
(xi) yields, where present, are not necessarily the maximum attainable, and when necessary, reactions were repeated if a larger amount of the reaction product was required;
(xii) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;
(xiii) in general, the structures of the end-products of the Formula (I) were confirmed by nuclear magnetic resonance (NMR) and/or mass spectral techniques; proton NMR chemical shift values were measured on the delta scale using Bruker Avance III 300, 400, 500 and 600 spectrometers, operating at ¹H frequencies of 300, 400, 500 and 600 MHz, respectively. The experiments were typically recorded at 25° C. Chemical shifts are given in ppm with the solvent as internal standard. Protons on heteroatoms such as NH and OH protons are only reported when detected in NMR and can therefore be missing. In certain instances, protons can be masked or partially masked by solvent peaks and will therefore either be missing and not reported or reported as multiplets overlapping with solvent. The following abbreviations have been used (and derivatives thereof, e.g. dd, doublet of doublets, etc.): s, singlet; d, doublet; t, triplet; q, quartet; m, multiplet; br, broad; qn, quintet; p, pentet, h, heptet, brs, broad singlet. It is understood, where the NMR spectra contains residual impurities and/or residual solvent(s), this is not reported unless it coincides or partially coincides with peaks of Intermediates and/or structures of Formula (I), in which case they are reported as multiplets, partially overlapping with said solvent or impurity. In some cases, the structures of the end-products of the Formula (I) might appear as rotamers in the NMR-spectrum, in which instances only peaks of the major rotamer are reported. In some cases, the structures of the end-products of Formula (I) might appear as rotamers in more equal portions, in such instances the peaks of such rotamers are either reported as multiplets, if the signals of said rotamers are partially overlapping, or as individual peaks, if the signals of said rotamers are well separated. Electrospray mass spectral data were obtained using a Waters Acquity UPLC coupled to a Waters single quadrupole mass spectrometer or similar equipment, acquiring both positive and negative ion data, and generally, only ions relating to the parent structure are reported; high resolution electrospray mass spectral data were obtained using a Waters XEVO qToF mass spectrometer or similar equipment, coupled to a Waters Acquity UPLC, acquiring either positive and negative ion data, and generally, only ions relating to the parent structure are reported
(xiv) intermediates were not necessarily fully purified but their structures and purity were assessed by TLC, analytical HPLC/UPLC, and/or NMR analysis and/or mass spectrometry;
(xv) unless stated otherwise compounds containing an asymmetric carbon and/or sulfur atom were not resolved;
(xvi) in general Examples and Intermediate compounds are named using ChemDraw Professional version 20.0.2.51 or version 21.0.0 from PerkinElmer. ChemDraw Professional version 20.0.2.51 or version 21.0.0 generates the names of chemical structures using the Cahn-Ingold-Prelog (CIP) rules for stereochemistry and follows IUPAC rules as closely as possible when generating chemical names. Stereoisomers are differentiated from each other by stereodescriptors cited in names and assigned in accordance with the CIP rules.

ChemDraw is optionally using labels in the graphical representation of stereocenters such as ‘&’ and ‘or’ to describe the configuration of the stereochemical centers present in the structure.

In general chemical structures of Examples and Intermediates containing the label ‘&’ at a stereocenter, means the configuration of such Example or Intermediate at that stereocenter is a mixture of both (R) and (S); and a label ‘or’ means the configuration of such Example or Intermediate at that stereocenter is either (S) or (R). Absolute, unspecified, ‘&’, and ‘or’ stereocenters can all be present in a single structure.

In general for structures of Examples and Intermediates where all of the stereocenters are designated as ‘&’, the structure is named with a “rac-” prefix. For structures of Examples and Intermediates where all of the stereocenters are designated as ‘or’, the structure is named with a “rel-” prefix.

In general Examples and Intermediate compounds are named using the descriptors (RS) and (SR) to denote general ‘&’ centers for chemical structures with multiple chiral centers where only some are designated as ‘&’. The descriptors (R*) and (S*) are used to denote the general ‘or’ centers for chemical structures with multiple chiral centers where only some are designated as ‘or’.

In general, for structures of Examples and Intermediates where all stereocenters present are racemic, no flag is designated to the stereocenter(s) and the structure is drawn with a straight bond at each stereocenter.

In general, for structures of Examples and Intermediates where two or more stereocenters are present in a ring and fixed to each other and do not vary independently of each other, e.g. are cis or trans to each other, said stereocenters are drawn with stereobonds representing their internal relationship. Said stereocenters are labelled with an “&1” flag representing a mixture of cis-configuration or a mixture of trans-configuration, or an “or1” flag representing a single cis-isomer or a single trans-isomer with unknown absolute stereochemistry. In general, should the structure of said Example or Intermediate further contain one or more stereocenters that are racemic and not fixed in relation to the former stereocenters, said stereocenter(s) is drawn with a straight bond at said stereocenters.

In general the descriptors (r) and (s) are used to describe the absolute configuration of any pseudoasymmetric centers in the structures of Examples and Intermediates.

In general the label “Isomer 1” corresponds to the first eluted isomer, and “Isomer 2” corresponds to the second eluted isomer, on a given chiral HPLC column and eluent, and are used to distinguish two isomers containing one or more stereocenters with absolute unknown configuration;
(xvii) All crystallographic measurements were performed at 175K on a Bruker Smart Apex II diffractometer operating in the ω scans mode. The intensity data were collected within the θₘₐₓ≤26.0° using Mo-K_{α} radiation (λ=0.71078 Å). The structure was solved by direct methods and refined by the full-matrix least-squares technique in the anisotropic approximation for non-hydrogen atoms using the Bruker SHELXTL program package.
(xviii) in addition to the ones mentioned above, the following abbreviations and units have been used:

### Abbreviations

AcOH acetic acid
aq aqueous
BINAP rac (2,2′-bis(diphenylphosphaneyl)-1,1′-binaphthalene CAS 98327-87-8
Boc tert-butyloxycarbonyl
t-Bu tert-butyl
calcd calculated
DCM dichloromethane
DEA diethyl amine
DIPEA N,N-diisopropylethylamine
DMF N,N-dimethylformamide
DMSO dimethyl sulfoxide
EC50 half maximal effective concentration
ESI electrospray ionization
Et₃N triethylamine
EtOAc ethyl acetate
EtOH ethanol
FA formic acid
(g) gas
HPLC high performance liquid chromatography
HRMS high resolution mass spectrometry
IC50 half maximal inhibitory concentration
ID inner diameter
IPA propan-2-ol
(l) liquid
LCMS liquid chromatography mass spectrometry
MeCN acetonitrile
MeOH methanol
MS mass spectrometry
MTBE methyl tert-butyl ether
NMR nuclear magnetic resonance
Pd₂(dba)₃ Tris(dibenzylideneacetone)dipalladium(O)
Pd(dppf)Cl₂ [1,1′-Bis(diphenylphosphino)ferrocene]dichloropalladium(II)
pTsOH para-toluenesulfonic acid
rt room temperature
RuPhos dicyclohexyl(2′,6′-diisopropoxy-[1,1′-biphenyl]-2-yl)phosphane
sat saturated
SFC supercritical fluid chromatography
TEA triethylamine
TFA trifluoroacetic acid
THE tetrahydrofuran
TLC thin layer chromatography
UPLC ultra performance liquid chromatography
UV ultraviolet
VCD vibrational circular dichroism
Xantphos (9,9-dimethyl-9H-xanthene-4,5-diyl)bis(diphenylphosphane)

### Units

atm atmosphere
C Celcius
g gram
h hour(s)
L liter
M mole per liter
mg milligram
MHz megahertz
min minute(s)
mL milliliter
mm millimeter
mM millimole per liter
mmol millimole(s)
μCi microcurie
μm micrometer
μL microliter
nL nanoliter
nm nanometer
pM picomol per liter
is ppm parts per million
w/v weight by volume

### Intermediates

### Intermediate 1

### Methyl (S)-3-methoxy-4-nitro-5-((oxetan-2-ylmethyl)amino)benzoate

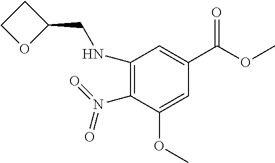 [SMILES: COC(=O)c1cc(NC[C@@H]2CCO2)c([N+](=O)[O-])c(OC)c1]

K₂CO₃ (5.43 g, 39.27 mmol) was added to a solution of methyl 3-fluoro-5-methoxy-4-nitrobenzoate (3 g, 13.09 mmol) and (S)-oxetan-2-ylmethanamine (1.14 g, 13.09 mmol) in THF/DMF (5:2, 110 mL) and the reaction mixture was stirred at 90° C. for 16 h. The solvent was removed under reduced pressure, and the residue was suspended in water (250 mL). The aqueous layer was extracted with EtOAc (3×250 mL), and the combined organic layer was dried over Na₂SO₄, filtered and evaporated at reduced pressure. The crude product was purified by straight phase flash column chromatography on silica (10-20% EtOAc in petroleum ether) to give the title compound (1.8 g, 46%) as a yellow solid; MS (ESI) m/z [M+H]⁺ 297.1.

### Intermediate 2

### Methyl (S)-4-amino-3-methoxy-5-((oxetan-2-ylmethyl)amino)benzoate

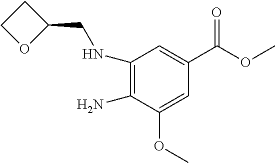 [SMILES: COC(=O)c1cc(NC[C@@H]2CCO2)c(N)c(OC)c1]

A suspension of Pd—C (0.144 g, 1.35 mmol) and methyl (S)-3-methoxy-4-nitro-5-((oxetan-2-ylmethyl)amino)benzoate Intermediate 1 (4.0 g, 13.50 mmol) in THF (100 mL) was stirred under an atmosphere of H₂(g) at 2 atm and 15° C. for 3 h. The reaction mixture was filtered through celite, and the filter cake was washed with MeOH (3×300 mL). The filtrate was concentrated under reduced pressure. The crude product was purified by straight phase flash column chromatography on silica (50-70% EtOAc in petroleum ether) to give the title compound (3.0 g, 83%) as a light yellow solid; MS (ESI) m/z [M+H]⁺ 267.3.

### Intermediate 3

### Methyl (S)-2-(chloromethyl)-4-methoxy-1-(oxetan-2-ylmethyl)-1H-benzo[d]imidazole-6-carboxylate

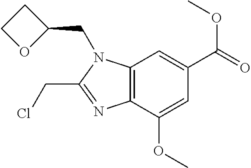 [SMILES: COC(=O)c1cc(OC)c2nc(CCl)n(C[C@@H]3CCO3)c2c1]

pTsOH (0.119 g, 0.63 mmol) was added to a solution of methyl (S)-4-amino-3-methoxy-5-((oxetan-2-ylmethyl)amino)benzoate Intermediate 2 (333 mg, 1.25 mmol) and 2-chloro-1,1,1-trimethoxyethane (387 mg, 2.50 mmol) in MeCN (10 mL) and the reaction mixture was stirred at 45° C. for 30 min. The reaction mixture was concentrated under reduced pressure and the residue was purified by straight phase flash column chromatography on silica (50-100% EtOAc in heptane) to give the title compound (155 mg, 38%); MS (ESI) m/z [M+H]⁺ 325.0.

### Intermediate 4

### (S)-5-Bromo-3-methoxy-2-nitro-N-((tetrahydrofuran-2-yl)methyl)aniline

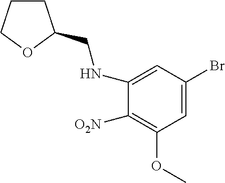 [SMILES: COc1cc(Br)cc(NC[C@@H]2CCCO2)c1[N+](=O)[O-]]

DIPEA (3.73 ml, 21.35 mmol) was added to a solution of (S)-(tetrahydrofuran-2-yl)methanamine (1.08 g, 10.68 mmol) and 5-bromo-1-fluoro-3-methoxy-2-nitrobenzene (2.67 g, 10.68 mmol) in MeCN (20 mL) and the reaction mixture was stirred at 57° C. for 3 h. The reaction mixture was concentrated at reduced pressure and the residue was purified by straight phase flash column chromatography on silica (0-20% EtOAc in heptane) to give the title product (2.39 g, 68%); MS (ESI) m/z [M+H]⁺ 333.1.

### Intermediate 5

### (S)-5-Bromo-3-methoxy-N1-((tetrahydrofuran-2-yl)methyl)benzene-1,2-diamine

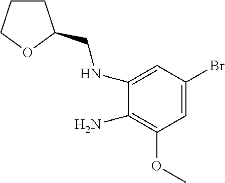 [SMILES: COc1cc(Br)cc(NC[C@@H]2CCCO2)c1N]

DIPEA (6.07 mL, 34.73 mmol) and HSiCl₃ (2.453 ml, 24.31 mmol) was added dropwise at 0° C. to a solution of (S)-5-bromo-3-methoxy-2-nitro-N-((tetrahydrofuran-2-yl)methyl)aniline Intermediate 4 (2.3 g, 6.95 mmol) in MeCN (20 mL). The reaction mixture was stirred at 0° C. for 2 min and then at rt for 30 min. NaHCO₃(aq, 10 mL) was added dropwise and the biphasic mixture was stirred at rt for 30 min and then extracted with EtOAc (2×5 mL). The combined organic layer was dried over MgSO₄, filtered and evaporated at reduced pressure to give the title compound (2.0 g, 96%); MS (ESI) m/z [M+H]⁺ 303.1.

### Intermediate 6

### Methyl (S)-4-amino-3-methoxy-5-(((tetrahydrofuran-2-yl)methyl)amino)benzoate

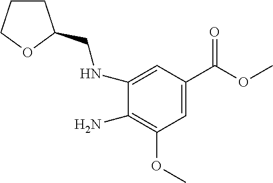 [SMILES: COC(=O)c1cc(NC[C@@H]2CCCO2)c(N)c(OC)c1]

DIPEA (8.70 mL, 49.80 mmol) was added to a suspension of (S)-5-bromo-3-methoxy-N1-((tetrahydrofuran-2-yl)methyl)benzene-1,2-diamine Intermediate 5 (1.5 g, 4.98 mmol) and Pd(dppf)Cl₂ (292 mg, 0.40 mmol) in MeOH (20 mL) and the reaction mixture was stirred under an atmosphere of CO(g) at 9 atm and 85° C. for 16 h. The reaction mixture was filtered through a pad of celite, and the filter cake was rinsed with MeOH (10 mL). The filtrate was concentrated at reduced pressure and the residue was purified by straight phase flash column chromatography on silica (20-60% EtOAc in heptane) to give the title compound (444 mg, 32%); MS (ESI) m/z [M+H]⁺ 281.2.

### Intermediate 7

### Methyl (S)-2-(chloromethyl)-4-methoxy-1-((tetrahydrofuran-2-yl)methyl)-1H-benzo[d]imidazole-6-carboxylate

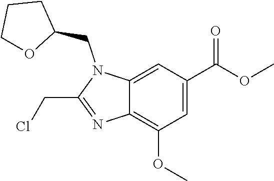 [SMILES: COC(=O)c1cc(OC)c2nc(CCl)n(C[C@@H]3CCCO3)c2c1]

pTSOH (149 mg, 0.78 mmol) was added to a solution of methyl (S)-4-amino-3-methoxy-5-(((tetrahydrofuran-2-yl)methyl)amino)benzoate Intermediate 6 (440 mg, 1.57 mmol) and 2-chloro-1,1,1-trimethoxyethane (485 mg, 3.14 mmol) in MeCN (10 mL) and the reaction mixture was stirred at 50° C. for 30 min. The reaction mixture was diluted with EtOAc (10 mL) and washed with NaHCO₃ (2×3 mL). The organic phase was dried over MgSO₄, filtered and evaporated at reduced pressure to give the title compound (450 mg, 85%); MS (ESI) m/z [M+H]⁺ 399.0.

### Intermediate 8

### (S)-5-Bromo-3-chloro-2-nitro-N-((tetrahydrofuran-2-yl)methyl)aniline

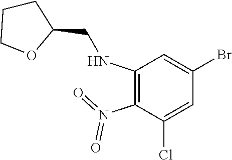 [SMILES: O=[N+]([O-])c1c(Cl)cc(Br)cc1NC[C@@H]1CCCO1]

DIPEA (3.45 mL, 19.77 mmol) was added to a solution of (S)-(tetrahydrofuran-2-yl)methanamine (1 g, 9.89 mmol) and 5-bromo-1-chloro-3-fluoro-2-nitrobenzene (2.52 g, 9.89 mmol) in MeCN (20 mL) and the reaction mixture was stirred at 45° C. for 2 h. The reaction mixture was evaporated at reduced pressure and the residue was purified by straight phase flash column chromatography on silica (0-20% EtOAc in heptane) to give the title compound (2.70 g, 81%); MS (ESI) m/z [M+H]⁺ 332.8.

### Intermediate 9

### (S)-5-Bromo-3-chloro-N1-((tetrahydrofuran-2-yl)methyl)benzene-1,2-diamine

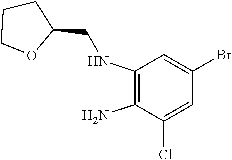 [SMILES: Nc1c(Cl)cc(Br)cc1NC[C@@H]1CCCO1]

Fe(s) (3.59 g, 64.37 mmol) was added to a mixture of (S)-5-bromo-3-chloro-2-nitro-N-((tetrahydrofuran-2-yl)methyl)aniline Intermediate 8 (2.7 g, 8.05 mmol) and NH₄Cl (3.44 g, 64.37 mmol) in water (5 mL) and MeOH (15 mL), and the reaction mixture was heated at 60° C. for 1 h. The reaction mixture was filtered through a pad of Celite. The precipitate formed in the filtrate was filtered off, rinsed with water, and dried in vacuo, to give the title compound (2.20 g, 89%); MS (ESI) m/z [M+H]⁺ 305.11.

### Intermediate 10

### Methyl (S)-4-amino-3-chloro-5-(((tetrahydrofuran-2-yl)methyl)amino)benzoate

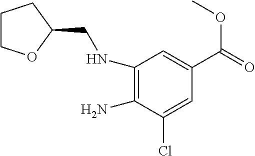 [SMILES: COC(=O)c1cc(Cl)c(N)c(NC[C@@H]2CCCO2)c1]

DIPEA (12.57 mL, 71.99 mmol) was added to a mixture of (S)-5-bromo-3-chloro-N1-((tetrahydrofuran-2-yl)methyl)benzene-1,2-diamine Intermediate 9 (2.2 g, 7.20 mmol), Pd(dppf)Cl₂ (421 mg, 0.58 mmol) in MeOH (20 mL) and the reaction mixture was stirred under an atmosphere of CO(g) at 5 atm and 80° C. for 16 h, and then at 9 bar and 80° C. for 72 h. The reaction mixture was filtered through a pad of Celite, and the pad was rinsed with MeOH (10 mL), and the filtrate was concentrated at reduced pressure. The residue was purified by straight phase flash column chromatography on silica (20-60% EtOAc in heptane) to give the title compound (960 mg, 47%); MS (ESI) m/z [M+H]⁺ 285.0.

### Intermediate 11

### Methyl (S)-4-chloro-2-(chloromethyl)-1-((tetrahydrofuran-2-yl)methyl)-1H-benzo[d]imidazole-6-carboxylate

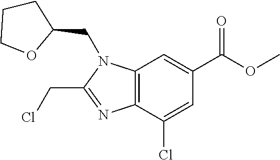 [SMILES: COC(=O)c1cc(Cl)c2nc(CCl)n(C[C@@H]3CCCO3)c2c1]

pTsOH (160 mg, 0.84 mmol) was added to a solution of methyl (S)-4-amino-3-chloro-5-(((tetrahydrofuran-2-yl)methyl)amino)benzoate Intermediate 10 (480 mg, 1.69 mmol) and 2-chloro-1,1,1-trimethoxyethane (521 mg, 3.37 mmol) in MeCN (10 mL) and the reaction mixture was stirred at 45° C. for 90 min. The reaction mixture was concentrated at reduced pressure and the residue was purified by straight phase flash column chromatography on silica (50-100% EtOAc in heptane) to give the title compound (280 mg, 48%); MS (ESI) m/z [M+H]⁺ 343.0.

### Intermediate 12

### Methyl 3-((2-(1H-pyrazol-1-yl)ethyl)amino)-4-nitrobenzoate

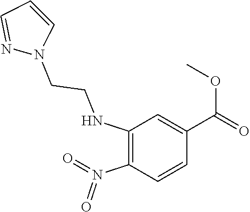 [SMILES: COC(=O)c1ccc([N+](=O)[O-])c(NCCn2cccn2)c1]

A mixture of methyl 3-fluoro-4-nitrobenzoate (2.34 g, 11.75 mmol), 2-(1H-pyrazol-1-yl)ethan-1-amine (1.30 g, 11.70 mmol) and DIPEA (6 mL, 34.35 mmol) in MeCN (20 mL) was stirred at 45° C. overnight. The reaction mixture was cooled and concentrated, and the residue was purified by flash chromatography on silica (0-50% EtOAC in heptane). Product containing fractions were pooled and concentrated to give the title compound (1.82 g, 54%) as an orange powder; MS (ESI) m/z [M+H]⁺ 281.17.

### Intermediate 13

### Methyl 3-((2-(1H-pyrazol-1-yl)ethyl)amino)-4-aminobenzoate

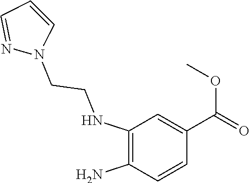 [SMILES: COC(=O)c1ccc(N)c(NCCn2cccn2)c1]

A mixture of methyl 3-((2-(1H-pyrazol-1-yl)ethyl)amino)-4-nitrobenzoate Intermediate 12 (1.82 g, 6.27 mmol) and Pd—C (0.133 g, 0.06 mmol) in AcOH (50 mL) was stirred under an atmosphere of H₂ (g) (1 bar) at rt for 2 h. The reaction mixture was filtered through a syringe filter, and the filtrate was concentrated. The residue was dissolved in EtOAc (150 mL) and washed with sat NaHCO₃ (aq, 50 mL) and water (50 mL). The organic phase was dried over MgSO₄, filtered, and concentrated to give the title compound (1.59 g, 97%) as a yellow oil which solidified upon standing; MS (ESI) m/z [M+H]⁺ 261.18.

### Intermediate 14

### Methyl (S)-4-nitro-3-(((tetrahydrofuran-2-yl)methyl)amino)benzoate

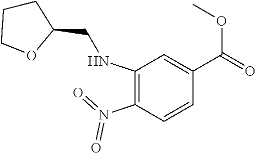 [SMILES: COC(=O)c1ccc([N+](=O)[O-])c(NC[C@@H]2CCCO2)c1]

DIPEA (6.91 mL, 39.55 mmol) was added to a solution of (S)-(tetrahydrofuran-2-yl)methanamine (2.0 g, 19.8 mmol) and methyl 3-fluoro-4-nitrobenzoate (3.94 g, 19.8 mmol) in MeCN (20 mL), and the reaction mixture was stirred at 35° C. for 18 h. The reaction mixture was evaporated and the residue was purified by flash chromatography on silica (0-40% EtOAc in heptane) to give the title compound (4.34 g, 78%); 1H NMR (500 MHz, CDCl3) 1.65-1.76 (1H, m), 1.94-2.03 (2H, m), 2.11 (1H, td), 3.35-3.43 (1H, m), 3.52 (1H, d), 3.79-3.86 (1H, m), 3.94 (4H, s), 4.22 (1H, qd), 7.24 (1H, dd), 7.59 (1H, d), 8.22 (2H, d).

### Intermediate 15

### Methyl (S)-4-amino-3-(((tetrahydrofuran-2-yl)methyl)amino)benzoate

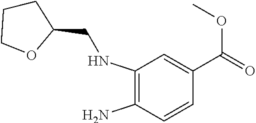 [SMILES: COC(=O)c1ccc(N)c(NC[C@@H]2CCCO2)c1]

Pd—C (5%, 0.330 g, 3.10 mmol) was added to a solution of methyl (S)-4-nitro-3-(((tetrahydrofuran-2-yl)methyl)amino)benzoate Intermediate 14 (4.34 g, 15.48 mmol) in MeOH (40 mL) and the reaction mixture was stirred under an atmosphere of H₂(g) (1 atm) at rt for 30 min. The catalyst was filtered off and the filtrate was concentrated under reduced pressure to give the title compound (3.70 g, 9%); MS (ESI) m/z [M+H]⁺ 251.25.

### Intermediate 16

### Methyl (S)-2-(chloromethyl)-1-((tetrahydrofuran-2-yl)methyl)-1H-benzo[d]imidazole-6-carboxylate

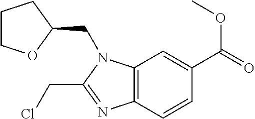 [SMILES: COC(=O)c1ccc2nc(CCl)n(C[C@@H]3CCCO3)c2c1]

pTsOH (0.281 g, 1.48 mmol) was added to a solution of methyl (S)-4-amino-3-(((tetrahydrofuran-2-yl)methyl)amino)benzoate Intermediate 15 (3.7 g, 14.78 mmol) and 2-chloro-1,1,1-trimethoxyethane (2.74 g, 17.74 mmol) in MeCN (10 mL), and the reaction mixture was stirred at 45° C. for 90 min. The solvent was evaporated and the residue was purified by flash chromatography on silica (50-100% EtOAc in heptane) to give the title compound (4.30 g, 94%); MS (ESI) m/z [M+H]⁺ 309.0.

### Intermediate 17

### Methyl (S)-3-fluoro-4-nitro-5-((oxetan-2-ylmethyl)amino)benzoate

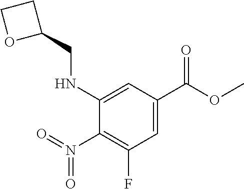 [SMILES: COC(=O)c1cc(F)c([N+](=O)[O-])c(NC[C@@H]2CCO2)c1]

DIPEA (8.45 mL, 48.36 mmol) was added to a solution of methyl 3,5-difluoro-4-nitrobenzoate (3.50 g, 16.12 mmol) and (S)-oxetan-2-ylmethanamine (1.40 g, 16.12 mmol) in THF/DMF (125 mL, 5:2), and the reaction mixture was stirred at 20° C. for 4 h. The solvent was removed under reduced pressure, and the residue was suspended in water (300 mL). The aqueous layer was extracted with EtOAc (3×500 mL), and the combined organic layer was dried over Na₂SO₄, filtered and evaporated. The crude product was purified by straight phase flash chromatography on silica (gradient: 10-20% EtOAc in petroleum ether) to give the title compound (4.50 g, 98%) as a yellow solid; MS (ESI) m/z [M+H]⁺ 285.0.

### Intermediate 18

### Methyl (S)-4-amino-3-fluoro-5-((oxetan-2-ylmethyl)amino)benzoate

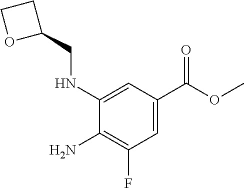 [SMILES: COC(=O)c1cc(F)c(N)c(NC[C@@H]2CCO2)c1]

A suspension of methyl (S)-3-fluoro-4-nitro-5-((oxetan-2-ylmethyl)amino)benzoate Intermediate 17 (4.2 g, 14.78 mmol) and 10% Pd—C (1.57 g, 1.48 mmol) in THF (150 mL) was stirred under an atmosphere of H₂ (g) at 3 atm and 25° C. for 2 h. The reaction mixture was filtered through celite and the filter cake was washed with MeOH (3×100 mL). The filtrate was concentrated under reduced pressure. The crude product was purified by straight phase flash chromatography on silica (gradient: 70-80% EtOAc in petroleum ether), to give the title compound (3.20 g, 85%) as a light red solid; MS (ESI) m/z [M+H]⁺ 254.95.

### Intermediate 19

### Methyl (S)-2-(chloromethyl)-4-fluoro-1-(oxetan-2-ylmethyl)-1H-benzo[d]imidazole-6-carboxylate

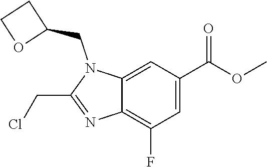 [SMILES: COC(=O)c1cc(F)c2nc(CCl)n(C[C@@H]3CCO3)c2c1]

pTsOH (0.108 g, 0.57 mmol) was added to a solution of methyl (S)-4-amino-3-fluoro-5-((oxetan-2-ylmethyl)amino)benzoate Intermediate 18 (1.45 g, 5.70 mmol) and 2-chloro-1,1,1-trimethoxyethane (1.06 g, 6.84 mmol) in MeCN (10 mL) and the reaction mixture was stirred at rt for 18 h. The solvent was evaporated at reduced pressure and the crude compound was purified by straight phase flash chromatography on silica (gradient: 50-100% EtOAc in heptane) to give the title compound (1.54 g, 86%); MS (ESI) m/z [M+H]⁺ 313.26.

### Intermediate 20

### rac-tert-Butyl (4aR,7aS)-4-(2-(5-chloropyridin-2-yl)-2-methylbenzo[d][1,3]dioxol-4-yl)hexahydrofuro[3,4-b]pyrazine-1(2H)-carboxylate

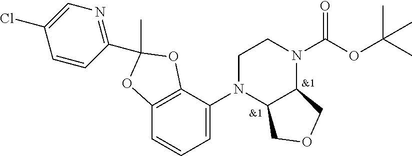 [SMILES: CC(C)(C)OC(=O)N1CCN(c2cccc3c2OC(C)(c2ccc(Cl)cn2)O3)[C@@H]2COC[C@@H]21]

Pd₂(dba)₃ (454 mg, 0.50 mmol) was added to as suspension of 2-(4-bromo-2-methylbenzo[d][1,3]dioxol-2-yl)-5-chloropyridine (WO2020234726) (1.62 g, 4.96 mmol), rac-tert-butyl (4aR,7aS)-hexahydrofuro[3,4-b]pyrazine-1(2H)-carboxylate (1.36 g, 5.95 mmol), RuPhos (926 mg, 1.98 mmol) and sodium 2-methylpropan-2-olate (1.91 g, 19.84 mmol) in degassed toluene (10 mL). The reaction mixture was evacuated and backfilled with N₂(g) (×3) and then stirred at 35° C. for 18 h. The reaction mixture was cooled to rt and filtered through a pad of celite and the filter cake was rinsed with toluene. The filtrate was concentrated at reduced pressure and the residue was purified by straight phase flash column chromatography on silica (0-20% EtOAc in heptane) to give the title product (1.38 g, 59%); MS (ESI) m/z [M+H]⁺ 474.4.

### Intermediate 21

### rac-tert-Butyl (4aR,7aS)-4-(2-(4-chloro-2-fluorophenyl)-2-methylbenzo[d][1,3]dioxol-4-yl)hexahydrofuro[3,4-b]pyrazine-1(2H)-carboxylate

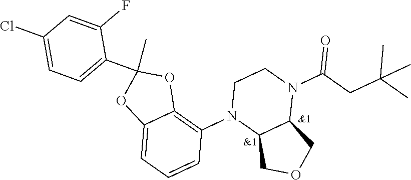 [SMILES: CC(C)(C)CC(=O)N1CCN(c2cccc3c2OC(C)(c2ccc(Cl)cc2F)O3)[C@@H]2COC[C@@H]21]

Pd₂(dba)₃ (0.800 g, 0.87 mmol) was added to a suspension of 4-bromo-2-(4-chloro-2-fluorophenyl)-2-methylbenzo[d][1,3]dioxole (WO2020234726) (3 g, 8.73 mmol), rac-tert-butyl (4aR,7aS)-hexahydrofuro[3,4-b]pyrazine-1(2H)-carboxylate (1.9 g, 8.32 mmol), RuPhos (0.815 g, 1.75 mmol) and sodium 2-methylpropan-2-olate (3.36 g, 34.93 mmol) in degassed toluene (10 mL). The reaction mixture was evacuated and backfilled with N₂(g) (×3) and then stirred at 37° C. for 16 h. The reaction mixture was cooled to rt and filtered through a pad of Celite. The pad was rinsed with MeOH (10 mL) and the filtrate was evaporated. The residue was purified by straight phase flash column chromatography on silica (0-20% EtOAc in heptane) to give the title compound (4.00 g, 93%); MS (ESI) m/z [M+H]⁺ 491.3.

### Intermediate 22

### rac-(4aR,7aS)-1-(2-(4-Chloro-2-fluorophenyl)-2-methylbenzo[d][1,3]dioxol-4-yl)octahydrofuro[3,4-b]pyrazine

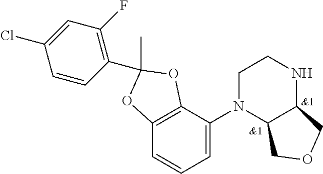 [SMILES: CC1(c2ccc(Cl)cc2F)Oc2cccc(N3CCN[C@H]4COC[C@H]43)c2O1]

A solution of HCl in 1,4-dioxane (4 M, 1.5 mL, 6.00 mmol) was added to a solution of rac-tert-butyl (4aR,7aS)-4-(2-(4-chloro-2-fluorophenyl)-2-methylbenzo[d][1,3]dioxol-4-yl)hexahydrofuro[3,4-b]pyrazine-1(2H)-carboxylate Intermediate 21 (441 mg, 0.90 mmol) in 1,4-dioxane (5 mL), and the reaction mixture was stirred at rt overnight. The reaction mixture was concentrated under reduced pressure to give the title compound as an HCl salt (0.417 g, 100%); MS (ESI) m/z [M+H]⁺ 391.33.

### Intermediate 23

### Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate

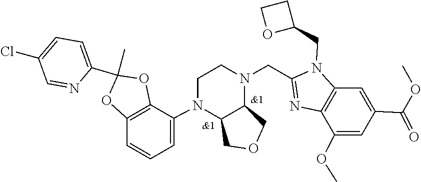 [SMILES: COC(=O)c1cc(OC)c2nc(CN3CCN(c4cccc5c4OC(C)(c4ccc(Cl)cn4)O5)[C@@H]4COC[C@@H]43)n(C[C@@H]3CCO3)c2c1]

### Step a) rac-(4aR,7aS)-1-(2-(5-Chloropyridin-2-yl)-2-methylbenzo[d][1,3]dioxol-4-yl)octahydrofuro[3,4-b]pyrazine

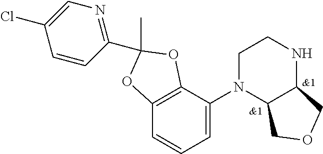 [SMILES: CC1(c2ccc(Cl)cn2)Oc2cccc(N3CCN[C@H]4COC[C@H]43)c2O1]

TFA (187 μL, 2.43 mmol) was added to a solution of rac-tert-butyl (4aR,7aS)-4-(2-(5-chloropyridin-2-yl)-2-methylbenzo[d][1,3]dioxol-4-yl)hexahydrofuro[3,4-b]pyrazine-1(2H)-carboxylate Intermediate 20 (230 mg, 0.49 mmol) in DCM (3.0 mL) and the reaction mixture was stirred at rt for 20 min. The reaction mixture was evaporated at reduced pressure to give the TFA-salt of the sub-title compound; MS (ESI) m/z [M+H]⁺ 374.2.

### Step b) Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate

The crude product from Step a) was dissolved in MeCN (3 mL) and K₂CO₃ (201 mg, 1.46 mmol) and methyl (S)-2-(chloromethyl)-4-methoxy-1-(oxetan-2-ylmethyl)-1H-benzo[d]imidazole-6-carboxylate Intermediate 3 (155 mg, 0.48 mmol) were added, and the reaction mixture was heated at 75° C. for 2 h, and then at 85° C. for 1 h. The reaction mixture was cooled to rt and filtered through a pad of Celite. The filtrate was evaporated under reduced pressure and the residue was purified by straight phase flash column chromatography on silica (30-100% EtOAc in heptane) to give the title compound (170 mg, 53%); MS (ESI) m/z [M+H]⁺ 662.5.

### Intermediate 24

### Methyl 2-((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 1

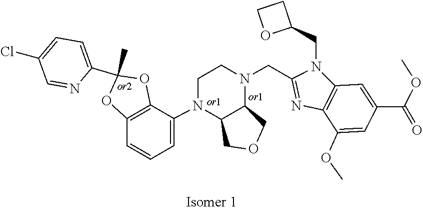 [SMILES: COC(=O)c1cc(OC)c2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 25

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 3

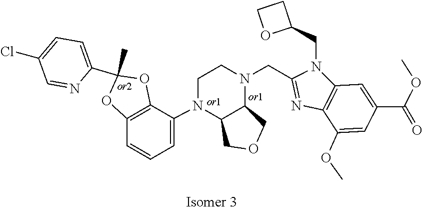 [SMILES: COC(=O)c1cc(OC)c2nc(CN3CCN(c4cccc5c4O[C@@](C)(c4ccc(Cl)cn4)O5)[C@@H]4COC[C@@H]43)n(C[C@@H]3CCO3)c2c1]

The diastereomers of methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate Intermediate 23 (170 mg, 0.26 mmol) were separated by chiral chromatography on a LUX C3 (OJ) column (250×20 mm, 5 μm), eluted with 12% MeOH/DEA (100/20 mM) in CO₂, 125 bar, at a flow rate of 70 mL/min and detected at 220 nm;
the first eluted compound mixture was collected and evaporated to yield a mixture of isomers (52 mg) and;
is the second eluted compound was collected and evaporated to yield the title compound Isomer 3, Intermediate 25 (35 mg); MS (ESI) m/z [M+H]⁺ 662.3.

The stereoisomers of the first eluted compound mixture (52 mg) were separated by chiral chromatography on a YMC SA (IA) column (250×30 mm, 5 μm), eluted with 25% MeOH/DEA (100/20 mM) in CO₂, 130 bar, at a flow rate of 120 mL/min and detected at 220 nm;
the first eluted compound was collected and evaporated to yield the title compound Isomer 1, Intermediate 24 (23 mg); MS (ESI) m/z [M+H]⁺ 662.4.

### Intermediate 26

### Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylate

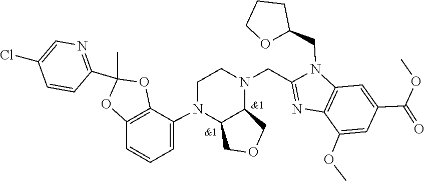 [SMILES: COC(=O)c1cc(OC)c2nc(CN3CCN(c4cccc5c4OC(C)(c4ccc(Cl)cn4)O5)[C@@H]4COC[C@@H]43)n(C[C@@H]3CCCO3)c2c1]

### Step a) rac-(4aR,7aS)-1-(2-(5-Chloropyridin-2-yl)-2-methylbenzo[d][1,3]dioxol-4-yl)octahydrofuro[3,4-b]pyrazine

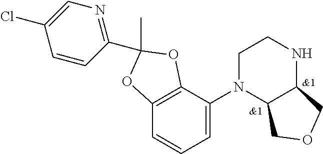 [SMILES: CC1(c2ccc(Cl)cn2)Oc2cccc(N3CCN[C@H]4COC[C@H]43)c2O1]

TFA (171 μL, 2.22 mmol) was added to a solution of rac-tert-butyl (4aR,7aS)-4-(2-(5-chloropyridin-2-yl)-2-methylbenzo[d][1,3]dioxol-4-yl)hexahydrofuro[3,4-b]pyrazine-1(2H)-carboxylate Intermediate 20 (210 mg, 0.44 mmol) in DCM (3.0 mL), and the reaction mixture was stirred at rt for 30 min. The reaction mixture was evaporated at reduced pressure and co-evaporate with toluene (10 mL) to give the TFA salt of the sub-title compound; MS (ESI) m/z [M+H]⁺ 374.0.

### Step b) Methyl 2-(((4aRS,7aSR)-4-(2-(5-chloropyridin-2-yl)-2-methylbenzo[d][1,3]dioxol-4-yl)hexahydrofuro[3,4-b]pyran-1(2H)-yl)methyl)-4-methoxy-1-(((S)tetrahydrofuran-2-yl)methyl)-1H-benzo[d]imidazole-6-carboxylate

The crude product from Step a) was dissolved in MeCN (3 mL), and K₂CO₃ (184 mg, 1.33 mmol) and methyl (S)-2-(chloromethyl)-4-methoxy-1-((tetrahydrofuran-2-yl)methyl)-1H-benzo[d]imidazole-6-carboxylate Intermediate 7 (150 mg, 0.44 mmol) were added, and the reaction mixture was heated at 60° C. for 18 h. The reaction mixture was cooled to rt and filtered through a pad of Celite. The filtrate was evaporated under reduced pressure and the residue was purified by straight phase flash column chromatography on silica (30-100% EtOAc in heptane, then 4% EtOH in EtOAc) to give the title compound (200 mg, 67%); MS (ESI) m/z [M+H]⁺ 676.4.

### Intermediate 27

### Methyl 2-((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylate, Isomer 3

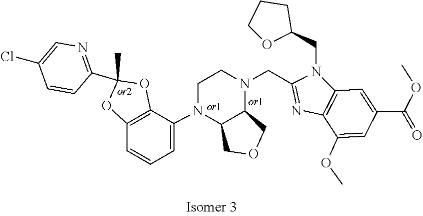 [SMILES: COC(=O)c1cc(OC)c2nc(CN3CCN(c4cccc5c4O[C@@](C)(c4ccc(Cl)cn4)O5)[C@@H]4COC[C@@H]43)n(C[C@@H]3CCCO3)c2c1]

### Intermediate 28

### Methyl 2-((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylate, Isomer 4

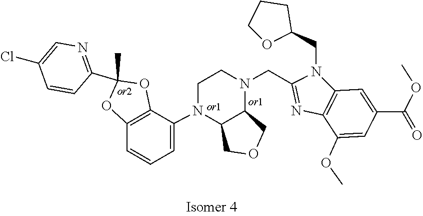 [SMILES: COC(=O)c1cc(OC)c2nc(CN3CCN(c4cccc5c4O[C@@](C)(c4ccc(Cl)cn4)O5)[C@@H]4COC[C@@H]43)n(C[C@@H]3CCCO3)c2c1]

The diastereomers of methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylate Intermediate 26 (200 mg, 0.30 mmol) were separated by chiral chromatography on a YMC SJ (imob OJ) column (250×30 mm, 5 μm), eluted with 15% EtOH/DEA (100/20 mM) in CO₂, 130 bar, at a flow rate of 130 mL/min and detected at 220 nm;
the second eluted compound was collected and evaporated to yield the title compound Isomer 3, Intermediate 27 (36 mg); MS (ESI) m/z [M+H]⁺ 676.4.
the third eluted compound was collected and evaporated to yield the title compound Isomer 4, Intermediate 28 (22 mg); MS (ESI) m/z [M+H]⁺ 676.4.

### Intermediate 29

### Methyl 2-(((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate

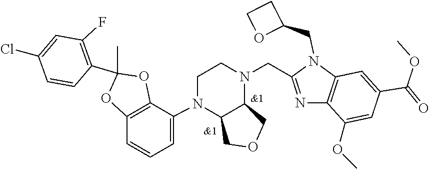 [SMILES: COC(=O)c1cc(OC)c2nc(CN3CCN(c4cccc5c4OC(C)(c4ccc(Cl)cc4F)O5)[C@@H]4COC[C@@H]43)n(C[C@@H]3CCO3)c2c1]

### Step a) rac-(4aR,7aS)-1-(2-(4-Chloro-2-fluorophenyl)-2-methylbenzo[d][1,3]dioxol-4-yl)octahydrofuro[3,4-b]pyrazine

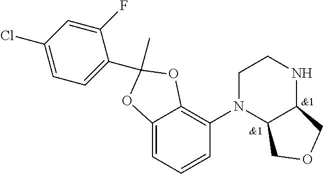 [SMILES: CC1(c2ccc(Cl)cc2F)Oc2cccc(N3CCN[C@H]4COC[C@H]43)c2O1]

TFA (392 μL, 5.09 mmol) was added to a solution of rac-tert-butyl (4aR,7aS)-4-(2-(4-chloro-2-fluorophenyl)-2-methylbenzo[d][1,3]dioxol-4-yl)hexahydrofuro[3,4-b]pyrazine-1(2H)-carboxylate Intermediate 21 (500 mg, 1.02 mmol) in DCM (10 mL) and the reaction mixture was stirred at rt for 30 min. The reaction mixture was evaporated, and the residue was co-evaporated with toluene (10 mL) followed by MeCN (2×10 mL) to give the TFA salt of the subtitle compound; MS (ESI) m/z [M+H]⁺ 392.9.

### Step b) Methyl 2-((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate

The crude product from Step a) was dissolved in MeCN (10 mL) and K₂CO₃ (422 mg, 3.06 mmol) and methyl (S)-2-(chloromethyl)-4-methoxy-1-(oxetan-2-ylmethyl)-1H-benzo[d]imidazole-6-carboxylate Intermediate 3 (397 mg, 1.22 mmol) were added, and the reaction mixture was heated at 70° C. for 18 h. The reaction mixture was cooled to rt and EtOAc (10 mL) was added, and the mixture was washed with NaHCO₃ (aq, 2×5 mL). The organic layer was evaporated at reduced pressure and the residue was purified by straight phase flash column chromatography on silica (10-100% EtOAc in heptane). The product containing fractions were collected and evaporated and the crude compound was purified by preparative HPLC, PrepMethod C, (gradient: 30-95%) to give the title compound (265 mg, 38%); MS (ESI) m/z [M+H]⁺ 679.5.

### Intermediate 30

### Methyl 2-((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 1

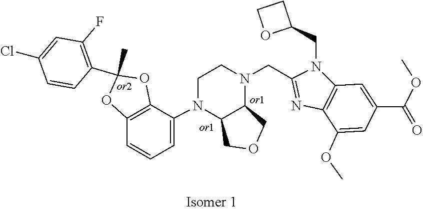 [SMILES: COC(=O)c1cc(OC)c2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 31

### Methyl 2-((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 2

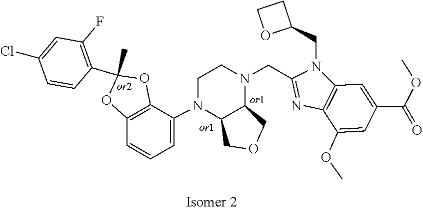 [SMILES: COC(=O)c1cc(OC)c2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 32

### Methyl 2-((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 4

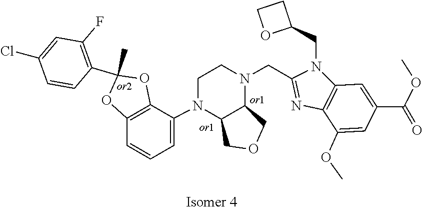 [SMILES: COC(=O)c1cc(OC)c2nc(CN3CCN(c4cccc5c4O[C@@](C)(c4ccc(Cl)cc4F)O5)[C@@H]4COC[C@@H]43)n(C[C@@H]3CCO3)c2c1]

The diastereoisomers of methyl 2-(((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-4-methoxy-1—(((S)-oxetan-2-yl)methyl)-1H-benzo[d]imidazole-6-carboxylate Intermediate 29 (265 mg) were separated by chiral chromatography on a YMC SA (IA) column (250×30 mm, 5 μm), eluted with 30% EtOH/DEA (100/20 mM) in CO₂, 130 bar, at a flow rate of 120 mL/min and detected at 220 nm;
the first eluted compound mixture was collected and evaporated to yield a mixture of isomers (116 mg) and;
the second eluted compound mixture was collected and evaporated to yield a mixture of isomers (123 mg).

The stereoisomers of the first eluted compound mixture (116 mg) were separated by chiral chromatography on a LUX C3 (OJ) column (250×30 mm, 5 μm), eluted with 17-22% EtOH/DEA (100/20 mM) in CO₂, 120 bar, at a flow rate of 100 mL/min and detected at 220 nm;
the first eluted compound was collected and evaporated to yield the title compound Isomer 1, Intermediate 30 (58 mg); MS (ESI) m/z [M+H]⁺ 679.4;
is the second eluted compound was collected and evaporated to yield the title compound Isomer 2, Intermediate 31 (48 mg); MS (ESI) m/z [M+H]⁺ 679.5.

The stereoisomers of the second eluted compound mixture (123 mg) were separated by chiral chromatography on a LUX C3 (OJ) column (250×30 mm, 5 μm), eluted with 12-17% EtOH/DEA (100/20 mM) in CO₂, 120 bar, at a flow rate of 100 mL/min and detected at 220 nm; the second eluted compound was collected and evaporated to yield the title compound Isomer 4, Intermediate 32 (40 mg); MS (ESI) m/z [M+H]⁺ 679.57.

### Intermediate 33

### Methyl 4-chloro-2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate

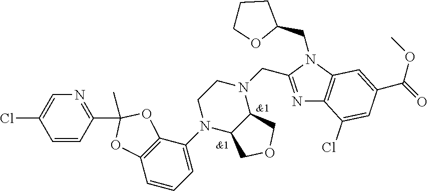 [SMILES: COC(=O)c1cc(Cl)c2nc(CN3CCN(c4cccc5c4OC(C)(c4ccc(Cl)cn4)O5)[C@@H]4COC[C@@H]43)n(C[C@@H]3CCCO3)c2c1]

### Step a) rac-(4aR,7aS)-1-(2-(5-Chloropyridin-2-yl)-2-methylbenzo[d][1,3]dioxol-4-yl)octahydrofuro[3,4-b]pyrazine

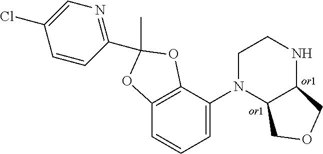 [SMILES: CC1(c2ccc(Cl)cn2)Oc2cccc(N3CCN[C@H]4COC[C@H]43)c2O1]

TFA (293 μL, 3.80 mmol) was added to a solution of rac-tert-butyl (4aR,7aS)-4-(2-(5-chloropyridin-2-yl)-2-methylbenzo[d][1,3]dioxol-4-yl)hexahydrofuro[3,4-b]pyrazine-1(2H)-carboxylate Intermediate 20 (360 mg, 0.76 mmol) in DCM (3 mL) and the reaction mixture was stirred at rt for 1 h. The reaction mixture was evaporated, and the residue was co-evaporated with toluene (10 mL) followed by MeCN (2×10 mL); MS (ESI) m/z [M+H]⁺ 374.3.

### Step b) Methyl 4-chloro-2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate

The crude product from Step a) was dissolved in MeCN (10 mL) and K₂CO₃ (315 mg, 2.28 mmol) and methyl (S)-4-chloro-2-(chloromethyl)-1-((tetrahydrofuran-2-yl)methyl)-1H-benzo[d]imidazole-6-carboxylate Intermediate 11 (280 mg, 0.82 mmol) were added, and the reaction mixture was heated at 60° C. for 18 h. The reaction mixture was cooled to rt and the reaction mixture was filtered through Celite. The filtrate was evaporated at reduced pressure and the residue was purified by straight phase flash column chromatography on silica (30-100% EtOAc in heptane, then 4% EtOH in EtOAc). The product containing fractions were collected and evaporated and the crude compound was purified by preparative HPLC, PrepMethod C, (gradient: 50-95%) to give the title compound (280 mg, 54%); MS (ESI) m/z [M+H]⁺ 680.4.

### Intermediate 34

### Methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate, Isomer 3

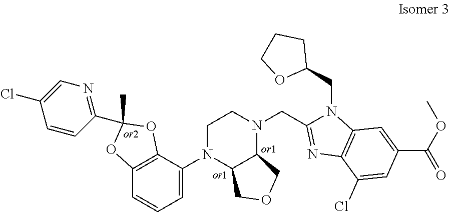 [SMILES: COC(=O)c1cc(Cl)c2nc(CN3CCN(c4cccc5c4O[C@@](C)(c4ccc(Cl)cn4)O5)[C@@H]4COC[C@@H]43)n(C[C@@H]3CCCO3)c2c1]

### Intermediate 35

### Methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate, Isomer 4

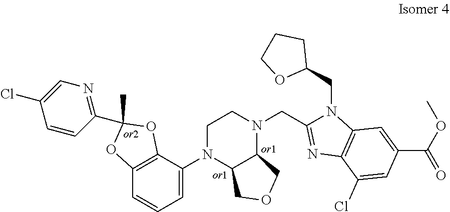 [SMILES: COC(=O)c1cc(Cl)c2nc(CN3CCN(c4cccc5c4O[C@@](C)(c4ccc(Cl)cn4)O5)[C@@H]4COC[C@@H]43)n(C[C@@H]3CCCO3)c2c1]

The diastereoisomers of methyl 4-chloro-2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate Intermediate 33 (240 mg) were separated by chiral chromatography on a YMC SJ (imob OJ) column (250×30 mm, 5 μm), eluted with 20% EtOH/DEA (100/20 mM) in CO₂, 150 bar, at a flow rate of 130 mL/min and detected at 220 nm;
the second eluted compound was collected and evaporated to yield the title compound Isomer 3, Intermediate 34 (50 mg); MS (ESI) m/z [M+H]⁺ 680.4; and
the third eluted compound was collected and evaporated to yield a the title compound Isomer 4, Intermediate 35 (50 mg); MS (ESI) m/z [M+H]⁺ 682.3.

### Intermediate 36

### Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate

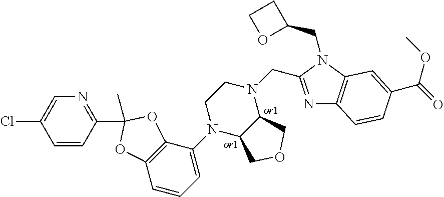 [SMILES: COC(=O)c1ccc2nc(CN3CCN(c4cccc5c4OC(C)(c4ccc(Cl)cn4)O5)[C@@H]4COC[C@@H]43)n(C[C@@H]3CCO3)c2c1]

### Step a) rac-(4aR,7aS)-1-(2-(5-Chloropyridin-2-yl)-2-methylbenzo[d][1,3]dioxol-4-yl)octahydrofuro[3,4-b]pyrazine

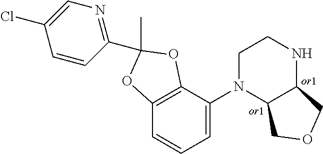 [SMILES: CC1(c2ccc(Cl)cn2)Oc2cccc(N3CCN[C@H]4COC[C@H]43)c2O1]

4-(2-(5-chloropyridin-2-yl)-2-methylbenzo[d][1,3]dioxol-4-yl)hexahydrofuro[3,4-b]pyrazine-1(2H)-carboxylate Intermediate 20 (570 mg, 1.2 mmol) in DCM (15 mL), and the reaction mixture was stirred at ambient temperature for 8 h. The reaction mixture was concentrated under reduced pressure to give the sub-title compound as an HCl salt (300 mg, 95%). MS (ESI) m/z [M+H]⁺ 374.0.

### Step b) Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benz[d]imidazole-6-carboxylate

Methyl (S)-2-(chloromethyl)-1-(oxetan-2-ylmethyl)-1H-benzo[d]imidazole-6-carboxylate (385 mg, 1.31 mmol), DIPEA (675 mg, 5.22 mmol) and NaI (587 mg, 3.92 mmol) were added to a solution of rac-(4aR,7aS)-1-(2-(5-chloropyridin-2-yl)-2-methylbenzo[d][1,3]dioxol-4-yl)octahydrofuro[3,4-b]pyrazine hydrochloride, (Step a) (488 mg, 1.31 mmol) in MeCN (5 mL) and the reaction mixture was stirred at 45° C. overnight. The reaction mixture was diluted with water, and the mixture was extracted with EtOAc. The combined organic layer was washed with sat NaCl (aq), dried over anhydrous Na₂SO₄, filtered and concentrated to dryness. The residue was purified by preparative HPLC, PrepMethod D, (gradient: 40-50%) to give the title compound (0.167 g, 43%); MS (ESI) m/z [M+H]⁺ 632.2.

### Intermediate 37

### Methyl 2-(((4aR*,7aS*)-4-(R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 1

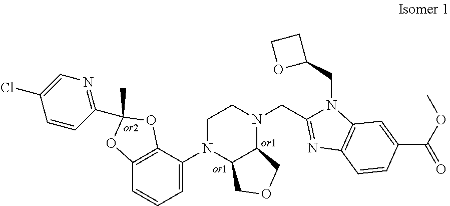 [SMILES: COC(=O)c1ccc2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 38

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 2

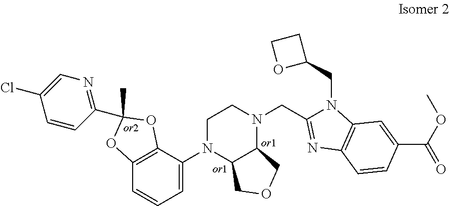 [SMILES: COC(=O)c1ccc2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 39

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 3

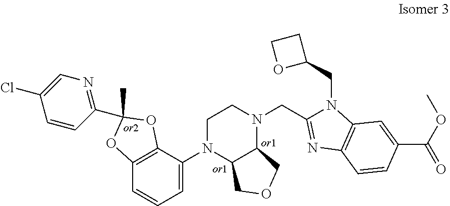 [SMILES: COC(=O)c1ccc2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 40

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 4

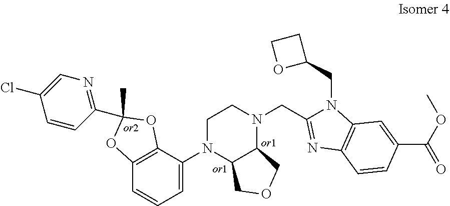 [SMILES: COC(=O)c1ccc2nc(CN3CCN(c4cccc5c4O[C@@](C)(c4ccc(Cl)cn4)O5)[C@@H]4COC[C@@H]43)n(C[C@@H]3CCO3)c2c1]

The diastereomers of methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate Intermediate 36 (167 mg) were separated by chiral chromatography on a Chiralcel OD-H column (250×20 mm, 5 μm), eluted with a mixture of hexane-IPA-MeOH (80:10:10), at a flow rate of 14 mL/min, followed by chiral chromatography on a Chiralcel OD-H column (250×20 mm, 5 μm), eluted with a mixture of hexane-IPA-MeOH (70:15:15), at a flow rate of 14 mL/min;
the first eluted compound was collected and evaporated to yield the title compound Isomer 1, Intermediate 37, MS (ESI) m/z [M+H]⁺ 632.2; and
the second eluted compound was collected and evaporated to yield the title compound Isomer 2, Intermediate 38, MS (ESI) m/z [M+H]⁺ 632.2; and
the third eluted compound was collected and evaporated to yield the title compound Isomer 3, Intermediate 39, MS (ESI) m/z [M+H]⁺ 632.2; and
the fourth eluted compound was collected and evaporated to yield the title compound Isomer 4, Intermediate 40, MS (ESI) m/z [M+H]⁺ 632.2.

### Intermediate 41

### Methyl 2-(((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate

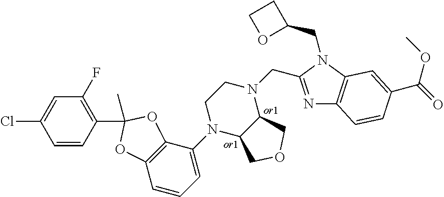 [SMILES: COC(=O)c1ccc2nc(CN3CCN(c4cccc5c4OC(C)(c4ccc(Cl)cc4F)O5)[C@@H]4COC[C@@H]43)n(C[C@@H]3CCO3)c2c1]

Methyl (S)-2-(chloromethyl)-1-(oxetan-2-ylmethyl)-1H-benzo[d]imidazole-6-carboxylate (91 mg, 308 μmol) and DIPEA (139 mg, 1.08 mmol) were added to a solution of rac-(4aR,7aS)-1-(2-(4-chloro-2-fluorophenyl)-2-methylbenzo[d][1,3]dioxol-4-yl)octahydrofuro[3,4-b]pyrazine hydrochloride Intermediate 22 (120 mg, 308 μmol) in MeCN (5 mL) and the reaction mixture was stirred at 45° C. overnight. Water was added to the reaction mixture, and the mixture was extracted with EtOAc. The combined organic layer was washed with sat NaCl (aq), dried over anhydrous Na₂SO₄, filtered and concentrated to dryness. The residue was purified by preparative HPLC, PrepMethod D, (gradient: 10-50%) to give the title compound (0.312 g, 60%); MS (ESI) m/z [M+H]⁺ 649.0.

### Intermediate 42

### Methyl 2-(((4aR*,7aS*)-4-(R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 1

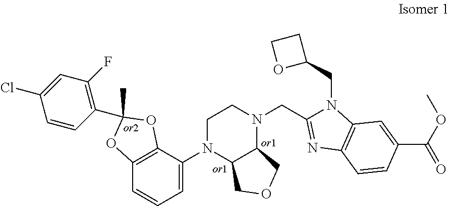 [SMILES: COC(=O)c1ccc2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 43

### Methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 2

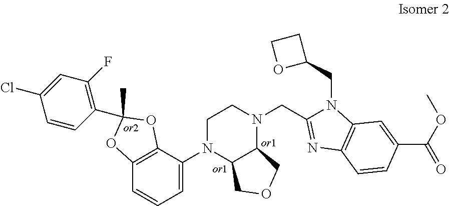 [SMILES: COC(=O)c1ccc2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 44

### Methyl 2-((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 3

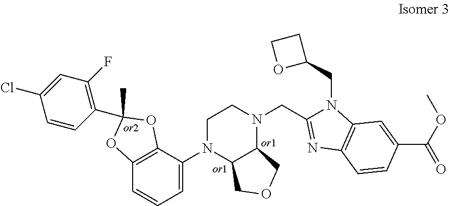 [SMILES: COC(=O)c1ccc2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 45

### Methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 4

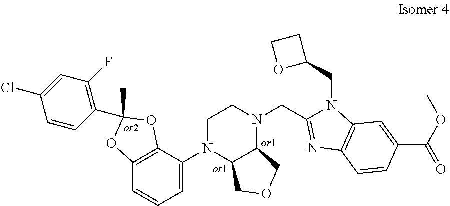 [SMILES: COC(=O)c1ccc2nc(CN3CCN(c4cccc5c4O[C@@](C)(c4ccc(Cl)cc4F)O5)[C@@H]4COC[C@@H]43)n(C[C@@H]3CCO3)c2c1]

The diastereomers of methyl 2-(((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate Intermediate 41 were separated by chiral chromatography on a Chiralpak IA column (250×4.6 mm, 5 μm) eluted with a mixture of hexane-IPA-MeOH (70:15:15) at a flow rate of 0.6 mL/min;
the first eluted compound mixture was collected and evaporated to give a mixture of two isomers and;
the second eluted compound was collected and evaporated to give the title compound Isomer 3, Intermediate 44 (29 mg); MS (ESI) m/z [M+H]⁺ 649.4;
the third eluted compound was collected and evaporated to give the title compound Isomer 4, Intermediate 45 (24 mg); MS (ESI) m/z [M+H]⁺ 649.4;

The stereoisomers of the first eluted compound mixture were separated by chiral chromatography on a Chiralpak IB column (250×4.6 mm, 5 μm) eluted with a mixture of hexane-IPA-MeOH (70:15:15) at a flow rate of 0.6 mL/min;
the first eluted compound was collected and evaporated to give the title compound Isomer 1, Intermediate 42 (38 mg); MS (ESI) m/z [M+H]⁺ 649.4;
the second eluted compound was collected and evaporated to give the title compound Isomer 2, Intermediate 43 (27 mg); MS (ESI) m/z [M+H]⁺ 649.4.

### Intermediate 46

### rac-Methyl 1-(2-(1H-pyrazol-1-yl)ethyl)-2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate

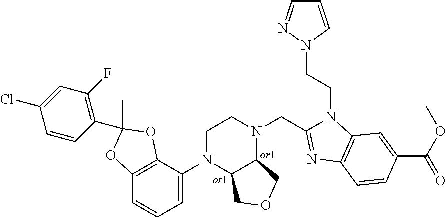 [SMILES: COC(=O)c1ccc2nc(CN3CCN(c4cccc5c4OC(C)(c4ccc(Cl)cc4F)O5)[C@@H]4COC[C@@H]43)n(CCn3cccn3)c2c1]

### Step a) Methyl 1-(2-(1H-pyrazol-1-yl)ethyl)-2-(chloromethyl)-1H-benzo[d]imidazole-6-carboxylate

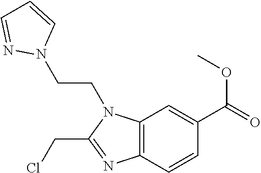 [SMILES: COC(=O)c1ccc2nc(CCl)n(CCn3cccn3)c2c1]

A solution of 2-chloro-1,1,1-trimethoxyethane (155 μL, 1.15 mmol) in MeCN (1 mL) was added to a solution of methyl 3-((2-(1H-pyrazol-1-yl)ethyl)amino)-4-aminobenzoate Intermediate 13 (284 mg, 1.09 mmol) and pTsOH (21 mg, 0.11 mmol) in MeCN (4 mL) and the reaction mixture was stirred at 50° C. for 2 h.

### Step b) rac-methyl 1-(2-(1H-pyrazol-1-yl)ethyl)-2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate

K₂CO₃ (498 mg, 3.60 mmol) and a solution of rac-(4aR,7aS)-1-(2-(4-chloro-2-fluorophenyl)-2-methylbenzo[d][1,3]dioxol-4-yl)octahydrofuro[3,4-b]pyrazine dihydrochloride Intermediate 22 (412 mg, 0.89 mmol) in MeCN (3 mL) were added to the reaction mixture of step a and the reaction mixture was stirred at 50° C. overnight. The reaction mixture was concentrated, and the residue was dissolved in EtOAc (100 mL) and washed with water (50 mL) and brine (50 mL). The organic layer was dried over MgSO₄, filtered and concentrated. The residue was purified by straight phase flash chromatography on silica (50-100% EtOAc in heptane), to give the title compound (0.360 g, 60%) as a brown oil; MS (ESI) m/z [M+H]⁺ 673.54.

### Intermediate 47

### rel-Methyl 1-(2-(1H-pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate, Isomer 1

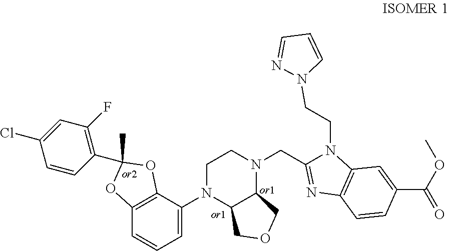 [SMILES: COC(=O)c1ccc2nc(CN3CCN(c4cccc5c4O[C@@](C)(c4ccc(Cl)cc4F)O5)[C@@H]4COC[C@@H]43)n(CCn3cccn3)c2c1]

### Intermediate 48

### rel-Methyl 1-(2-(1H-pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate, Isomer 2

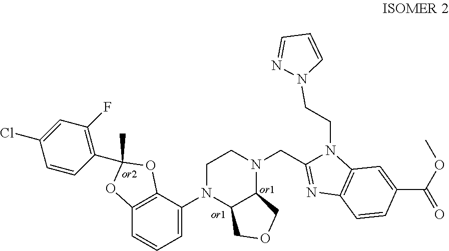 [SMILES: COC(=O)c1ccc2nc(CN3CCN(c4cccc5c4O[C@@](C)(c4ccc(Cl)cc4F)O5)[C@@H]4COC[C@@H]43)n(CCn3cccn3)c2c1]

### Intermediate 49

### rel-Methyl 1-(2-(1H-pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate, Isomer 3

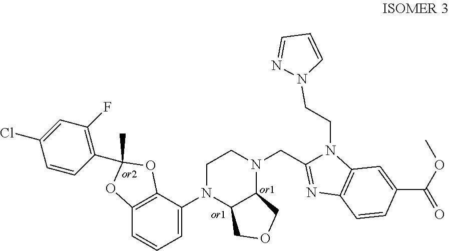 [SMILES: COC(=O)c1ccc2nc(CN3CCN(c4cccc5c4O[C@@](C)(c4ccc(Cl)cc4F)O5)[C@@H]4COC[C@@H]43)n(CCn3cccn3)c2c1]

The diastereomers of rac-methyl 1-(2-(1H-pyrazol-1-yl)ethyl)-2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate Intermediate 46 (360 mg) were separated by chiral chromatography on a Lux C4 column (250×50 mm, 5 μm), eluted with 25% EtOH/DEA (100/20 mM) in CO₂, 120 bar, at a flow rate of 400 mL/min and detected at 210 nm;
the first eluted compound was collected and evaporated to yield the title compound Isomer 1, Intermediate 47 (128 mg); MS (ESI) m/z [M+H]⁺ 673.4;
the second eluted compound was collected and evaporated to yield the title compound Isomer 2, Intermediate 48 (35 mg); MS (ESI) m/z [M+H]⁺ 673.4;
the third eluted compound mixture was collected and evaporated to yield a mixture of isomers. The isomers were separated by chiral chromatography on a Chiralpak IH column (250×30 mm, 5 μm), eluted with 8% MeOH/DEA (100/20 mM) in CO₂, 130 bar, at a flow rate of 130 mL/min and detected at 220 nm;
the first eluted compound was collected and evaporated to yield the title compound Isomer 3, Intermediate 49 (20 mg); MS (ESI) m/z [M+H]⁺ 673.3.

### Intermediate 50

### Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate

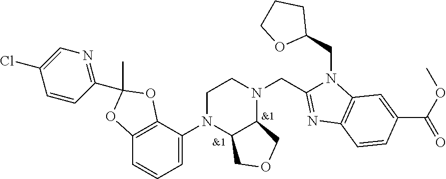 [SMILES: COC(=O)c1ccc2nc(CN3CCN(c4cccc5c4OC(C)(c4ccc(Cl)cn4)O5)[C@@H]4COC[C@@H]43)n(C[C@@H]3CCCO3)c2c1]

### Step a) rac-(4aR,7aS)-1-(2-(5-Chloropyridin-2-yl)-2-methylbenzo[d][1,3]dioxol-4-yl)octahydrofuro[3,4-b]pyrazine

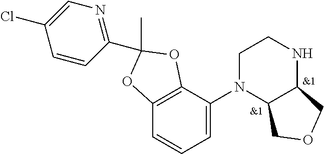 [SMILES: CC1(c2ccc(Cl)cn2)Oc2cccc(N3CCN[C@H]4COC[C@H]43)c2O1]

TFA (374 μL, 4.85 mmol) was added to a solution of rac-tert-butyl (4aR,7aS)-4-(2-(5-chloropyridin-2-yl)-2-methylbenzo[d][1,3]dioxol-4-yl)hexahydrofuro[3,4-b]pyrazine-1(2H)-carboxylate Intermediate 20 (460 mg, 0.97 mmol) in DCM (3.0 mL), and the reaction mixture was stirred at rt for 1 h. The reaction mixture was evaporated and the residue was used directly in the next step

### Step b) Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H-yl)methyl)-1-(((S)-tetrahydrofuran-2-yl)methyl)-1H-benzo[d]imidazole-6-carboxylate

The crude product from step a was dissolved in MeCN (3 mL) and K₂CO₃ (402 mg, 2.91 mmol) and methyl (S)-2-(chloromethyl)-1-((tetrahydrofuran-2-yl)methyl)-1H-benzo[d]imidazole-6-carboxylate Intermediate 16 (330 mg, 1.07 mmol) were added and the reaction mixture was heated at 60° C. for 18 h. The reaction mixture was allowed to cool to rt, and filtered through a pad of Celite®. The filtrate was concentrated and the residue was purified by flash chromatography on silica (Gradient: 0-100% EtOAc in heptane) to give the title compound (0.440 g, 70%); MS (ESI) m/z [M+H]⁺ 646.4.

### Intermediate 51

### Methyl 2-((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate, Isomer 3

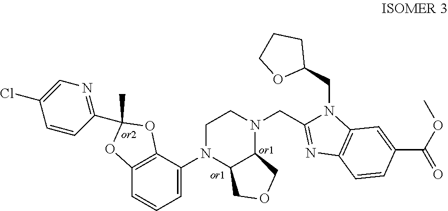 [SMILES: COC(=O)c1ccc2nc(CN3CCN(c4cccc5c4O[C@@](C)(c4ccc(Cl)cn4)O5)[C@@H]4COC[C@@H]43)n(C[C@@H]3CCCO3)c2c1]

### Intermediate 52

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate, Isomer 4

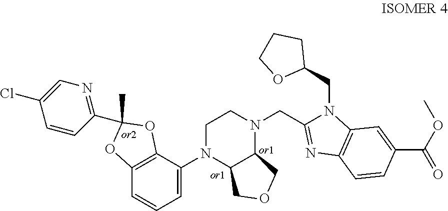 [SMILES: COC(=O)c1ccc2nc(CN3CCN(c4cccc5c4O[C@@](C)(c4ccc(Cl)cn4)O5)[C@@H]4COC[C@@H]43)n(C[C@@H]3CCCO3)c2c1]

The diastereoisomers of methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate Intermediate 50 (440 mg) were separated by chiral chromatography on a YMC SA (IA) column (250×30 mm, 5 μm), eluted with 25% EtOH/DEA (100/20 mM) in CO₂, 130 bar, at a flow rate of 150 mL/min and detected at 220 nm;
the third eluted compound mixture was collected and evaporated to yield a mixture of isomers. The isomers were separated by chiral chromatography on a Chiralcel OJ column (250×50 mm, 5 μm), eluted with 8% MeOH/DEA (100/20 mM) in CO₂, at 130 bar, at a flow rate of 350 mL/min, and detected at 220 nm;
the first eluted compound was collected and evaporated to yield the title compound Isomer 3, Intermediate 51 (94 mg); MS (ESI) m/z [M+H]⁺ 646.4;
the second eluted compound was collected and evaporated to yield the title compound Isomer 4, Intermediate 52 (60 mg); MS (ESI) m/z [M+H]⁺ 646.52.

### Intermediate 53

### Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate

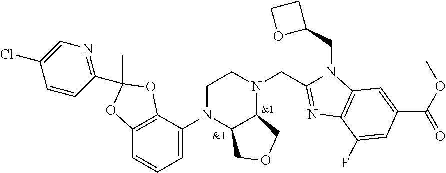 [SMILES: COC(=O)c1cc(F)c2nc(CN3CCN(c4cccc5c4OC(C)(c4ccc(Cl)cn4)O5)[C@@H]4COC[C@@H]43)n(C[C@@H]3CCO3)c2c1]

Methyl (S)-2-(chloromethyl)-4-fluoro-1-(oxetan-2-ylmethyl)-1H-benzo[d]imidazole-6-carboxylate Intermediate 19 (337 mg, 1.08 mmol), Cs₂CO₃ (1.22 g, 3.7 mmol) and NaI (485 mg, 3.24 mmol) were added to a solution of rac-(4aR,7aS)-1-(2-(5-chloropyridin-2-yl)-2-methylbenzo[d][1,3]dioxol-4-yl)octahydrofuro[3,4-b]pyrazine (Intermediate 36, Step a) (483 mg, 1.29 mmol) in DMF (7 mL) and the reaction mixture was stirred at 60° C. overnight. Water was added to the reaction mixture and the mixture was extracted with EtOAc. The combined organic layer was washed with sat NaCl (aq), dried over anhydrous Na₂SO₄, filtered and concentrated to dryness. The residue was purified by preparative HPLC, PrepMethod D, (gradient: 40-65%), to give the title compound (158 mg, 51% yield) as a mixture of isomers; MS (ESI) m/z [M+H]⁺ 650.2.

### Intermediate 54

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 1

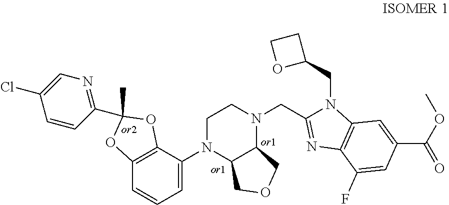 [SMILES: COC(=O)c1cc(F)c2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 55

### Methyl 2-((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 2

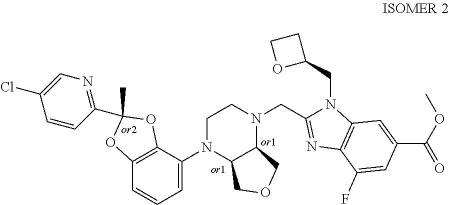 [SMILES: COC(=O)c1cc(F)c2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 56

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 3

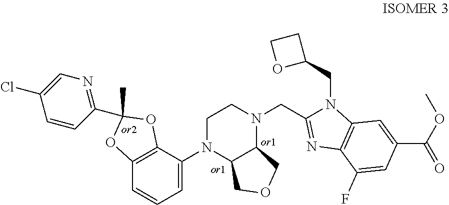 [SMILES: COC(=O)c1cc(F)c2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 57

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 4

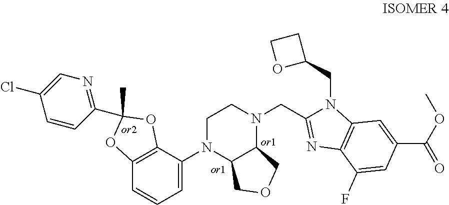 [SMILES: COC(=O)c1cc(F)c2nc(CN3CCN(c4cccc5c4O[C@@](C)(c4ccc(Cl)cn4)O5)[C@@H]4COC[C@@H]43)n(C[C@@H]3CCO3)c2c1]

The diastereoisomers of methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate Intermediate 53 were separated by chiral chromatography on a Chiral ART Cellulose-SC column (250×20 mm, 5 μm), eluted with Hexane/IPA/MeOH (80:10:10), at a flow rate of 12 mL/min;
the first eluted compound was collected and evaporated to yield the title compound Isomer 1, Intermediate 54 (29 mg); MS (ESI) m/z [M+H]⁺ 650.2;
the second eluted compound was collected and evaporated to yield the title compound Isomer 2, Intermediate 55 (21 mg); MS (ESI) m/z [M+H]⁺ 650.2;
the third eluted compound was collected and evaporated to yield the title compound Isomer 3, Intermediate 56 (36 mg); MS (ESI) m/z [M+H]⁺ 650.2;
the fourth eluted compound was collected and evaporated to yield the title compound Isomer 4, Intermediate 57 (22 mg); MS (ESI) m/z [M+H]⁺ 650.2.

### Intermediate 58

### tert-Butyl (4aS,7aR)-4-(2-(4-chloro-2-fluorophenyl)-2-methylbenzo[d][1,3]dioxol-4-yl)hexahydrofuro[3,4-b]pyrazine-1(2H)-carboxylate

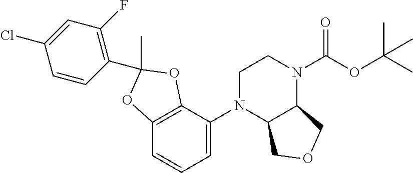 [SMILES: CC(C)(C)OC(=O)N1CCN(c2cccc3c2OC(C)(c2ccc(Cl)cc2F)O3)[C@@H]2COC[C@@H]21]

Cs₂CO₃ (856 mg, 2.63 mmol) was added to a mixture of 4-bromo-2-(4-chloro-2-fluorophenyl)-2-methylbenzo[d][1,3]dioxole WO2020234726 (361 mg, 1.05 mmol), tert-butyl (4aS,7aR)-hexahydrofuro[3,4-b]pyrazine-1(2H)-carboxylate Intermediate 163 (200 mg, 0.88 mmol), 2′-(bis(3,5-bis(trifluoromethyl)phenyl)phosphino)-3′,6′-dimethoxy-N2,N2,N6,N6-tetramethyl-[1,1′-biphenyl]-2,6-diamine (14 mg, 0.04 mmol) and methanesulfonato(2-bis(3,5-di(trifluoromethyl)phenylphosphino)-3,6-dimethoxy-2′,6′-bis(dimethylamino)-1,1′-biphenyl)(2′-methylamino-1,1′-biphenyl-2-yl)palladium(II) (50 mg, 0.04 mmol) in 1,4-dioxane (4 mL) at 30° C. under an atmosphere of N₂ (g), and the reaction mixture was stirred at 90° C. for 16 h. Another batch was prepared as described above and the reaction mixtures were combined. The combined reaction mixture was diluted with EtOAc (100 mL) and washed sequentially with sat brine (100 mL) and water (100 mL). The organic layer was dried over Na₂SO₄, filtered and evaporated. The crude product was purified by preparative TLC (petroleum ether:EtOAc, 3:1) to give the title compound (0.890 g, 83%) as a yellow oil which solidified upon standing; MS (ES+) m/z [M+H]⁺ 491.

### Intermediate 59

### (4aR,7aS)-1-(2-(4-Chloro-2-fluorophenyl)-2-methylbenzo[d][1,3]dioxol-4-yl)octahydrofuro[3,4-b]pyrazine

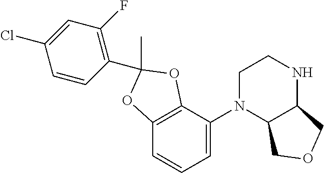 [SMILES: CC1(c2ccc(Cl)cc2F)Oc2cccc(N3CCN[C@H]4COC[C@H]43)c2O1]

tert-Butyl (4aS,7aR)-4-(2-(4-chloro-2-fluorophenyl)-2-methylbenzo[d][1,3]dioxol-4-yl)hexahydrofuro[3,4-b]pyrazine-1(2H)-carboxylate Intermediate 58 (870 mg, 1.77 mmol) was dissolved in DCM (30 mL) and TFA (10 mL) at 25° C. and the reaction mixture was stirred at 25° C. for 3 h. The solvent was removed under reduced pressure and the crude product was precipitated from EtOAc. The suspension was filtered and the solids were collected and dried under vacuum to give the title compound as a TFA salt (890 mg, 99%); MS (ESI) m/z [M+H]⁺ 391.

### Intermediate 60

### Methyl 1-(2-(1H-pyrazol-1-yl)ethyl)-2-(chloromethyl)-1H-benzo[d]imidazole-6-carboxylate

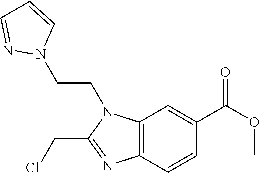 [SMILES: COC(=O)c1ccc2nc(CCl)n(CCn3cccn3)c2c1]

2-Chloro-1,1,1-trimethoxyethane (0.75 mL, 5.58 mmol) was added to a solution of methyl 3-((2-(1H-pyrazol-1-yl)ethyl)amino)-4-aminobenzoate Intermediate 13 (1.09 g, 4.19 mmol) and pTsOH hydrate (0.086 g, 0.45 mmol) in MeCN (20 mL) and the reaction mixture was stirred at 50° C. for 1.5 h. Additional 2-chloro-1,1,1-trimethoxyethane (0.2 mL, 1.49 mmol) was added and the mixture was heated for 1 h. EtOAc (200 mL) was added and the mixture was washed with sat NaHCO₃ (aq, 2×50 mL). The organic layer was dried over MgSO₄, filtered, and concentrated to give the title compound (1.27 g, 95%) as a brown solid; MS (ESI) m/z [M+H]⁺ 319.2.

### Intermediate 61

### N-(2-(1H-Pyrazol-1-yl)ethyl)-5-bromo-3-fluoro-2-nitroaniline

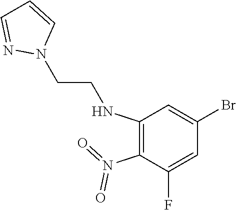 [SMILES: O=[N+]([O-])c1c(F)cc(Br)cc1NCCn1cccn1]

2-(1H-Pyrazol-1-yl)ethan-1-amine (0.934 g, 8.40 mmol) was added to 5-bromo-1,3-difluoro-2-nitrobenzene (2.0 g, 8.40 mmol) and DIPEA (3.67 mL, 21.01 mmol) in MeCN (25 mL) at 20° C. and the reaction mixture was stirred at 22° C. for 1 h. The reaction mixture was diluted with EtOAc (100 mL) and washed with sat brine (75 mL). The organic layer was dried over Na₂SO₄, filtered and evaporated. The crude product was purified by preparative TLC (EtOAc:petroleum ether, 5:1), to afford the title compound (2.40 g, 87%) as a yellow solid; MS (ESI) m/z [M+H]⁺ 329/331.

### Intermediate 62

### N1-(2-(1H-Pyrazol-1-yl)ethyl)-5-bromo-3-fluorobenzene-1,2-diamine

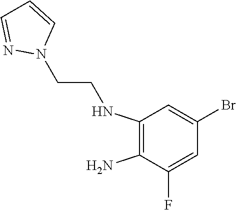 [SMILES: Nc1c(F)cc(Br)cc1NCCn1cccn1]

Zn(s) (3.66 g, 55.91 mmol) was added to N-(2-(1H-pyrazol-1-yl)ethyl)-5-bromo-3-fluoro-2-nitroaniline Intermediate 61 (2.3 g, 6.99 mmol) and NH₄Cl (3.74 g, 69.88 mmol) in MeOH (80 mL) and water (20 mL) at 20° C., and the reaction mixture was stirred at 60° C. for 2 h. The reaction mixture was filtered through Celite and the filter cake was washed with MeOH (100 mL). The combined filtrate was concentrated at reduced pressure. The residue was diluted with DCM (100 mL) and washed sequentially with sat brine (100 mL) and water. The organic layer was dried over Na₂SO₄, filtered and concentrated. The crude product was purified by preparative TLC (MeOH:DCM, 4:1) to give the title product (1.50 g, 72%) as a white solid; MS (ESI) m/z [M+H]⁺ 299/300.

### Intermediate 63

### Methyl 3-((2-(1H-pyrazol-1-yl)ethyl)amino)-4-amino-5-fluorobenzoate

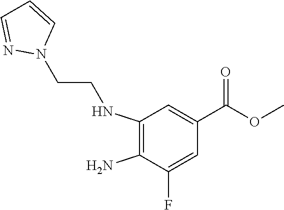 [SMILES: COC(=O)c1cc(F)c(N)c(NCCn2cccn2)c1]

N1-(2-(1H-Pyrazol-1-yl)ethyl)-5-bromo-3-fluorobenzene-1,2-diamine Intermediate 62 (0.5 g, 1.67 mmol), Pd(dppf)Cl₂ (0.122 g, 0.17 mmol) and DIPEA (2.92 mL, 16.71 mmol) in MeOH (150 mL) was stirred under an atmosphere of CO (g) at 60 atm and 120° C. for 12 h. The reaction mixture was concentrated and diluted with EtOAc (150 mL) and washed with sat brine (75 mL). The organic layer was dried over Na₂SO₄, filtered and evaporated. The crude product was purified by preparative TLC (EtOAc:petroleum ether, 2:1) to give the title compound (0.30 g, 64%) as a yellow solid; MS (ESI) m/z [M+H]⁺ 279.0.

### Intermediate 64

### Methyl 1-(2-(1H-pyrazol-1-yl)ethyl)-2-(chloromethyl)-4-fluoro-1H-benzo[d]imidazole-6-carboxylate

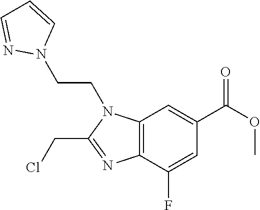 [SMILES: COC(=O)c1cc(F)c2nc(CCl)n(CCn3cccn3)c2c1]

2-Chloro-1,1,1-trimethoxyethane (0.70 mL, 5.21 mmol) was added to a solution of methyl 3-((2-(1H-pyrazol-1-yl)ethyl)amino)-4-amino-5-fluorobenzoate Intermediate 63 (1.06 g, 3.81 mmol) and pTsOH hydrate (0.086 g, 0.45 mmol) in MeCN (20 mL) and the resulting mixture was stirred at 50° C. for 2 h. EtOAc (100 mL) was added and the mixture was washed with sat NaHCO₃ (aq, 2×50 mL). The combined organic layer was dried over MgSO₄, filtered, and the solvent was removed at reduced pressure to give the title compound (1.23 g, 96%) as a brown solid; MS (ESI) m/z [M+H]⁺ 377.0.

### Intermediate 65

### Methyl (S)-2-(chloromethyl)-4-fluoro-1-((tetrahydrofuran-2-yl)methyl)-1H-benzo[d]imidazole-6-carboxylate

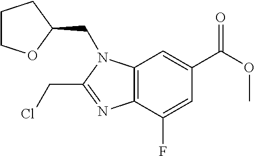 [SMILES: COC(=O)c1cc(F)c2nc(CCl)n(C[C@@H]3CCCO3)c2c1]

The title compound was prepared from 5-bromo-1,3-difluoro-2-nitrobenzene, in 4 steps, in analogy with the description for Intermediate 7 with the exception that in step 3 a solvent mixture of MeOH:DMSO (2:1) was used. The preparation gave the title compound (500 mg, 93%); MS (ESI) m/z [M+H]⁺ 327.2.

### Intermediate 66

### Methyl 3-fluoro-4-nitro-5-((pyridin-3-ylmethyl)amino)benzoate

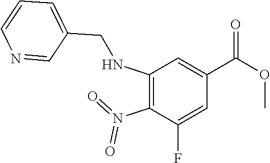 [SMILES: COC(=O)c1cc(F)c([N+](=O)[O-])c(NCc2cccnc2)c1]

A mixture of methyl 3,5-difluoro-4-nitrobenzoate (2.0 g, 9.21 mmol), (pyridin-3-yl)methanamine (996 mg, 9.21 mmol) and DIPEA (5.95 g, 46.06 mmol) in THF (10 mL) was stirred at 45° C. for 16 h, and then cooled to rt. The mixture was diluted with water and extracted with MTBE. The combined organic layer was washed with brine, dried over anhydrous Na₂SO₄, filtered, and concentrated to give the title compound (2.5 g, 89%); MS (ESI) m/z [M+H]⁺ 306.2.

### Intermediate 67

### Methyl 4-amino-3-fluoro-5-((pyridin-3-ylmethyl)amino)benzoate

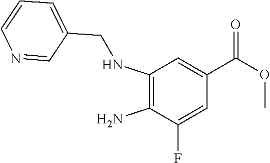 [SMILES: COC(=O)c1cc(F)c(N)c(NCc2cccnc2)c1]

Methyl 3-fluoro-4-nitro-5-((pyridin-3-yl)methyl)aminobenzoate Intermediate 66 (2.5 g, 8.19 mmol) was dissolved in MeOH (15 mL) and treated with Pd/C (10%, 250 mg). The resulting mixture was hydrogenated under an atmosphere of H₂ (g) at ambient pressure and temperature until the reaction was complete. The reaction mixture was filtered and the filtrate was collected and concentrated. The residue was purified by flash chromatography on silica (DCM:MeCN, 1:1) to give the title compound (1.25 g, 53%); ¹H NMR (500 MHz, DMSO-d₆) δ 8.58 (d, 1H), 8.45 (dd, 1H), 7.74 (dt, 1H), 7.35 (dd, 1H), 6.99 (dd, 1H), 6.83 (d, 1H), 5.64 (t, 1H), 5.43 (s, 2H), 4.36 (d, 2H), 3.69 (d, 3H).

### Intermediate 68

### Methyl 2-(chloromethyl)-4-fluoro-1-(pyridin-3-ylmethyl)-1H-benzo[d]imidazole-6-carboxylate

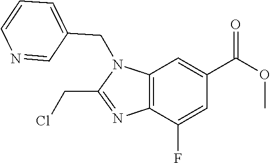 [SMILES: COC(=O)c1cc(F)c2nc(CCl)n(Cc3cccnc3)c2c1]

Methyl 4-amino-3-fluoro-5-((pyridin-3-yl)methyl)aminobenzoate Intermediate 67 (600 mg, 2.18 mmol) was dissolved in THF (10 mL). 2-Chloro-1,1,1-trimethoxyethane (404 mg, 2.61 mmol) and pTsOH hydrate (41 mg, 218 μmol) were added, and the reaction mixture was stirred at 50° C. overnight. 4 M HCl in 1,4-dioxane (15 mL) was added and the reaction mixture was filtered. The solids were collected and dried in vacuo to give the HCl salt of the title compound (700 mg, 96%); MS (ESI) m/z [M+H]⁺ 334.0.

### Intermediate 69

### (S)-5-Bromo-3-chloro-2-nitro-N-(oxetan-2-ylmethyl)aniline

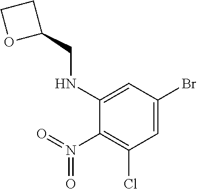 [SMILES: O=[N+]([O-])c1c(Cl)cc(Br)cc1NC[C@@H]1CCO1]

DIPEA (15.44 mL, 88.43 mmol) and 5-bromo-1-chloro-3-fluoro-2-nitrobenzene (7.5 g, 29.48 mmol) were added dropwise to a solution of (S)-oxetan-2-ylmethanamine (2.57 g, 29.48 mmol) in THF (200 mL), and the reaction mixture was stirred at 60° C. for 3 h. The reaction mixture was diluted with EtOAc (300 mL) and washed with sat brine (4×300 mL). The organic layer was dried over Na₂SO₄, filtered and evaporated. The crude product was purified by flash chromatography on silica (0-50% EtOAc in petroleum ether) to give the title compound (9.00 g, 95%) as a yellow oil; MS (ESI) m/z [M+H]⁺ 321/323.

### Intermediate 70

### (S)-5-Bromo-3-chloro-N1-(oxetan-2-ylmethyl)benzene-1,2-diamine

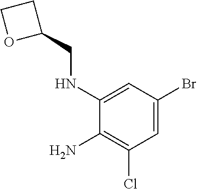 [SMILES: Nc1c(Cl)cc(Br)cc1NC[C@@H]1CCO1]

Fe(s) (24.66 g, 441.60 mmol) was added to a mixture of (S)-5-bromo-3-chloro-2-nitro-N-(oxetan-2-ylmethyl)aniline Intermediate 69 (14.2 g, 44.16 mmol) and NH₄Cl (23.62 g, 441.60 mmol) in MeOH (400 mL) and water (100 mL) at 20° C., and the reaction mixture was stirred at 60° C. for 6 h. The reaction mixture was filtered and the precipitate was washed with MeOH (4×100 mL). The filtrate was concentrated under reduced pressure and the crude product was diluted with EtOAc (500 mL). The organic layer was washed sequentially with water (500 mL) and sat brine (500 mL), dried over Na₂SO₄, filtered and evaporated. The crude product was purified by flash chromatography on silica (30-50% EtOAc in petroleum ether) to give the title compound (12.0 g, 93%) as a white solid; MS (ESI) m/z [M+H]⁺ 292/291.

### Intermediate 71

### Methyl (S)-4-amino-3-chloro-5-((oxetan-2-ylmethyl)amino)benzoate

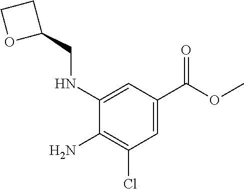 [SMILES: COC(=O)c1cc(Cl)c(N)c(NC[C@@H]2CCO2)c1]

A mixture of (S)-5-bromo-3-chloro-N1-(oxetan-2-ylmethyl)benzene-1,2-diamine Intermediate 70 (1.5 g, 5.14 mmol), Pd(dppf)Cl₂·DCM (0.38 g, 0.51 mmol) and DIPEA (8.99 mL, 51.45 mmol) in MeOH (300 mL) was stirred under an atmosphere of CO (g) at 60 atm and 120° C. for 30 h. The solvent was removed under reduced pressure and the crude product was purified by flash chromatography on silica (20-25% EtOAc in petroleum ether) to give the title compound (1.0 g, 72%) as a white solid; MS (ESI) m/z [M+H]⁺ 271.

### Intermediate 72

### Methyl (S)-4-chloro-2-(chloromethyl)-1-(oxetan-2-ylmethyl)-1H-benzo[d]imidazole-6-carboxylate

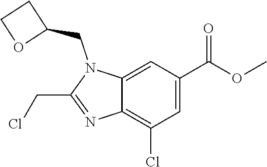 [SMILES: COC(=O)c1cc(Cl)c2nc(CCl)n(C[C@@H]3CCO3)c2c1]

pTsOH (0.357 g, 1.88 mmol) was added to a solution of methyl (S)-4-amino-3-chloro-5-((oxetan-2-ylmethyl)amino)benzoate Intermediate 71 (5.08 g, 18.77 mmol) and 2-chloro-1,1,1-trimethoxyethane (3.77 g, 24.40 mmol) in MeCN (20 mL) and the reaction mixture was stirred at 50° C. for 30 min. 2-Chloro-1,1,1-trimethoxyethane (1.16 g, 7.51 mmol) was added and the reaction mixture was stirred at 50° C. for 20 min. The reaction mixture was diluted with EtOAc (10 mL) and washed with NaHCO₃ (aq, 2×3 mL). The organic layer was dried over MgSO₄, filtered and concentrated at reduced pressure. The crude compound was purified by flash chromatography on silica (50-100% EtOAc in heptane) to give the title compound (5.30 g, 86%); MS (ESI) m/z [M+H]⁺ 329.1.

### Intermediate 73

### Methyl 3-fluoro-5-((2-(2-methyl-1H-imidazol-1-yl)ethyl)amino)-4-nitrobenzoate

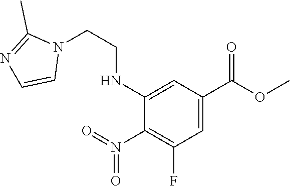 [SMILES: COC(=O)c1cc(F)c([N+](=O)[O-])c(NCCn2ccnc2C)c1]

A mixture of methyl 3,5-difluoro-4-nitrobenzoate (3.0 g, 13.82 mmol), 2-(2-methyl-1H-imidazol-1-yl)ethan-1-amine (1.73 g, 13.82 mmol) and DIPEA (8.93 g, 69.08 mmol) in THE (15 mL) was stirred at 45° C. for 16 h, and then cooled to rt. The mixture was diluted with water and extracted with MTBE. The combined organic layer was washed with brine, dried over anhydrous Na₂SO₄, and filtered. The filtrate was concentrated under reduced pressure to give the title compound (3.5 g, 57%); MS (ESI) m/z [M+H]⁺ 323.0.

### Intermediate 74

### Methyl 4-amino-3-fluoro-5-((2-(2-methyl-1H-imidazol-1-yl)ethyl)amino)benzoate

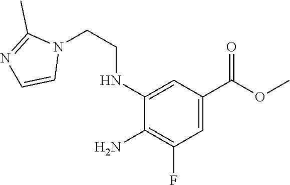 [SMILES: COC(=O)c1cc(F)c(N)c(NCCn2ccnc2C)c1]

Methyl 3-fluoro-5-(2-(2-methyl-1H-imidazol-1-yl)ethyl)amino-4-nitrobenzoate Intermediate 73 (3.5 g, 10.9 mmol) was dissolved in MeOH (15 mL) and treated with Pd/C (10%, 350 mg). The reaction mixture was hydrogenated under an atmosphere of H₂ (g) at ambient pressure and temperature until the reaction was complete. The reaction mixture was filtered, and the filtrate was collected and concentrated. The residue was purified by flash chromatography on silica (0-95% MeOH in MeCN) to give the title compound (1.4 g, 44%); ¹H NMR (400 MHz, DMSO-d₆) δ 7.19 (t, 1H), 7.08-6.97 (m, 1H), 6.89 (d, 1H), 6.81 (d, 1H), 5.39 (s, 2H), 5.29 (d, 1H), 4.14-4.04 (m, 2H), 3.75 (d, 3H), 3.42 (q, 2H), 2.27 (d, 3H).

### Intermediate 75

### Methyl 4-fluoro-2-(hydroxymethyl)-1-(2-(2-methyl-1H-imidazol-1-yl)ethyl)-1H-benzo[d]imidazole-6-carboxylate

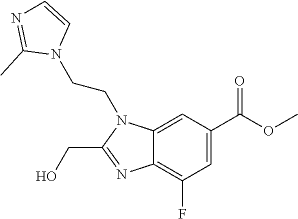

Methyl 4-amino-3-fluoro-5-(2-(2-methyl-1H-imidazol-1-yl)ethyl)aminobenzoate Intermediate 74 (1.4 g, 4.79 mmol) was dissolved in THF (80 mL). 2,2,2-Triethoxyethan-1-ol (2.56 g, 14.37 mmol) and pTsOH hydrate (91 mg, 479 μmol) were added, and the reaction mixture was stirred at 50° C. overnight. The mixture was poured into an excessive amount of water and extracted with EtOAc (×3). The combined organic layer was washed with brine and concentrated under vacuum to give the title compound (1.5 g, 94%); MS (ESI) m/z [M+H]⁺ 333.2.

### Intermediate 76

### Methyl 2-(chloromethyl)-4-fluoro-1-(2-(2-methyl-1H-imidazol-1-yl)ethyl)-1H-benzo[d]imidazole-6-carboxylate

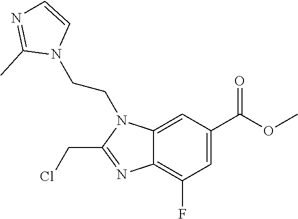

Thionyl chloride (2.68 g, 22.56 mmol) was added dropwise to a solution of methyl 4-fluoro-2-(hydroxymethyl)-1-(2-(2-methyl-1H-imidazol-1-yl)ethyl)-1H-benzo[d]imidazole-6-carboxylate Intermediate 75 (1.5 g, 4.51 mmol) in DCM (15 mL) and the reaction mixture was stirred at 50° C. for 2 h. The reaction mixture was concentrated under vacuum, and the residue was suspended in dry Et₂O (250 mL) and filtered, to give the dihydrochloride of the title compound (1.0 g, 63%); MS (ESI) m/z [M+H]⁺ 351.0.

### Intermediate 77

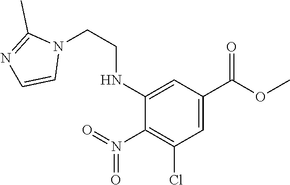 [SMILES: COC(=O)c1cc(Cl)c([N+](=O)[O-])c(NCCn2ccnc2C)c1]

### Methyl 3-chloro-5-((2-(2-methyl-1H-imidazol-1-yl)ethyl)amino)-4-nitrobenzoate

A solution of 2-(2-methyl-1H-imidazol-1-yl)ethan-1-amine dihydrochloride (2.63 g, 13.28 mmol) in dry DMF (50 mL) was slowly added to a mixture of methyl 3-chloro-5-fluoro-4-nitrobenzoate (3.1 g, 13.28 mmol) and DIPEA (6.01 g, 46.49 mmol) in dry DMF (15 mL), and the reaction mixture was stirred at 60° C. for 16 h. The reaction mixture was poured into water (150 mL) and extracted with DCM (3×60 mL). The combined organic layer was dried over Na₂SO₄, filtered, and concentrated under reduced pressure to give the title compound (3.46 g, 77% yield) as an orange oil; MS (ESI) m/z [M+H]⁺ 339.2.

### Intermediate 78

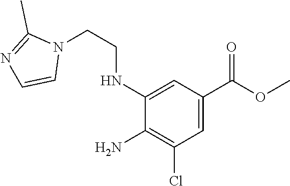 [SMILES: COC(=O)c1cc(Cl)c(N)c(NCCn2ccnc2C)c1]

### Methyl 4-amino-3-chloro-5-((2-(2-methyl-1H-imidazol-1-yl)ethyl)amino)benzoate

Pt/C (10%, 0.6 g) was added to a solution of methyl 3-chloro-5-(2-(2-methyl-1H-imidazol-1-yl)ethyl)amino-4-nitrobenzoate Intermediate 77 (3.46 g, 10.2 mmol) in MeOH (100 mL), and the reaction mixture was stirred under an atmosphere of H₂ (g) at 1 atm and rt for 5 days. The reaction mixture was filtered through a pad of Celite, and the filtrate was concentrated in vacuo. The residue was purified by chromatography on silica (10% MeOH in CHCl₃) to give the title compound (3 g, 96%) as yellow solid; ¹H NMR (400 MHz, CDCl₃) δ 7.58 (d, 1H), 7.24 (s, 2H), 6.87 (s, 1H), 6.82-6.73 (m, 1H), 4.33 (s, 2H), 4.08 (t, 2H), 3.85 (s, 3H), 3.69 (s, 1H), 3.53 (s, 2H), 2.29 (s, 2H).

### Intermediate 79

### Methyl 4-chloro-2-(hydroxymethyl)-1-(2-(2-methyl-1H-imidazol-1-yl)ethyl)-1H-benzo[d]imidazole-6-carboxylate

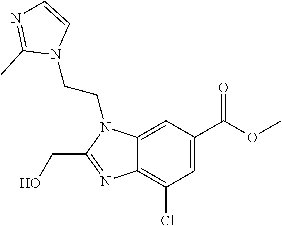

2,2,2-Triethoxyethan-1-ol (1.85 g, 10.36 mmol) and pTsOH (59 mg, 345 μmol) were added to a solution of methyl 4-amino-3-chloro-5-(2-(2-methyl-1H-imidazol-1-yl)ethyl)aminobenzoate Intermediate 78 (1.07 g, 3.45 mmol) in MeCN (7 mL) and the reaction mixture was stirred at 60° C. for 16 h. The reaction mixture was allowed to cool to rt and then concentrated in vacuo. The residue was diluted with water (50 mL), and the pH was adjusted to 7 with sat Na₂CO₃ (aq). The mixture was extracted with EtOAc (3×50 mL) and the combined organic layer was washed with brine (50 mL) and water (50 mL), dried over Na₂SO₄, filtered, and concentrated in vacuo to give the crude title compound (1.14 g); MS (ESI) m/z [M+H]⁺ 349.2.

### Intermediate 80

### Methyl 4-chloro-2-(chloromethyl)-1-(2-(2-methyl-1H-imidazol-1-yl)ethyl)-1H-benzo[d]imidazole-6-carboxylate

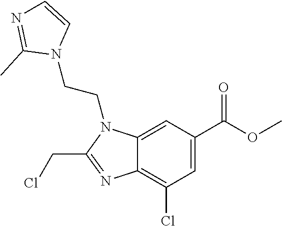

A catalytic amount of DMF followed by thionyl chloride (3.89 g, 32.68 mmol) were added dropwise to a vigorously stirred solution of methyl 4-chloro-2-(hydroxymethyl)-1-(2-(2-methyl-1H-imidazol-1-yl)ethyl)-1H-benzo[d]imidazole-6-carboxylate Intermediate 79 (1.14 g, 3.27 mmol) in DCM (15 mL). The reaction mixture was stirred at 40° C. for 16 h. The reaction mixture was concentrated in vacuo to give the HCl salt of the title compound (0.95 g, 79%) as a yellow solid; MS (ESI) m/z [M+H]⁺ 367.0.

### Intermediate 81

### Methyl 3-chloro-4-nitro-5-((oxazol-4-ylmethyl)amino)benzoate

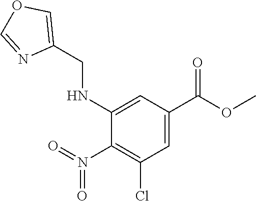 [SMILES: COC(=O)c1cc(Cl)c([N+](=O)[O-])c(NCc2cocn2)c1]

A mixture of methyl 3-chloro-5-fluoro-4-nitrobenzoate (4.2 g, 17.97 mmol), (1,3-oxazol-4-yl)methanamine hydrochloride (2.42 g, 17.97 mmol) and DIPEA (5.81 g, 44.94 mmol) in DMF (20 mL) was heated to 50° C. for 16 h. The reaction mixture was poured into water (100 mL) and filtered to give the crude title compound (4.45 g) as dark yellow solid; MS (ESI) m/z [M+H]⁺ 312.0.

### Intermediate 82

### Methyl 4-amino-3-chloro-5-((oxazol-4-ylmethyl)amino)benzoate

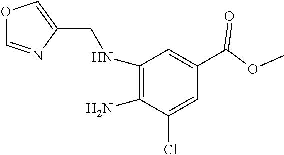 [SMILES: COC(=O)c1cc(Cl)c(N)c(NCc2cocn2)c1]

Wet Pt/C (10%, 0.8 g) was added to a yellow suspension of methyl 3-chloro-4-nitro-5-((oxazol-4-ylmethyl)amino)benzoate Intermediate 81 (4.45 g, 14.94 mmol) in MeOH (100 mL), and the reaction mixture was stirred under an atmosphere of H₂ (g) at 1 atm and 20° C. for 16 h. The reaction mixture was filtered, and the filter cake was washed with MeOH (50 mL). The filtrate was concentrated under reduced pressure and the crude product was purified by flash chromatography on silica (0-99% MTBE in hexane) to give the title compound (2.13 g, 68%); ¹H NMR (500 MHz, CDCl₃) δ 7.90 (d, 1H), 7.69-7.54 (m, 2H), 7.30 (q, 1H), 4.51-4.00 (m, 5H), 3.85 (s, 3H).

### Intermediate 83

### Methyl 4-chloro-2-(chloromethyl)-1-(oxazol-4-ylmethyl)-1H-benzo[d]imidazole-6-carboxylate

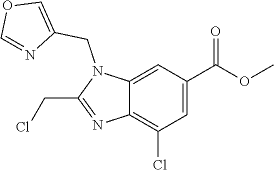 [SMILES: COC(=O)c1cc(Cl)c2nc(CCl)n(Cc3cocn3)c2c1]

2-Chloro-1,1,1-trimethoxyethane (1.36 g, 8.77 mmol) and pTsOH (137 mg, 797 μmol) were added to a stirred solution of methyl 4-amino-3-chloro-5-((oxazol-4-ylmethyl)amino)benzoate Intermediate 82 (2.13 g, 7.97 mmol) in MeCN (30 mL) and the reaction mixture was heated at 60° C. for 18 h. The reaction mixture was evaporated under reduced pressure, and the residue was extracted with EtOAc (3×20 mL). The combined organic layer was washed with NaHCO₃, dried over Na₂SO₄, filtered and evaporated. The crude product was purified by column chromatography on silica (CHCl₃:EtOAc, 60:40) to give the title compound (1.1 g, 43%); ¹H NMR (400 MHz, CDCl₃) δ 8.10-8.04 (m, 1H), 8.03-7.96 (m, 1H), 7.82 (s, 1H), 7.65 (t, 1H), 5.45 (s, 2H), 5.07 (s, 2H), 4.00-3.84 (m, 3H).

### Intermediate 84

### Methyl 3-fluoro-4-nitro-5-((oxazol-4-ylmethyl)amino)benzoate

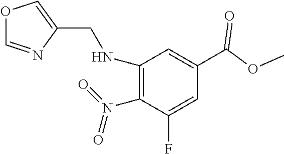 [SMILES: COC(=O)c1cc(F)c([N+](=O)[O-])c(NCc2cocn2)c1]

A mixture of methyl 3,5-difluoro-4-nitrobenzoate (1.5 g, 6.91 mmol), (1,3-oxazol-4-yl)methanamine hydrochloride (930 mg, 6.91 mmol) and DIPEA (2.68 g, 20.73 mmol, 3.61 mL) in THF (10 mL) was stirred at 45° C. for 2 h, and then cooled to rt. The mixture was diluted with water and extracted with MTBE (3×20 mL). The combined organic layer was washed with brine (50 mL), dried over anhydrous Na₂SO₄, and filtered. The filtrate was concentrated in vacuo to give the title compound (2.3 g, 79%); MS (ESI) m/z [M+H]⁺ 297.2.

### Intermediate 85

### Methyl 4-amino-3-fluoro-5-((oxazol-4-ylmethyl)amino)benzoate

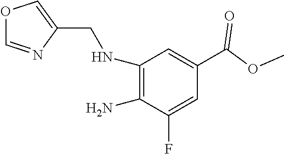 [SMILES: COC(=O)c1cc(F)c(N)c(NCc2cocn2)c1]

Methyl 3-fluoro-4-nitro-5-((oxazol-4-ylmethyl)amino)benzoate Intermediate 84 (2.3 g, 5.45 mmol) was dissolved in dry THF (10 mL) and treated with Pd/C (10%, 0.2 g). The reaction mixture was hydrogenated under an atmosphere of H₂ (g) at 1 atm and ambient temperature until the reaction was complete. The reaction mixture was filtered and the filtrate was concentrated in vacuo. The residue was purified by flash chromatography on silica (0-40% MeOH in CHCl₃) to give the title compound (1.5 g, 88%); MS (ESI) m/z [M+H]⁺ 266.0.

### Intermediate 86

### Methyl 2-(chloromethyl)-4-fluoro-1-(oxazol-4-ylmethyl)-1H-benzo[d]imidazole-6-carboxylate

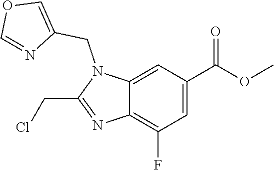 [SMILES: COC(=O)c1cc(F)c2nc(CCl)n(Cc3cocn3)c2c1]

2-Chloro-1,1,1-trimethoxyethane (1.05 g, 6.79 mmol) and pTsOH hydrate (54 mg, 283 μmol) was added to a solution of methyl 4-amino-3-fluoro-5-((oxazol-4-ylmethyl)amino)benzoate Intermediate 85 (1.5 g, 4.81 mmol) in THF (50 mL) and the reaction mixture was stirred at 50° C. overnight. The mixture was poured into water and extracted with EtOAc (3×10 mL). The combined organic layer was washed with brine and concentrated under vacuum to give the title compound (1.6 g, 60%); MS (ESI) m/z [M+H]⁺ 324.2.

### Intermediate 87

### Methyl 3-(((1-(cyanomethyl)cyclopropyl)methyl)amino)-5-fluoro-4-nitrobenzoate

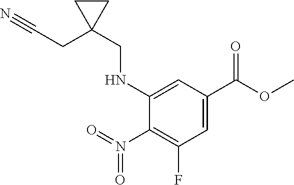 [SMILES: COC(=O)c1cc(F)c([N+](=O)[O-])c(NCC2(CC#N)CC2)c1]

A mixture of methyl 3,5-difluoro-4-nitrobenzoate (5.0 g, 23.03 mmol), 2-(1-(aminomethyl)cyclopropyl)acetonitrile hydrochloride (3.38 g, 23.03 mmol) and DIPEA (12.0 mL, 69.08 mmol) in THF (20 mL) was stirred at 60° C. for 16 h. The reaction mixture was cooled to rt and diluted with water. The aqueous phase was extracted with MTBE (3×50 mL), and the combined organic layer was washed with brine, dried over anhydrous Na₂SO₄, and filtered. The filtrate was concentrated in vacuo to give the title compound (5.1 g, 54%); MS (ESI) m/z [M+H]⁺ 308.0.

### Intermediate 88

### Methyl 4-amino-3-(((1-(cyanomethyl)cyclopropyl)methyl)amino)-5-fluorobenzoate

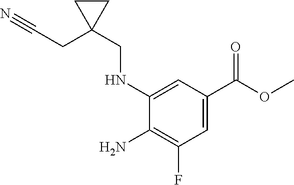 [SMILES: COC(=O)c1cc(F)c(N)c(NCC2(CC#N)CC2)c1]

Pd/C (10%, 0.5 g) was added to a solution of methyl 3-(((1-(cyanomethyl)cyclopropyl)methyl)amino)-5-fluoro-4-nitrobenzoate Intermediate 87 (5.1 g, 12.45 mmol) in dry MeOH (20 mL) and the reaction mixture was stirred under an atmosphere of H₂ (g) at 1 atm and at ambient temperature until the reaction was complete. The reaction mixture was filtered and concentrated in vacuo. The residue was purified by flash chromatography on silica (0-99% of MTBE in hexane) to give the title compound (2.8 g, 77%); MS (ESI) m/z [M+H]⁺ 278.2.

### Intermediate 89

### Methyl 2-(chloromethyl)-1-((1-(cyanomethyl)cyclopropyl)methyl)-4-fluoro-1H-benzo[d]imidazole-6-carboxylate

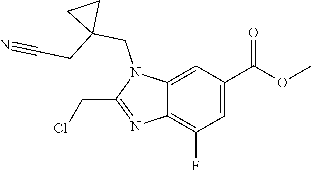 [SMILES: COC(=O)c1cc(F)c2nc(CCl)n(CC3(CC#N)CC3)c2c1]

2-Chloro-1,1,1-trimethoxyethane (1.34 mL, 9.92 mmol) and pTsOH (155 mg, 902 μmol) were added to a solution of methyl 4-amino-3-(((1-(cyanomethyl)cyclopropyl)methyl)amino)-5-fluorobenzoate Intermediate 88 (2.5 g, 9.02 mmol) in THF (50 mL) and the reaction mixture was stirred at 50° C. overnight. The reaction mixture was poured into water and extracted with EtOAc (3×10 mL). The combined organic layer was washed with brine and concentrated under vacuum to give the title compound (2.25 g, 67%); MS (ESI) m/z [M+H]⁺ 336.2.

### Intermediate 90

### Methyl 2-(chloromethyl)-1-((1-cyanocyclopropyl)methyl)-4-fluoro-1H-benzo[d]imidazole-6-carboxylate

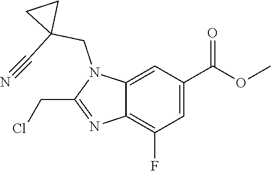 [SMILES: COC(=O)c1cc(F)c2nc(CCl)n(CC3(C#N)CC3)c2c1]

The title compound was prepared in three steps as described for Intermediate 89 from methyl 3,5-difluoro-4-nitrobenzoate and 1-(aminomethyl)cyclopropane-1-carbonitrile hydrochloride to give the title compound (2.35 g, 69%); MS (ESI) m/z [M+H]⁺ 322.0.

### Intermediate 91

### Methyl 3-chloro-5-(((1-cyanocyclopropyl)methyl)amino)-4-nitrobenzoate

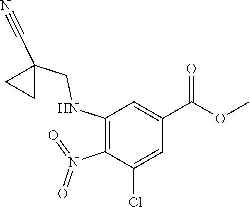 [SMILES: COC(=O)c1cc(Cl)c([N+](=O)[O-])c(NCC2(C#N)CC2)c1]

1-(Aminomethyl)cyclopropane-1-carbonitrile hydrochloride (1.76 g, 13.24 mmol) and DIPEA (5.13 g, 39.72 mmol) was slowly added to a colorless solution of methyl 3-chloro-5-fluoro-4-nitrobenzoate (3.09 g, 13.24 mmol) in DMF (70 mL) and the reaction mixture was stirred at 60° C. for 16 h. The reaction mixture was poured into water (250 mL) and extracted with DCM (3×80 mL). The combined organic layer was dried over Na₂SO₄, filtered, and concentrated under reduced pressure to give the crude title compound (3.6 g); MS (ESI) m/z [M+H]⁺ 310.0.

### Intermediate 92

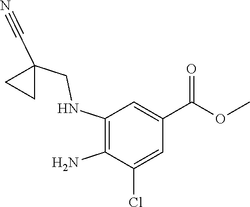 [SMILES: COC(=O)c1cc(Cl)c(N)c(NCC2(C#N)CC2)c1]

### Methyl 4-amino-3-chloro-5-(((1-cyanocyclopropyl)methyl)amino)benzoate

Wet Pt/C (10%, 0.12 g) was added to a yellow suspension of crude methyl 3-chloro-5-(((1-cyanocyclopropyl)methyl)amino)-4-nitrobenzoate Intermediate 91 (3.6 g) in MeOH (180 mL) and the reaction mixture was stirred under an atmosphere of H₂(g) at 1 atm and at 20° C. for 36 h. The reaction mixture was filtered and the filter cake was washed with MeOH (100 mL). The filtrate was concentrated under reduced pressure and the crude residue was purified by flash chromatography on silica (0-99% MTBE in hexane) to give the title compound (0.68 g, 21%); MS (ESI) m/z [M+H]⁺ 280.2.

### Intermediate 93

### Methyl 4-chloro-2-(chloromethyl)-1-((1-cyanocyclopropyl)methyl)-1H-benzo[d]imidazole-6-carboxylate

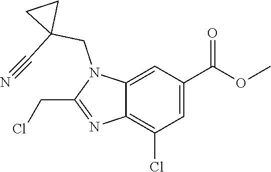 [SMILES: COC(=O)c1cc(Cl)c2nc(CCl)n(CC3(C#N)CC3)c2c1]

2-Chloro-1,1,1-trimethoxyethane (327 mg, 2.11 mmol) and pTsOH (33 mg, 192 μmol) were added to a stirred solution of methyl 4-amino-3-chloro-5-(((1-cyanocyclopropyl)methyl)amino)benzoate Intermediate 92 (538 mg, 1.92 mmol) in MeCN (50 mL) and the reaction mixture was heated at 60° C. for 2 h. The reaction mixture was cooled to rt and evaporated under reduced pressure. The residue was diluted with EtOAc (70 mL), and washed with NaHCO₃ (30 mL) and water (30 mL). The organic layer was dried over anhydrous Na₂SO₄ and concentrated to give the title compound (0.6 g, 87%); MS (ESI) m/z [M+H]⁺ 280.2.

### Intermediate 94

### Methyl 3-chloro-5-(((1-(cyanomethyl)cyclopropyl)methyl)amino)-4-nitrobenzoate

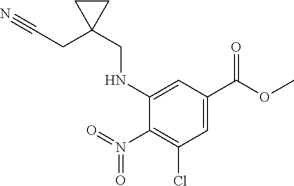 [SMILES: COC(=O)c1cc(Cl)c([N+](=O)[O-])c(NCC2(CC#N)CC2)c1]

A mixture of methyl 3-chloro-5-fluoro-4-nitrobenzoate (5.05 g, 21.62 mmol), 2-(1-(aminomethyl)cyclopropyl)acetonitrile hydrochloride (3.17 g, 21.62 mmol) and DIPEA (8.38 g, 64.87 mmol) in THF (200 mL) was heated at 50° C. for 14 h. The reaction mixture was evaporated under reduced pressure, and the residue was diluted with EtOAc (150 mL). The organic layer was washed with water (2×50 mL), dried over Na₂SO₄, filtered, and concentrated under reduced pressure to give the crude title compound (5.73 g); MS (ESI) m/z [M+H]⁺ 324.0.

### Intermediate 95

### Methyl 4-amino-3-chloro-5-(((1-(cyanomethyl)cyclopropyl)methyl)amino)benzoate

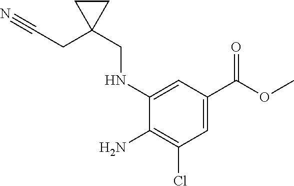 [SMILES: COC(=O)c1cc(Cl)c(N)c(NCC2(CC#N)CC2)c1]

Wet Pt/C (10%, 0.2 g) was added to a suspension of methyl 3-chloro-5-(((1-(cyanomethyl)cyclopropyl)methyl)amino)-4-nitrobenzoate Intermediate 94 (5.73 g, 17.7 mmol) in MeOH (200 mL) and the reaction mixture was stirred under an atmosphere of H₂ (g) at 1 atm and at 20° C. for 64 h. The reaction mixture was filtered, and the catalyst carefully washed with MeOH (100 mL). The filtrate was concentrated under reduced pressure. The crude compound was purified by flash chromatography on silica (0-99% MTBE in hexane) to give the title compound (3.7 g, 71%); ¹H NMR (500 MHz, CDCl₃) δ 7.66 (s, 1H), 7.34 (s, 1H), 3.87 (s, 3H), 3.18 (s, 2H), 2.61 (s, 2H), 1.58 (s, 3H), 0.76 (s, 4H).

### Intermediate 96

### Methyl 4-chloro-2-(chloromethyl)-1-((1-(cyanomethyl)cyclopropyl)methyl)-1H-benzo[d]imidazole-6-carboxylate

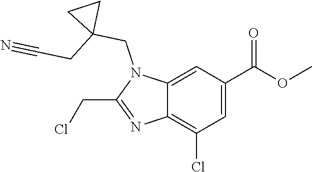 [SMILES: COC(=O)c1cc(Cl)c2nc(CCl)n(CC3(CC#N)CC3)c2c1]

2-Chloro-1,1,1-trimethoxyethane (435 mg, 2.81 mmol) and pTsOH (44 mg, 256 μmol) were added to a stirred solution of methyl 4-amino-3-chloro-5-(((1-(cyanomethyl)cyclopropyl)methyl)amino)benzoate Intermediate 95 (751 mg, 2.56 mmol) in MeCN (100 mL) and the reaction mixture was heated at 60° C. for 2 h. The reaction mixture was cooled to rt and then evaporated under reduced pressure. The residue was diluted with EtOAc (70 mL) and the mixture was washed with NaHCO₃ (30 mL) and water (30 mL). The organic layer was dried over Na₂SO₄ and evaporated at reduced pressure to give the crude title compound (0.72 g); MS (ESI) m/z [M+H]⁺ 352.0.

### Intermediate 97

### Methyl 3-(((1-(cyanomethyl)cyclopropyl)methyl)amino)-4-nitrobenzoate

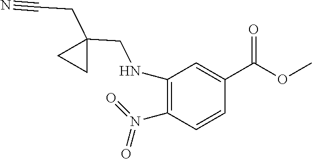 [SMILES: COC(=O)c1ccc([N+](=O)[O-])c(NCC2(CC#N)CC2)c1]

A mixture of methyl 3-fluoro-4-nitrobenzoate (3.44 g, 17.28 mmol), 2-[1-(aminomethyl)cyclopropyl]acetonitrile hydrochloride (2.53 g, 17.28 mmol) and DIPEA (6.7 g, 51.85 mmol) in MeCN (50 mL) was stirred at 50° C. for 12 h. The reaction mixture was cooled to rt, then diluted with water and extracted with DCM (3×100 mL). The combined organic layer was washed with brine (100 mL), dried over anhydrous Na₂SO₄, and filtered. The filtrate was concentrated at reduced pressure to give the title compound (5.0 g, 80%); MS (ESI) m/z [M+H]⁺ 290.0.

### Intermediate 98

### Methyl 4-amino-3-(((1-(cyanomethyl)cyclopropyl)methyl)amino)benzoate

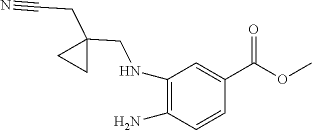 [SMILES: COC(=O)c1ccc(N)c(NCC2(CC#N)CC2)c1]

Methyl 3-(((1-(cyanomethyl)cyclopropyl)methyl)amino)-4-nitrobenzoate Intermediate 97 (5.02 g, 17.35 mmol) was dissolved in dry MeOH (50 mL) and treated with Pt/C (1% wt, 0.2 g, 5.21 mmol). The reaction mixture was hydrogenated under an atmosphere of H₂ (g) at 1 atm and at ambient temperature until the reaction was complete. The reaction mixture was filtered and the filtrate was concentrated in vacuo to give the title compound (4.5 g, 60%); MS (ESI) m/z [M+H]⁺ 260.2.

### Intermediate 99

### Methyl 2-(chloromethyl)-1-((1-(cyanomethyl)cyclopropyl)methyl)-1H-benzo[d]imidazole-6-carboxylate

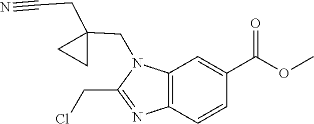 [SMILES: COC(=O)c1ccc2nc(CCl)n(CC3(CC#N)CC3)c2c1]

2-Chloro-1,1,1-trimethoxyethane (1.07 g, 6.92 mmol) and pTsOH (54.19 mg, 315 μmol) was added to a solution of methyl 4-amino-3-(((1-(cyanomethyl)cyclopropyl)methyl)amino)benzoate Intermediate 98 (1.63 g, 6.29 mmol) in MeCN (20 mL) and the reaction mixture was stirred at 60° C. overnight. The reaction mixture was poured into water and extracted with DCM (3×15 mL). The combined organic layer was washed with brine and concentrated in vacuo to give the title compound (1.8 g, 89%); MS (ESI) m/z [M+H]⁺ 318.0.

### Intermediate 100

### Methyl 2-(chloromethyl)-1-((1-cyanocyclopropyl)methyl)-1H-benzo[d]imidazole-6-carboxylate

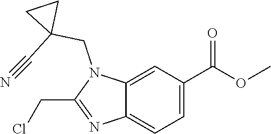 [SMILES: COC(=O)c1ccc2nc(CCl)n(CC3(C#N)CC3)c2c1]

The title compound was prepared in three steps from methyl 3-fluoro-4-nitrobenzoate and 1-(aminomethyl)cyclopropane-1-carbonitrile hydrochloride as described for Intermediate 99 to give the title compound (700 mg, 80%); MS (ESI) m/z [M+H]⁺ 304.2.

### Intermediate 101

### N-(2-(1H-pyrazol-1-yl)ethyl)-5-bromo-3-chloro-2-nitroaniline

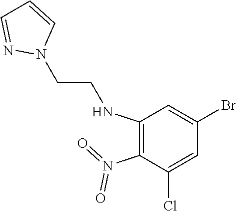 [SMILES: O=[N+]([O-])c1c(Cl)cc(Br)cc1NCCn1cccn1]

A mixture of 5-bromo-1-chloro-3-fluoro-2-nitrobenzene (2.03 g, 7.98 mmol), 2-(1H-pyrazol-1-yl)ethan-1-amine (0.89 g, 8.01 mmol), and DIPEA (2.79 mL, 15.96 mmol) in MeCN (5 mL) was stirred at 45° C. overnight. The reaction mixture was cooled to rt and concentrated at reduced pressure. The crude product was purified by flash chromatography on silica (0-50% EtOAC in heptane) to give the title compound (2.55 g, 92%) as a yellow powder; MS (ESI) m/z [M+H]⁺ 344.9.

### Intermediate 102

### N1-(2-(1H-pyrazol-1-yl)ethyl)-5-bromo-3-chlorobenzene-1,2-diamine

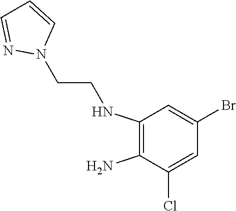 [SMILES: Nc1c(Cl)cc(Br)cc1NCCn1cccn1]

A solution of trichlorosilane (1.721 mL, 17.06 mmol) in MeCN (5 mL) was added dropwise over 15 min and under an atmosphere of N₂(g) to an ice-cold solution of N-(2-(1H-pyrazol-1-yl)ethyl)-5-bromo-3-chloro-2-nitroaniline Intermediate 101 (1.67 g, 4.83 mmol) and DIPEA (4.5 mL, 25.76 mmol) in MeCN (30 mL). The cooling bath was removed and the reaction mixture was stirred at rt overnight. Sat NaHCO₃ (aq, 30 mL) was added dropwise to the reaction mixture and it was then diluted with water and extracted with EtOAc (4×50 mL). The combined organic layer was dried over MgSO₄, filtered and concentrated and the crude residue was purified by preparative HPLC, PrepMethod H (gradient 30-70%) to give the title compound (0.750 g, 49%) as a dark oil; MS (ESI) m/z [M+H]⁺ 315.20.

### Intermediate 103

### Methyl 3-((2-(1H-pyrazol-1-yl)ethyl)amino)-4-amino-5-chlorobenzoate

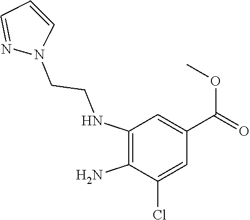 [SMILES: COC(=O)c1cc(Cl)c(N)c(NCCn2cccn2)c1]

Pd(dppf)Cl₂ (152 mg, 0.21 mmol) was added to a mixture of N1-(2-(1H-pyrazol-1-yl)ethyl)-5-bromo-3-chlorobenzene-1,2-diamine Intermediate 102 (0.75 g, 2.38 mmol) and DIPEA (4.15 mL, 23.76 mmol) in MeOH:DMSO (4:1, 5 mL) and the reaction mixture was stirred under an atmosphere of CO (g) (9 atm) at 85° C. for 16 h. The reaction mixture was filtered through a pad of celite and the pad was rinsed with MeOH until the washings were colourless. The filtrate was concentrated and the residue was purified by preparative HPLC, PrepMethod H (gradient:20-60%) to give the title compound (0.372 g, 53%) as a light-brown solid; MS (ESI) m/z [M+H]⁺ 295.1.

### Intermediate 104

### Methyl 1-(2-(1H-pyrazol-1-yl)ethyl)-4-chloro-2-(chloromethyl)-1H-benzo[d]imidazole-6-carboxylate

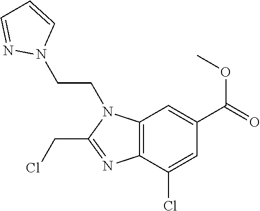 [SMILES: COC(=O)c1cc(Cl)c2nc(CCl)n(CCn3cccn3)c2c1]

2-Chloro-1,1,1-trimethoxyethane (0.247 mL, 1.84 mmol) was added to a solution of methyl 3-((2-(1H-pyrazol-1-yl)ethyl)amino)-4-amino-5-chlorobenzoate Intermediate 103 (0.361 g, 1.22 mmol) and pTsOH hydrate (0.025 g, 0.13 mmol) in MeCN (6 mL) and the reaction mixture was stirred at 50° C. for 2 h, then at rt for 2 days. 2-Chloro-1,1,1-trimethoxyethane (0.100 mL, 0.74 mmol) was added and the reaction mixture was heated at 50° C. for 2 h. EtOAc (50 mL) was added to the reaction mixture and the mixture was washed with sat NaHCO₃ (aq, 2×30 mL). The organic layer was dried over MgSO₄, filtered, and concentrated at reduced pressure to give the title compound (0.402 g, 93%) as a light-brown solid; MS (ESI) m/z [M+H]⁺ 353.2.

### Intermediate 105

### rac-Methyl 1-(2-(1H-pyrazol-1-yl)ethyl)-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylate

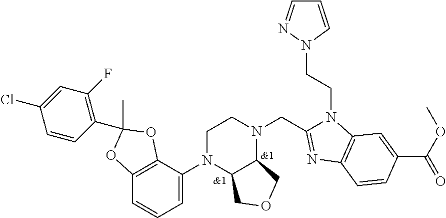 [SMILES: COC(=O)c1ccc2nc(CN3CCN(c4cccc5c4OC(C)(c4ccc(Cl)cc4F)O5)[C@@H]4COC[C@@H]43)n(CCn3cccn3)c2c1]

A mixture of rac-(4aR,7aS)-1-(2-(5-chloropyridin-2-yl)-2-methylbenzo[d][1,3]dioxol-4-yl)octahydrofuro[3,4-b]pyrazine dihydrochloride Intermediate 36 Step a) (0.625 g, 1.26 mmol), methyl 1-(2-(1H-pyrazol-1-yl)ethyl)-2-(chloromethyl)-1H-benzo[d]imidazole-6-carboxylate Intermediate 60 (0.407 g, 1.28 mmol), and K₂CO₃ (0.76 g, 5.50 mmol) in MeCN:H₂O (20:1, 10.5 mL) was stirred at 70° C. for 24 h. The reaction mixture was concentrated and the residue was dissolved in EtOAc (100 mL). The organic layer was washed with water (2×50 mL), dried over MgSO₄, and concentrated. The crude product was purified by flash chromatography on silica (EtOAc) to give the title compound (0.665 g, 80%) as a light-brown oil; MS (ESI) m/z [M+H]⁺ 656.4.

### Intermediate 106

### rac-Methyl 1-(2-(1H-pyrazol-1-yl)ethyl)-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylate

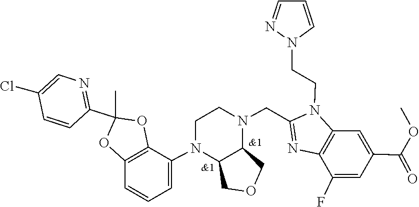 [SMILES: COC(=O)c1cc(F)c2nc(CN3CCN(c4cccc5c4OC(C)(c4ccc(Cl)cn4)O5)[C@@H]4COC[C@@H]43)n(CCn3cccn3)c2c1]

A mixture of rac-(4aR,7aS)-1-(2-(5-chloropyridin-2-yl)-2-methylbenzo[d][1,3]dioxol-4-yl)octahydrofuro[3,4-b]pyrazine dihydrochloride Intermediate 36 Step a) (0.402 g, 0.81 mmol), methyl 1-(2-(1H-pyrazol-1-yl)ethyl)-2-(chloromethyl)-4-fluoro-1H-benzo[d]imidazole-6-carboxylate Intermediate 64 (0.275 g, 0.82 mmol), and K₂CO₃ (0.552 g, 3.99 mmol) in a MeCN:H₂O (20:1, 6.3 mL) was stirred at 70° C. for 2 days. The reaction mixture was concentrated, and the residue was dissolved in EtOAc (100 mL). The organic layer was washed with water (2×50 mL), dried over MgSO₄, and concentrated. The crude product was purified by flash chromatography on silica (EtOAc) to give the title compound (0.425 g, 78%) as a solid foam; MS (ESI) m/z [M+H]⁺ 674.62.

### Intermediate 107

### Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylate

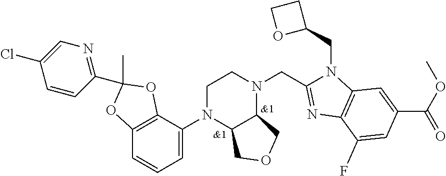 [SMILES: COC(=O)c1cc(F)c2nc(CN3CCN(c4cccc5c4OC(C)(c4ccc(Cl)cn4)O5)[C@@H]4COC[C@@H]43)n(C[C@@H]3CCO3)c2c1]

### Step a) rac-(4aR,7aS)-1-(2-(5-Chloropyridin-2-yl)-2-methylbenzo[d][1,3]dioxol-4-yl)octahydrofuro[3,4-b]pyrazine

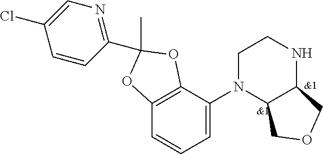 [SMILES: CC1(c2ccc(Cl)cn2)Oc2cccc(N3CCN[C@H]4COC[C@H]43)c2O1]

TFA (279 μL, 3.62 mmol) was added to a solution of rac-tert-butyl (4aR,7aS)-4-(2-(5-chloropyridin-2-yl)-2-methylbenzo[d][1,3]dioxol-4-yl)hexahydrofuro[3,4-b]pyrazine-1(2H)-carboxylate Intermediate 20 (343 mg, 0.72 mmol) in DCM (3.0 mL) and the reaction mixture was stirred at rt for 30 min. The reaction mixture was evaporated at reduced pressure and co-evaporated with toluene to give the TFA salt of the sub-title compound; MS (ESI) m/z [M+H]⁺ 374.3. The crude title compound was used in the next step.

### Step b) Methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylate

rac-(4aR,7aS)-1-(2-(5-Chloropyridin-2-yl)-2-methylbenzo[d][1,3]dioxol-4-yl)octahydrofuro[3,4-b]pyrazine trifluoroacetate Intermediate 107 Step a) was dissolved in MeCN (3 mL) and K₂CO₃ (300 mg, 2.17 mmol) and methyl (S)-2-(chloromethyl)-4-fluoro-1-((tetrahydrofuran-2-yl)methyl)-1H-benzo[d]imidazole-6-carboxylate Intermediate 65 (284 mg, 0.87 mmol) were added and the reaction mixture was heated at 60° C. for 18 h, then at 70° C. for 24 h. The reaction mixture was cooled to rt and filtered through Celite. The filtrate was collected, evaporated at reduced pressure and the crude residue was purified by flash chromatography on silica (EtOAc:heptane, 30:70, then 30-100% EtOAc in heptane) to give the title compound (0.443 g, 92% in two steps); MS (ESI) m/z [M+H]⁺ 664.4.

### Intermediate 108

### rac-Methyl 2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylate

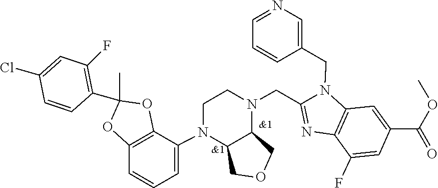 [SMILES: COC(=O)c1cc(F)c2nc(CN3CCN(c4cccc5c4OC(C)(c4ccc(Cl)cc4F)O5)[C@@H]4COC[C@@H]43)n(Cc3cccnc3)c2c1]

Methyl 2-(chloromethyl)-4-fluoro-1-(pyridin-3-ylmethyl)-1H-benzo[d]imidazole-6-carboxylate dihydrochloride Intermediate 68 (429 mg, 1.05 mmol) was added to a suspension of rac-(4aR,7aS)-1-(2-(4-chloro-2-fluorophenyl)-2-methylbenzo[d][1,3]dioxol-4-yl)octahydrofuro[3,4-b]pyrazine hydrochloride Intermediate 22 (412 mg, 1.05 mmol), DIPEA (817 mg, 6.32 mmol) and NaI (632 mg, 4.22 mmol) in DMF (25 mL) and the reaction mixture was stirred at 60° C. for 18 h. The reaction mixture was concentrated in vacuo, then diluted with water (20 mL) and extracted with DCM. The combined organic layer was dried over Na₂SO₄, filtered, and concentrated in vacuo. The crude residue was purified by preparative HPLC, PrepMethod D (gradient:10-50%) to give the title compound (83 mg, 11%); MS (ESI) m/z [M+H]⁺ 688.2.

### Intermediate 109

### Methyl 4-chloro-2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate

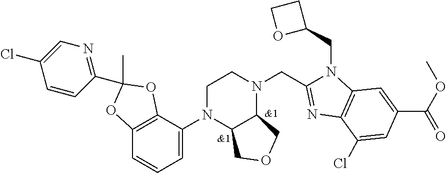 [SMILES: COC(=O)c1cc(Cl)c2nc(CN3CCN(c4cccc5c4OC(C)(c4ccc(Cl)cn4)O5)[C@@H]4COC[C@@H]43)n(C[C@@H]3CCO3)c2c1]

DIPEA (620 mg, 4.8 mmol) was added to a suspension of rac-(4aR,7aS)-1-(2-(5-chloropyridin-2-yl)-2-methylbenzo[d][1,3]dioxol-4-yl)octahydrofuro[3,4-b]pyrazine hydrochloride Intermediate 36 Step a) (449 mg, 1.2 mmol), methyl (S)-4-chloro-2-(chloromethyl)-1-(oxetan-2-ylmethyl)-1H-benzo[d]imidazole-6-carboxylate Intermediate 72 (395 mg, 1.2 mmol) and NaI (540 mg, 3.6 mmol) in MeCN (5 mL) at 20° C., and the reaction mixture was heated at 60° C. overnight. The reaction mixture was concentrated under reduced pressure and the residue was suspended in EtOAc (10 mL). The organic layer was washed with sat NaHCO₃ (aq, 20 mL), brine (20 mL), dried over Na₂SO₄, and concentrated under reduced pressure. The residue was purified by preparative HPLC, PrepMethod D (gradient 40-50%) to give the title compound (123 mg, 15%); MS (ESI) m/z [M+H]⁺ 666.2.

### Intermediate 110

### Methyl 4-chloro-2-(((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate

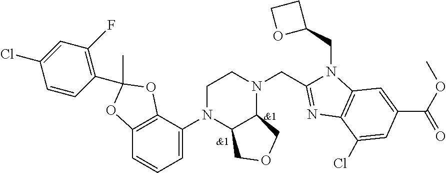 [SMILES: COC(=O)c1cc(Cl)c2nc(CN3CCN(c4cccc5c4OC(C)(c4ccc(Cl)cc4F)O5)[C@@H]4COC[C@@H]43)n(C[C@@H]3CCO3)c2c1]

Cs₂CO₃ (1.5 g, 4.61 mmol) was added to a suspension of rac-(4aR,7aS)-1-(2-(4-chloro-2-fluorophenyl)-2-methylbenzo[d][1,3]dioxol-4-yl)octahydrofuro[3,4-b]pyrazine hydrochloride Intermediate 22 (515 mg, 1.32 mmol), methyl (S)-4-chloro-2-(chloromethyl)-1-(oxetan-2-ylmethyl)-1H-benzo[d]imidazole-6-carboxylate Intermediate 72 (433 mg, 1.32 mmol) and NaI (395 mg, 2.63 mmol) in MeCN (5 mL) at 20° C. and the reaction mixture was heated at 60° C. overnight. The reaction mixture was concentrated under reduced pressure and the residue was suspended in EtOAc (10 mL). The organic layer was washed with sat NaHCO₃ (aq, 20 mL), brine (20 mL), dried over Na₂SO₄, and concentrated under reduced pressure. The residue was purified by preparative HPLC, PrepMethod D (gradient: 40-65%) to give the title compound (158 mg, 17%); MS (ESI) m/z [M+H]⁺ 683.3.

### Intermediate 111

### Methyl 2-(((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate

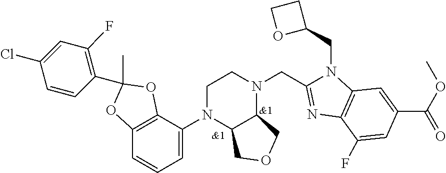 [SMILES: COC(=O)c1cc(F)c2nc(CN3CCN(c4cccc5c4OC(C)(c4ccc(Cl)cc4F)O5)[C@@H]4COC[C@@H]43)n(C[C@@H]3CCO3)c2c1]

Methyl (S)-2-(chloromethyl)-4-fluoro-1-(oxetan-2-ylmethyl)-1H-benzo[d]imidazole-6-carboxylate Intermediate 19 (375 mg, 1.20 mmol) was added to a suspension of rac-(4aR,7aS)-1-(2-(4-chloro-2-fluorophenyl)-2-methylbenzo[d][1,3]dioxol-4-yl)octahydrofuro[3,4-b]pyrazine hydrochloride Intermediate 22 (469 mg, 1.2 mmol), Cs₂CO₃ (1.36 g, 4.2 mmol) and NaI (360 mg, 2.4 mmol) in DMF (6 mL), and the reaction mixture was stirred at 60° C. for 18 h. The reaction mixture was concentrated in vacuo, the residue was diluted with water (20 mL) and the obtained mixture was extracted with EtOAc (3×30 mL). The combined organic layer was dried over Na₂SO₄, filtered, and concentrated in vacuo. The residue was purified by preparative HPLC, PrepMethod D (gradient: 0-50%) to give the title compound (114 mg, 17%); MS (ESI) m/z [M+H]⁺ 667.2.

### Intermediate 112

### rac-Methyl 2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylate

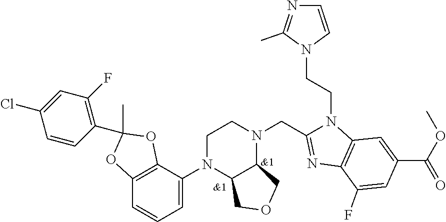 [SMILES: COC(=O)c1cc(F)c2nc(CN3CCN(c4cccc5c4OC(C)(c4ccc(Cl)cc4F)O5)[C@@H]4COC[C@@H]43)n(CCn3ccnc3C)c2c1]

Methyl 2-(chloromethyl)-4-fluoro-1-(2-(2-methyl-1H-imidazol-1-yl)ethyl)-1H-benzo[d]imidazole-6-carboxylate Intermediate 76 (300 mg, 709 μmol) was added to a suspension of rac-(4aR,7aS)-1-(2-(4-chloro-2-fluorophenyl)-2-methylbenzo[d][1,3]dioxol-4-yl)octahydrofuro[3,4-b]pyrazine hydrochloride Intermediate 22 (277 mg, 709 μmol), DIPEA (550 mg, 4.25 mmol), and NaI (425 mg, 2.84 mmol) in DMF (20 mL) and the reaction mixture was stirred at 60° C. for 18 h. The reaction mixture was concentrated in vacuo, diluted with water (20 mL) and extracted with DCM (3×8 mL). The combined organic layer was dried over Na₂SO₄, filtered, and concentrated in vacuo. The residue was purified by preparative HPLC, PrepMethod D (gradient: 30-80%) to give the title compound (136 mg, 27%); MS (ESI) m/z [M+H]⁺ 705.0.

### Intermediate 113

### rac-Methyl 4-chloro-2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylate

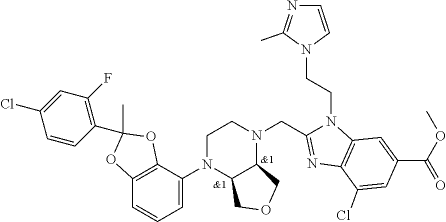 [SMILES: COC(=O)c1cc(Cl)c2nc(CN3CCN(c4cccc5c4OC(C)(c4ccc(Cl)cc4F)O5)[C@@H]4COC[C@@H]43)n(CCn3ccnc3C)c2c1]

Methyl 4-chloro-2-(chloromethyl)-1-(2-(2-methyl-1H-imidazol-1-yl)ethyl)-1H-benzo[d]imidazole-6-carboxylate dihydrochloride Intermediate 80 (263 mg, 717 μmol.) was added to a solution of rac-(4aR,7aS)-1-(2-(4-chloro-2-fluorophenyl)-2-methylbenzo[d][1,3]dioxol-4-yl)octahydrofuro[3,4-b]pyrazine hydrochloride Intermediate 22 (280 mg, 717 μmol), DIPEA (556 mg, 4.3 mmol), and NaI (430 mg, 2.87 mmol) in DMF (20 mL) and the reaction mixture was stirred at 60° C. for 18 h. The reaction mixture was concentrated in vacuo, diluted with water (20 mL), and extracted with DCM (3×10 mL). The combined organic layer was washed with brine and water, dried over Na₂SO₄, filtered, and concentrated in vacuo. The residue was purified by preparative HPLC, PrepMethod D (gradient 10-50%) to give the title compound (76 mg, 15%); MS (ESI) m/z [M+H]⁺ 721.2.

### Intermediate 114

### rac-Methyl 4-chloro-2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylate

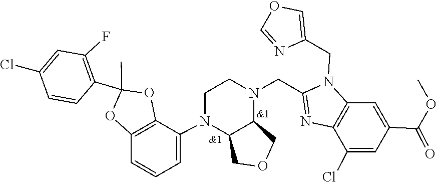 [SMILES: COC(=O)c1cc(Cl)c2nc(CN3CCN(c4cccc5c4OC(C)(c4ccc(Cl)cc4F)O5)[C@@H]4COC[C@@H]43)n(Cc3cocn3)c2c1]

Methyl 4-chloro-2-(chloromethyl)-1-(oxazol-4-ylmethyl)-1H-benzo[d]imidazole-6-carboxylate Intermediate 83 (248 mg, 759 μmol) was added to a suspension rac-(4aR,7aS)-1-(2-(4-chloro-2-fluorophenyl)-2-methylbenzo[d][1,3]dioxol-4-yl)octahydrofuro[3,4-b]pyrazine hydrochloride Intermediate 22 (297 mg, 759 μmol), DIPEA (343 mg, 2.66 mmol), and NaI (455 mg, 3.04 mmol) in DMF (20 mL) and the reaction mixture was stirred at 60° C. for 18 h. The reaction mixture was concentrated in vacuo, then diluted with water (20 mL) and extracted with DCM (3×20 mL). The combined organic layer was dried over Na₂SO₄, filtered, and concentrated in vacuo. The residue was purified by preparative HPLC, PrepMethod D (gradient: 10-50%) to give the title compound (316 mg, 56%); MS (ESI) m/z [M+H]⁺ 694.0.

### Intermediate 115

### rac-Methyl 2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylate

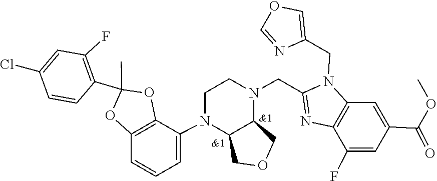 [SMILES: COC(=O)c1cc(F)c2nc(CN3CCN(c4cccc5c4OC(C)(c4ccc(Cl)cc4F)O5)[C@@H]4COC[C@@H]43)n(Cc3cocn3)c2c1]

Methyl 2-(chloromethyl)-4-fluoro-1-(oxazol-4-ylmethyl)-1H-benzo[d]imidazole-6-carboxylate Intermediate 86 (252 mg, 779 μmol) was added to a suspension of rac-(4aR,7aS)-1-(2-(4-chloro-2-fluorophenyl)-2-methylbenzo[d][1,3]dioxol-4-yl)octahydrofuro[3,4-b]pyrazine hydrochloride Intermediate 22 (304 mg, 779 μmol), DIPEA (352 mg, 2.73 mmol) and NaI (467 mg, 3.12 mmol) in DMF (20 mL) and the reaction mixture was stirred at 60° C. for 18 h. The reaction mixture was concentrated in vacuo, then diluted with water (20 mL) and extracted with DCM (3×10 mL). The combined organic layer was dried over Na₂SO₄, filtered, and concentrated in vacuo. The residue was purified by preparative HPLC, PrepMethod D (gradient: 10-50%) to give the title compound (108 mg, 20%); MS (ESI) m/z [M+H]⁺ 678.2.

### Intermediate 116

### rac-Methyl 2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylate

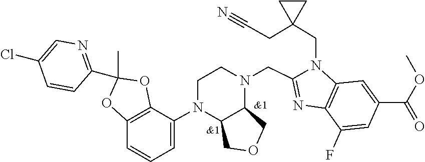 [SMILES: COC(=O)c1cc(F)c2nc(CN3CCN(c4cccc5c4OC(C)(c4ccc(Cl)cn4)O5)[C@@H]4COC[C@@H]43)n(CC3(CC#N)CC3)c2c1]

Methyl 2-(chloromethyl)-1-((1-(cyanomethyl)cyclopropyl)methyl)-4-fluoro-1H-benzo[d]imidazole-6-carboxylate Intermediate 89 (0.246 g, 0.73 mmol) was added to a suspension of rac-(4aR,7aS)-1-(2-(5-chloropyridin-2-yl)-2-methylbenzo[d][1,3]dioxol-4-yl)octahydrofuro[3,4-b]pyrazine hydrochloride Intermediate 36 Step a) (0.3 g, 0.73 mmol) and DIPEA (0.473 g, 4.10 mmol) in DMF (5 mL) and the reaction mixture was stirred at 50° C. for 16 h. The reaction mixture was concentrated in vacuo, then diluted with water (10 mL) and extracted with EtOAc (3×5 mL). The combined organic layer was dried over Na₂SO₄, filtered, and concentrated in vacuo. The residue was purified by preparative HPLC, PrepMethod D (gradient: 10-50%) to give the title compound (252 mg, 50%); MS (ESI) m/z [M+H]⁺ 673.0.

### Intermediate 117

### rac-Methyl 2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylate

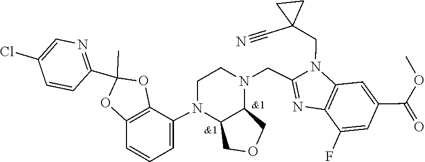 [SMILES: COC(=O)c1cc(F)c2nc(CN3CCN(c4cccc5c4OC(C)(c4ccc(Cl)cn4)O5)[C@@H]4COC[C@@H]43)n(CC3(C#N)CC3)c2c1]

rac-(4aR,7aS)-1-(2-(5-Chloropyridin-2-yl)-2-methylbenzo[d][1,3]dioxol-4-yl)octahydrofuro[3,4-b]pyrazine hydrochloride Intermediate 36 Step a) (0.3 g, 0.8 mmol) was added to a suspension of methyl 2-(chloromethyl)-1-((1-cyanocyclopropyl)methyl)-4-fluoro-1H-benzo[d]imidazole-6-carboxylate Intermediate 90 (0.258 g, 0.8 mmol) and DIPEA (0.519 g, 4 mmol) in MeCN (10 mL) and the reaction mixture was stirred at 60° C. for 18 h. The reaction mixture was concentrated in vacuo, diluted with water (70 mL) and extracted with DCM (2×150 mL). The combined organic layer was dried over Na₂SO₄, filtered, and concentrated in vacuo. The residue was purified by preparative HPLC, PrepMethod D (gradient: 10-50%) to give the title compound (220 mg, 43%); MS (ESI) m/z [M+H]⁺ 659.0.

### Intermediate 118

### rac-Methyl 4-chloro-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate

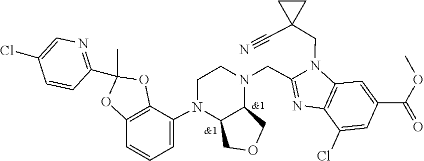 [SMILES: COC(=O)c1cc(Cl)c2nc(CN3CCN(c4cccc5c4OC(C)(c4ccc(Cl)cn4)O5)[C@@H]4COC[C@@H]43)n(CC3(C#N)CC3)c2c1]

rac-(4aR,7aS)-1-(2-(5-Chloropyridin-2-yl)-2-methylbenzo[d][1,3]dioxol-4-yl)octahydrofuro[3,4-b]pyrazine hydrochloride Intermediate 36 Step a) (476 mg, 1.27 mmol) was added to a suspension of methyl 4-chloro-2-(chloromethyl)-1-((1-cyanocyclopropyl)methyl)-1H-benzo[d]imidazole-6-carboxylate Intermediate 93 (431 mg, 1.2 mmol), DIPEA (988 mg, 7.64 mmol) and NaI (764 mg, 5.1 mmol) in MeCN (150 mL) and the reaction mixture was stirred at 60° C. for 18 h. The reaction mixture was concentrated in vacuo, the residue was diluted with water (50 mL) and the mixture was extracted with DCM (2×85 mL). The combined organic layer was dried over Na₂SO₄, filtered, and concentrated in vacuo. The residue was purified by preparative HPLC, PrepMethod D, (gradient: 40-95%) to give the title compound (84 mg, 10%); MS (ESI) m/z [M+H]⁺ 677.2.

### Intermediate 119

### rac-Methyl 4-chloro-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylate

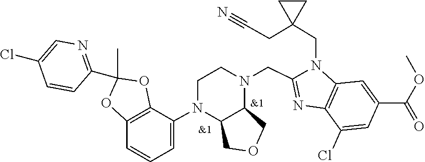 [SMILES: COC(=O)c1cc(Cl)c2nc(CN3CCN(c4cccc5c4OC(C)(c4ccc(Cl)cn4)O5)[C@@H]4COC[C@@H]43)n(CC3(CC#N)CC3)c2c1]

rac-(4aR,7aS)-1-(2-(5-Chloropyridin-2-yl)-2-methylbenzo[d][1,3]dioxol-4-yl)octahydrofuro[3,4-b]pyrazine hydrochloride Intermediate 36 Step a) (792 mg, 2.12 mmol) was added to a suspension of crude methyl 4-chloro-2-(chloromethyl)-1-((1-(cyanomethyl)cyclopropyl)methyl)-1H-benzo[d]imidazole-6-carboxylate Intermediate 96 (746 mg), DIPEA (1.64 g, 12.71 mmol) and NaI (1.27 g, 8.47 mmol) in MeCN (250 mL) and the reaction mixture was stirred at 60° C. for 18 h. The reaction mixture was concentrated in vacuo, diluted with water (70 mL) and extracted with DCM (2×150 mL). The combined organic layer was dried over Na₂SO₄, filtered, and concentrated in vacuo. The residue was purified by preparative HPLC, PrepMethod D (gradient 20-45%) to give the title compound (76 mg, 4%); MS (ESI) m/z [M+H]⁺ 689.2.

### Intermediate 120

### rac-Methyl 2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylate

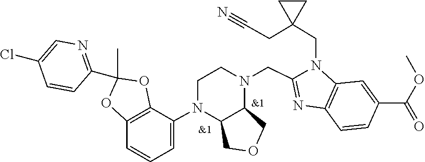 [SMILES: COC(=O)c1ccc2nc(CN3CCN(c4cccc5c4OC(C)(c4ccc(Cl)cn4)O5)[C@@H]4COC[C@@H]43)n(CC3(CC#N)CC3)c2c1]

Methyl 2-(chloromethyl)-1-((1-(cyanomethyl)cyclopropyl)methyl)-1H-benzo[d]imidazole-6-carboxylate Intermediate 99 (503 mg, 1.58 mmol) was added to a suspension of rac-(4aR,7aS)-1-(2-(5-chloropyridin-2-yl)-2-methylbenzo[d][1,3]dioxol-4-yl)octahydrofuro[3,4-b]pyrazine hydrochloride Intermediate 36 Step a) (650 mg, 1.58 mmol), DIPEA (819 mg, 6.34 mmol) and NaI (1.19 g, 7.92 mmol) in MeCN (10 mL) and the reaction mixture was stirred at 60° C. for 12 h. The reaction mixture was concentrated in vacuo, then diluted with water (10 mL) and extracted with DCM (3×15 mL). The combined organic layer was dried over Na₂SO₄, filtered, and concentrated in vacuo. The residue was purified by preparative HPLC, PrepMethod D (gradient: 10-50%) to give the title compound (500 mg, 50%); MS (ESI) m/z [M+H]⁺ 655.2.

### Intermediate 121

### rac-Methyl 2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate

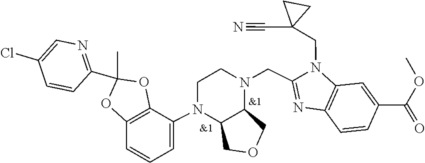 [SMILES: COC(=O)c1ccc2nc(CN3CCN(c4cccc5c4OC(C)(c4ccc(Cl)cn4)O5)[C@@H]4COC[C@@H]43)n(CC3(C#N)CC3)c2c1]

A mixture of methyl 2-(chloromethyl)-1-((1-cyanocyclopropyl)methyl)-1H-benzo[d]imidazole-6-carboxylate Intermediate 100 (492 mg, 1.62 mmol), rac-(4aR,7aS)-1-(2-(5-chloropyridin-2-yl)-2-methylbenzo[d][1,3]dioxol-4-yl)octahydrofuro[3,4-b]pyrazine hydrochloride Intermediate 36 Step a) (665 mg, 1.62 mmol), DIPEA (838 mg, 6.48 mmol) and NaI (1.21 g, 8.1 mmol) in MeCN (10 mL) was stirred at 60° C. for 12 h. The reaction mixture was concentrated in vacuo, then diluted with water (5 mL) and extracted with DCM (3×10 mL). The combined organic layer was dried over Na₂SO₄, filtered, and concentrated in vacuo. The residue was purified by preparative HPLC, PrepMethod D (gradient. 40-50%) to give the title compound (400 mg, 40%); MS (ESI) m/z [M+H]⁺ 641.0.

### Intermediate 122

### rac-Methyl 1-(2-(1H-pyrazol-1-yl)ethyl)-4-chloro-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylate

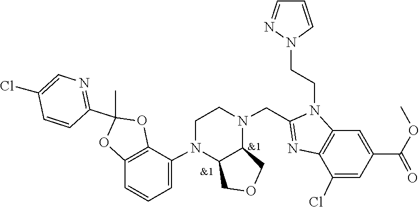 [SMILES: COC(=O)c1cc(Cl)c2nc(CN3CCN(c4cccc5c4OC(C)(c4ccc(Cl)cn4)O5)[C@@H]4COC[C@@H]43)n(CCn3cccn3)c2c1]

A mixture of rac-(4aR,7aS)-1-(2-(5-chloropyridin-2-yl)-2-methylbenzo[d][1,3]dioxol-4-yl)octahydrofuro[3,4-b]pyrazine dihydrochloride Intermediate 36 Step a) (0.55 g, 1.11 mmol), methyl 1-(2-(1H-pyrazol-1-yl)ethyl)-4-chloro-2-(chloromethyl)-1H-benzo[d]imidazole-6-carboxylate Intermediate 104 (0.39 g, 1.10 mmol), and K₂CO₃ (0.836 g, 6.05 mmol) in MeCN:H₂O (20:1, 10.5 mL) was stirred at 70° C. overnight and then concentrated at reduced pressure. The residue was dissolved in EtOAc (100 mL), and the organic layer was washed with water (2×50 mL), dried over MgSO₄, and concentrated. The crude product was purified by flash chromatography on silica (EtOAc) to give the title compound (0.642 g, 84%) as a solid off-white foam; MS (ESI) m/z [M+H]⁺ 690.4.

### Intermediate 123

### 2-(((4aS,7aR)-4-(2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid

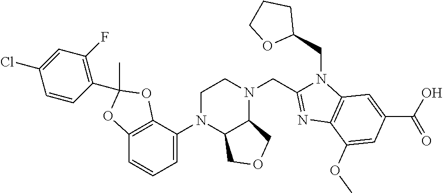 [SMILES: COc1cc(C(=O)O)cc2c1nc(CN1CCN(c3cccc4c3OC(C)(c3ccc(Cl)cc3F)O4)[C@@H]3COC[C@@H]31)n2C[C@@H]1CCCO1]

K₂CO₃ (1.204 g, 8.72 mmol) was added to (4aR,7aS)-1-(2-(4-chloro-2-fluorophenyl)-2-methylbenzo[d][1,3]dioxol-4-yl)octahydrofuro[3,4-b]pyrazine trifluoro acetate Intermediate 59 (880 mg, 1.74 mmol) and methyl (S)-2-(chloromethyl)-4-methoxy-1-((tetrahydrofuran-2-yl)methyl)-1H-benzo[d]imidazole-6-carboxylate Intermediate 7 (600 mg, 1.77 mmol) in MeCN (30 mL) and the reaction mixture was stirred at 25° C. for 8 h. The reaction mixture was filtered through Celite and the filter cake was washed with MeCN (3×50 mL). The filtrate was collected and concentrated at reduced pressure. The crude product was purified by flash chromatography on a C18 column (0-100% MeCN in water) to give the title compound (1.02 g, 85%) as a white solid; MS (ESI) m/z [M+H]⁺ 679.

### Intermediate 124

### rel-Methyl 1-(2-(1H-pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylate, Isomer 1

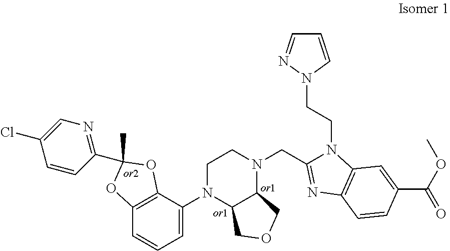 [SMILES: COC(=O)c1ccc2nc(CN3CCN(c4cccc5c4O[C@@](C)(c4ccc(Cl)cn4)O5)[C@@H]4COC[C@@H]43)n(CCn3cccn3)c2c1]

The stereoisomers of rac-methyl 1-(2-(1H-pyrazol-1-yl)ethyl)-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylate Intermediate 105 (828 mg, 1.26 mmol) were separated by chiral chromatography on a Lux C4 column (250×30 mm, 5 μm), eluted with 40% (EtOH, 20 mM DEA) in CO₂, 120 bar, at a flow rate of 120 mL/min, and detected at 266 nm;
the first eluted compound mixture was collected and evaporated to give a mixture of isomers that were separated by chiral chromatography on a YMC SJ (imob OJ) column (250×30 mm, 5 μm), eluted with 15% (EtOH, 20 mM DEA) in CO₂, 125 bar, at a flow rate of 135 mL/min and detected at 220 nm;
the first eluted compound was collected and evaporated to give the title compound, Isomer 1, Intermediate 124 (131 mg, 16%); MS (ESI) m/z [M+H]⁺ 656.4.

### Intermediate 125

### rel-Methyl 1-(2-(1H-pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylate, Isomer 1

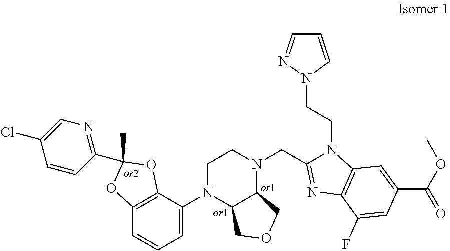 [SMILES: COC(=O)c1cc(F)c2nc(CN3CCN(c4cccc5c4O[C@@](C)(c4ccc(Cl)cn4)O5)[C@@H]4COC[C@@H]43)n(CCn3cccn3)c2c1]

The stereoisomers of rac-methyl 1-(2-(1H-pyrazol-1-yl)ethyl)-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylate Intermediate 106 (425 mg, 0.63 mmol) were separated by chiral chromatography on a Chiralpak-IBN column (250×30 mm, 5 μm), eluted with 30% (EtOH, 20 mM DEA) in CO₂, 130 bar, at a flow rate of 125 mL/min, and detected at 220 nm;
the first eluted compound mixture was collected and evaporated to give a mixture of isomers that were separated by chiral chromatography on a YMC SA (IA) column (250×30 mm, 5 μm), eluted with 15% (EtOH 20 mM DEA) in CO₂, 130 bar, at a flow rate of 120 mL/min and detected at 230 nm;
the first eluted compound was collected and evaporated to give the title compound, Isomer 1, Intermediate 125 (92 mg, 22%).

### Intermediate 126

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylate, Isomer 3

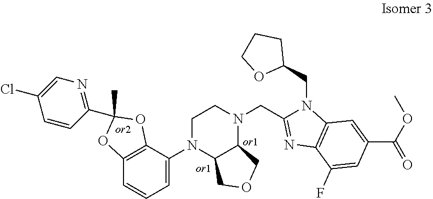 [SMILES: COC(=O)c1cc(F)c2nc(CN3CCN(c4cccc5c4O[C@@](C)(c4ccc(Cl)cn4)O5)[C@@H]4COC[C@@H]43)n(C[C@@H]3CCCO3)c2c1]

### Intermediate 127

### Methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylate, Isomer 4

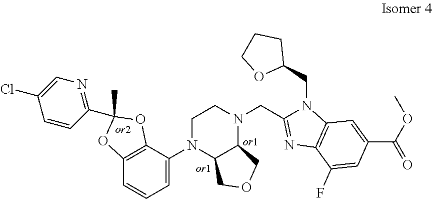 [SMILES: COC(=O)c1cc(F)c2nc(CN3CCN(c4cccc5c4O[C@@](C)(c4ccc(Cl)cn4)O5)[C@@H]4COC[C@@H]43)n(C[C@@H]3CCCO3)c2c1]

The stereoisomers of methyl 2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylate Intermediate 107 (495 mg, 0.75 mmol) were separated by chiral chromatography on a YMC SA (IA) column (250×30 mm, 5 μm), eluted with 15% (EtOH, 20 mM DEA) in CO₂, 125 bar, at a flow rate of 140 mL/min, and detected at 220 nm;
the second eluted compound mixture was collected and evaporated to give a mixture of isomers that were separated by chiral chromatography on a YMC SJ (imob OJ) column (250×30 mm, 5 μm), eluted with 17-22% (MeOH, 20 mM NH₃) in CO₂, 120 bar, at a flow rate of 100 mL/min and detected at 220 nm;
the first eluted compound was collected and evaporated to give the title compound, Isomer 3, Intermediate 126 (81 mg, 16%); MS (ESI) m/z [M+H]⁺ 664.6; and
the second eluted compound was collected and evaporate to give the title compound, Isomer 4, Intermediate 127 (83 mg, 17%); MS (ESI) m/z [M+H]⁺ 664.4.

### Intermediate 128

### rel-Methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylate, Isomer 1

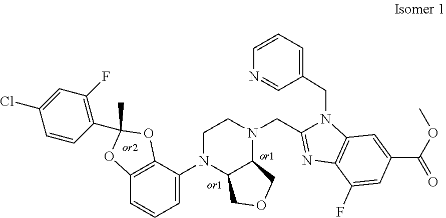 [SMILES: COC(=O)c1cc(F)c2nc(CN3CCN(c4cccc5c4O[C@@](C)(c4ccc(Cl)cc4F)O5)[C@@H]4COC[C@@H]43)n(Cc3cccnc3)c2c1]

### Intermediate 129

### rel-Methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylate, Isomer 2

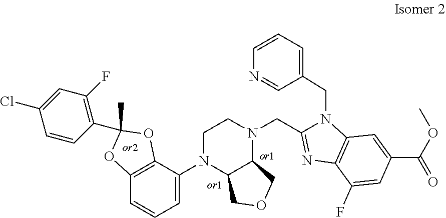 [SMILES: COC(=O)c1cc(F)c2nc(CN3CCN(c4cccc5c4O[C@@](C)(c4ccc(Cl)cc4F)O5)[C@@H]4COC[C@@H]43)n(Cc3cccnc3)c2c1]

### Intermediate 130

### rel-Methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylate, Isomer 4

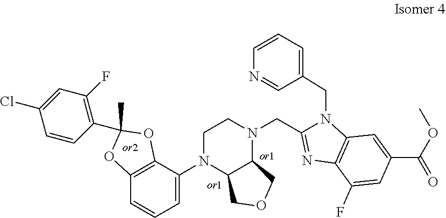 [SMILES: COC(=O)c1cc(F)c2nc(CN3CCN(c4cccc5c4O[C@@](C)(c4ccc(Cl)cc4F)O5)[C@@H]4COC[C@@H]43)n(Cc3cccnc3)c2c1]

The stereoisomers of rac-methyl 2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylate Intermediate 108 (83 mg, 0.12 mmol) were separated by chiral chromatography on a Chiralpak IC column (250×20 mm, 5 μm), eluted with MeCN, at a flow rate of 12 mL/min, and detected at 205, 215 and 254 nm; the first eluted compound was collected and evaporated to give the title compound, Isomer 1 Intermediate 128 (20 mg, 24%); MS (ESI) m/z [M+H]⁺ 688.2;
the second eluted compound was collected and evaporated to give the title compound, Isomer 2 Intermediate 129 (5 mg, 6%); MS (ESI) m/z [M+H]⁺ 688.2; and
the fourth eluted compound was collected and evaporated to give the title compound, Isomer 4 Intermediate 130 (8 mg, 10%); MS (ESI) m/z [M+H]⁺ 688.2.

### Intermediate 131

### Methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 1

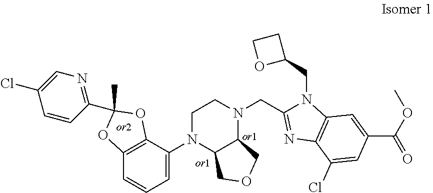 [SMILES: COC(=O)c1cc(Cl)c2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 132

### Methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 2

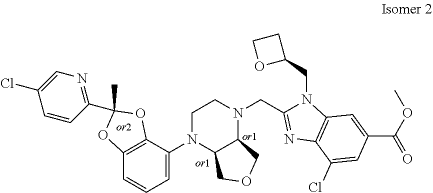 [SMILES: COC(=O)c1cc(Cl)c2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 133

### Methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 3

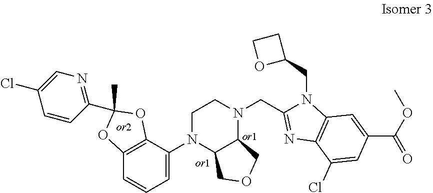 [SMILES: COC(=O)c1cc(Cl)c2nc(CN3CCN(c4cccc5c4O[C@@](C)(c4ccc(Cl)cn4)O5)[C@@H]4COC[C@@H]43)n(C[C@@H]3CCO3)c2c1]

### Intermediate 134

### Methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 4

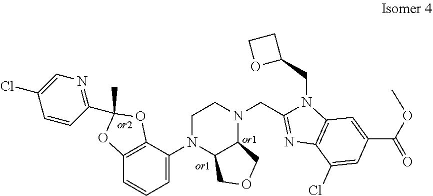 [SMILES: COC(=O)c1cc(Cl)c2nc(CN3CCN(c4cccc5c4O[C@@](C)(c4ccc(Cl)cn4)O5)[C@@H]4COC[C@@H]43)n(C[C@@H]3CCO3)c2c1]

The stereoisomers of methyl 4-chloro-2-(((4aRS,7aSR)-4-(2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate Intermediate 109 (129 mg, 0.194 mmol) were separated by chiral chromatography on a YMC Chiral Art column (250×20 mm, 5 μm), eluted with hexane:IPA:MeOH (70:15:15) at a flow rate of 12 mL/min; the first eluted compound was collected and evaporated to give the title compound Isomer 1, Intermediate 131 (39 mg, 30%); MS (ESI) m/z [M+H]⁺ 668.2;
the second eluted compound mixture was collected and evaporated to give a mixture of isomers; and
the third eluted compound was collected and evaporated to give the title compound Isomer 4, Intermediate 134 (23 mg, 18%); MS (ESI) m/z [M+H]⁺ 666.4.

The stereoisomers of the second eluted compound mixture were separated by chiral chromatography on a Chiralcel OD-H column (250×20 mm, 5 μm), eluted with hexane:IPA:MeOH (70:15:15) at a flow rate of 12 mL/min;
the first eluted compound was collected and evaporated to give the title compound Isomer 2, Intermediate 132 (35 mg, 27%); MS (ESI) m/z [M+H]⁺ 666.2; and
the second eluted compound was collected and evaporated to give the title compound Isomer 3, Intermediate 133 (29 mg, 22%); MS (ESI) m/z [M+H]⁺ 666.2.

### Intermediate 135

### Methyl 4-chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 1

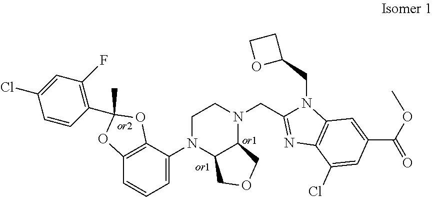 [SMILES: COC(=O)c1cc(Cl)c2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 136

### Methyl 4-chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 2

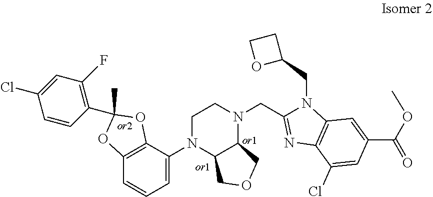 [SMILES: COC(=O)c1cc(Cl)c2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 137

### Methyl 4-chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 3

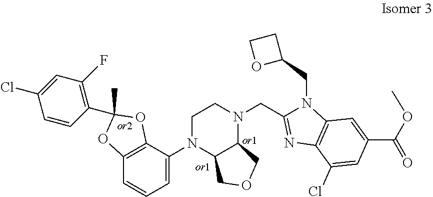 [SMILES: COC(=O)c1cc(Cl)c2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 138

### Methyl 4-chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 4

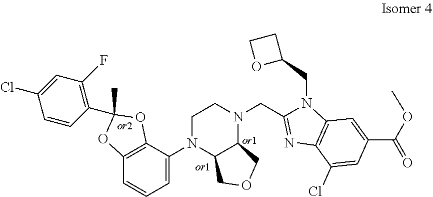 [SMILES: COC(=O)c1cc(Cl)c2nc(CN3CCN(c4cccc5c4O[C@@](C)(c4ccc(Cl)cc4F)O5)[C@@H]4COC[C@@H]43)n(C[C@@H]3CCO3)c2c1]

The stereoisomers of methyl 4-chloro-2-(((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate Intermediate 110 (158 mg, 0.23 mmol) were separated by chiral chromatography on a YMC Chiral Art column (250×20 mm, 5 μm), eluted with hexane:IPA:MeOH (95:5:5), at a flow rate of 13 mL/min; the first eluted compound was collected and evaporated to give the title compound Isomer 1, Intermediate 135 (44 mg, 28%); MS (ESI) m/z [M+H]⁺ 685.2;
the second eluted compound was collected and evaporated to give the title compound Isomer 2, Intermediate 136 (14 mg, 9%); MS (ESI) m/z [M+H]⁺ 685.0;
the third eluted compound was collected and evaporated to give the title compound Isomer 3, Intermediate 137 (46 mg, 29%); MS (ESI) m/z [M+H]⁺ 685.2;
the fourth eluted compound was collected and evaporated to give the title compound Isomer 4, Intermediate 138 (13 mg, 8%); MS (ESI) m/z [M+H]⁺ 683.2;

### Intermediate 139

### Methyl 2-((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 1

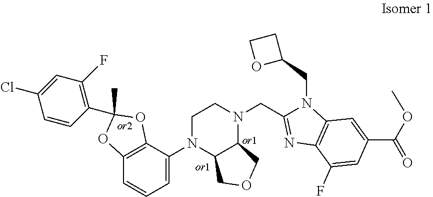 [SMILES: COC(=O)c1cc(F)c2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 140

### Methyl 2-((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 2

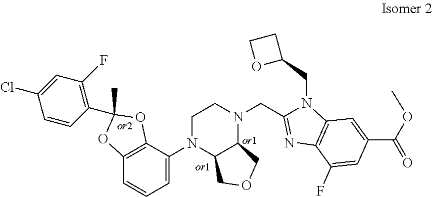 [SMILES: COC(=O)c1cc(F)c2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 141

### Methyl 2-((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 3

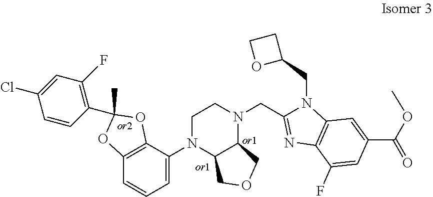 [SMILES: COC(=O)c1cc(F)c2nc(CN3CCN(c4cccc5c4O[C@@](C)(c4ccc(Cl)cc4F)O5)[C@@H]4COC[C@@H]43)n(C[C@@H]3CCO3)c2c1]

### Intermediate 142

### Methyl 2-((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 4

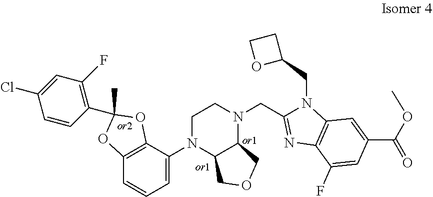 [SMILES: COC(=O)c1cc(F)c2nc(CN3CCN(c4cccc5c4O[C@@](C)(c4ccc(Cl)cc4F)O5)[C@@H]4COC[C@@H]43)n(C[C@@H]3CCO3)c2c1]

The stereoisomers of methyl 2-(((4aRS,7aSR)-4-(2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate Intermediate 111 (114 mg, 0.17 mmol) were separated by chiral chromatography on a ChiralART YMC column (250×20 mm, 5 μm), eluted with hexane:IPA:MeOH (95:5:5), at a flow rate of 13 mL/min; the first eluted compound was collected and evaporated to give the title compound Isomer 1, Intermediate 139 (31 mg, 27%); MS (ESI) m/z [M+H]⁺ 667.2;
the second eluted compound was collected and evaporated to give the title compound Isomer 2, Intermediate 140 (12 mg, 10%); MS (ESI) m/z [M+H]⁺ 667.2;
the third eluted compound was collected and evaporated to give the title compound Isomer 3, Intermediate 141 (28 mg, 24%); MS (ESI) m/z [M+H]⁺ 667.2; and
the fourth eluted compound was collected and evaporated to give the title compound Isomer 4, Intermediate 142 (11 mg, 10%); MS (ESI) m/z [M+H]⁺ 667.2.

### Intermediate 143

### rel-Methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylate, Isomer 1

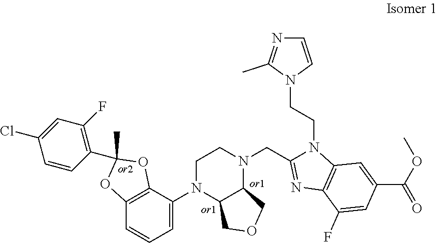 [SMILES: COC(=O)c1cc(F)c2nc(CN3CCN(c4cccc5c4O[C@@](C)(c4ccc(Cl)cc4F)O5)[C@@H]4COC[C@@H]43)n(CCn3ccnc3C)c2c1]

The stereoisomers of rac-methyl 2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylate Intermediate 112 (136 mg, 0.19 mmol) were separated by chiral chromatography on a Chiralpak IC-III column (250×20 mm, 5 μm), eluted with hexane:IPA:MeOH (75:15:15), at a flow rate of 12 mL/min;
the first eluted compound was collected and evaporated to give the title compound Isomer 1, Intermediate 143 (26 mg, 19%); MS (ESI) m/z [M+H]⁺ 705.2.

### Intermediate 144

### rel-Methyl 4-chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylate Isomer 1

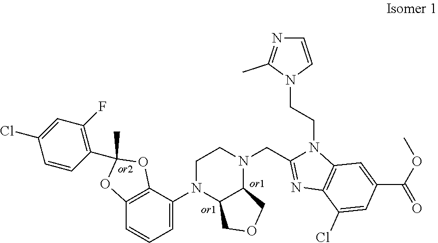 [SMILES: COC(=O)c1cc(Cl)c2nc(CN3CCN(c4cccc5c4O[C@@](C)(c4ccc(Cl)cc4F)O5)[C@@H]4COC[C@@H]43)n(CCn3ccnc3C)c2c1]

The stereoisomers of rac-methyl 4-chloro-2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylate Intermediate 113 (76 mg, 0.11 mmol) were separated by chiral chromatography on a ChiralArt YMC column (250×20 mm, 5 μm), eluted with IPA:MeOH (50:50), at a flow rate of 10 mL/min; the first eluted compound was collected and evaporated to give the title compound Isomer 1, Intermediate 144 (16 mg, 21%); MS (ESI) m/z [M+H]⁺ 721.4.

### Intermediate 145

### rel-Methyl 4-chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylate, Isomer 4

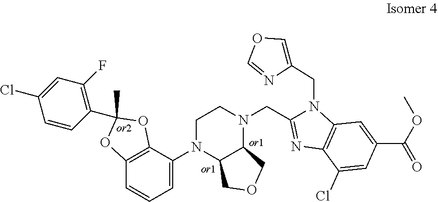 [SMILES: COC(=O)c1cc(Cl)c2nc(CN3CCN(c4cccc5c4O[C@@](C)(c4ccc(Cl)cc4F)O5)[C@@H]4COC[C@@H]43)n(Cc3cocn3)c2c1]

The stereoisomers of rac-methyl 4-chloro-2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylate Intermediate 114 (316 mg, 0.45 mmol) were separated by chiral chromatography on a Chiralpak IA III column (250×20 mm, 5 μm), eluted with hexane:IPA:MeOH (90:5:5), at a flow rate of 18 mL/min;
the fourth eluted compound was collected and evaporated to give the title compound isomer 4, Intermediate 145 (50 mg, 16%); MS (ESI) m/z [M+H]⁺ 694.2.

### Intermediate 146

### rel-Methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylate, Isomer 1

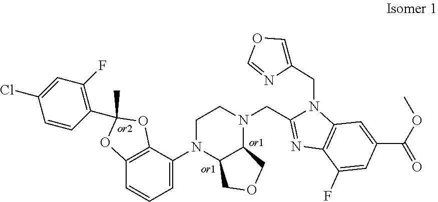 [SMILES: COC(=O)c1cc(F)c2nc(CN3CCN(c4cccc5c4O[C@@](C)(c4ccc(Cl)cc4F)O5)[C@@H]4COC[C@@H]43)n(Cc3cocn3)c2c1]

### Intermediate 147

rel-Methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylate, Isomer 4

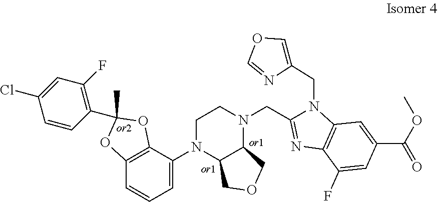 [SMILES: COC(=O)c1cc(F)c2nc(CN3CCN(c4cccc5c4O[C@@](C)(c4ccc(Cl)cc4F)O5)[C@@H]4COC[C@@H]43)n(Cc3cocn3)c2c1]

The stereoisomers of rac-methyl 2-(((4aR,7aS)-4-(2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylate Intermediate 115 (108 mg, 0.16 mmol) were separated by chiral chromatography on a Chiralcel OJ-H column (250×20 mm, 5 μm), eluted with 30% MeOH in CO₂, at a flow rate of 50 mL/min;
the first eluted compound was collected and evaporated to give the title compound Isomer 1, Intermediate 146 (23 mg, 21%); MS (ESI) m/z [M+H]⁺ 678.2; and
the fourth eluted compound was collected and evaporated to give the title compound Isomer 4, Intermediate 147 (10 mg, 9%); MS (ESI) m/z [M+H]⁺ 678.2.

### Intermediate 148

### rel-Methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylate Isomer 1

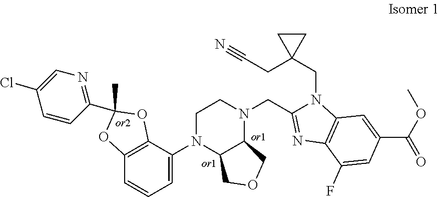 [SMILES: COC(=O)c1cc(F)c2nc(CN3CCN(c4cccc5c4O[C@@](C)(c4ccc(Cl)cn4)O5)[C@@H]4COC[C@@H]43)n(CC3(CC#N)CC3)c2c1]

The stereoisomers of rac-methyl 2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylate Intermediate 116 (252 mg, 0.37 mmol) were separated by chiral chromatography on a CHIRALART YMC column (250×21 mm, 5 μm) eluted with hexane:IPA:MeOH (50:25:25) at a flow rate of 14 mL/min;
the first eluted compound was collected and evaporated to give the title compound Isomer 1 Intermediate 148 (79 mg, 31%); MS (ESI) m/z [M+H]⁺ 673.2.

### Intermediate 149

### rel-Methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylate, Isomer 1

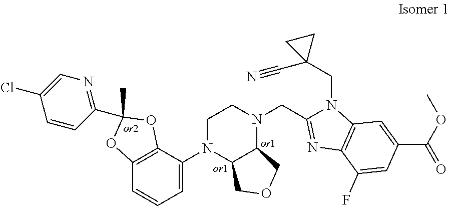 [SMILES: COC(=O)c1cc(F)c2nc(CN3CCN(c4cccc5c4O[C@@](C)(c4ccc(Cl)cn4)O5)[C@@H]4COC[C@@H]43)n(CC3(C#N)CC3)c2c1]

### Intermediate 150

### rel-Methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylate, Isomer 2

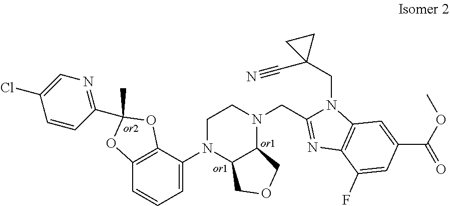 [SMILES: COC(=O)c1cc(F)c2nc(CN3CCN(c4cccc5c4O[C@@](C)(c4ccc(Cl)cn4)O5)[C@@H]4COC[C@@H]43)n(CC3(C#N)CC3)c2c1]

### Intermediate 151

### rel-Methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylate, Isomer 3

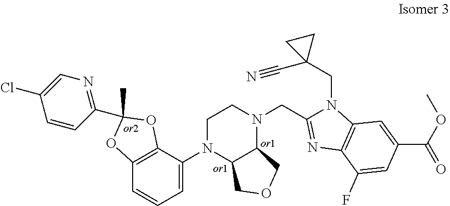 [SMILES: COC(=O)c1cc(F)c2nc(CN3CCN(c4cccc5c4O[C@@](C)(c4ccc(Cl)cn4)O5)[C@@H]4COC[C@@H]43)n(CC3(C#N)CC3)c2c1]

The stereoisomers of rac-methyl 2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylate Intermediate 117 (220 mg, 0.33 mmol) were separated by chiral chromatography on a CHIRALPAK IB column (250×21 mm, 5 μm) eluted with hexane:IPA:MeOH (90:5:5) at a flow rate of 17 mL/min; the first eluted compound was collected and evaporated to give the title compound Isomer 1 Intermediate 149 (61 mg, 28%); MS (ESI) m/z [M+H]⁺ 659.0;
the second eluted compound was collected and evaporated to give the title compound Isomer 2 Intermediate 150 (43 mg, 19%); MS (ESI) m/z [M+H]⁺ 659.0; and
the third eluted compound was collected and evaporated to give the title compound Isomer 3 Intermediate 151 (68 mg, 31%); MS (ESI) m/z [M+H]⁺ 659.0.

### Intermediate 152

### rel-Methyl 4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate Isomer 1

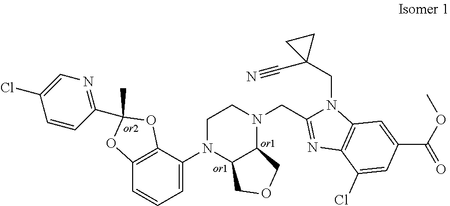 [SMILES: COC(=O)c1cc(Cl)c2nc(CN3CCN(c4cccc5c4O[C@@](C)(c4ccc(Cl)cn4)O5)[C@@H]4COC[C@@H]43)n(CC3(C#N)CC3)c2c1]

### Intermediate 153

### rel-Methyl 4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate Isomer 2

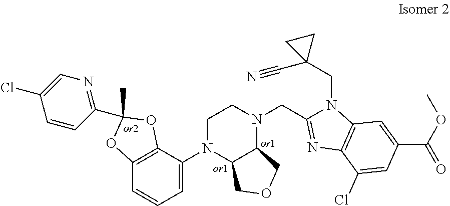 [SMILES: COC(=O)c1cc(Cl)c2nc(CN3CCN(c4cccc5c4O[C@@](C)(c4ccc(Cl)cn4)O5)[C@@H]4COC[C@@H]43)n(CC3(C#N)CC3)c2c1]

The stereoisomers of rac-methyl 4-chloro-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate Intermediate 118 (84 mg, 0.12 mmol) were separated on a chiral chromatography on a CHIRALPAK IB column (250×20 mm, 5 μm) eluted with hexane:IPA:MeOH (80:10:10) at a flow rate of 14 mL/min; the first eluted compound was collected and evaporated to give the title compound Isomer 1, Intermediate 152 (20 mg, 24%); MS (ESI) m/z [M+H]⁺ 675.2; and
the second eluted compound was collected and evaporated to give the title compound Isomer 2 Intermediate 153 (14 mg, 17%); MS (ESI) m/z [M+H]⁺ 675.2.

### Intermediate 154

### rel-Methyl 4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylate Isomer 1

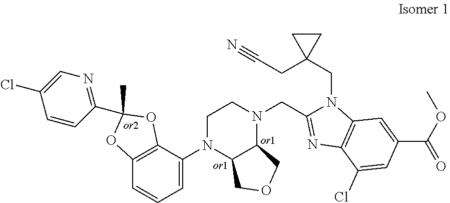 [SMILES: COC(=O)c1cc(Cl)c2nc(CN3CCN(c4cccc5c4O[C@@](C)(c4ccc(Cl)cn4)O5)[C@@H]4COC[C@@H]43)n(CC3(CC#N)CC3)c2c1]

The stereoisomers of rac-methyl 4-chloro-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylate Intermediate 119 (76 mg, 0.11 mmol) were separated by chiral chromatography on a CHIRALPAK IC column (250×20 mm, 5 μm) eluted with hexane:IPA:MeOH (50:25:25) at a flow rate of 12 mL/min; the first eluted compound was collected and evaporated to give the title compound Isomer 1, Intermediate 154 (19 mg, 25%); MS (ESI) m/z [M+H]⁺ 691.0.

### Intermediate 155

### rel-Methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylate Isomer 1

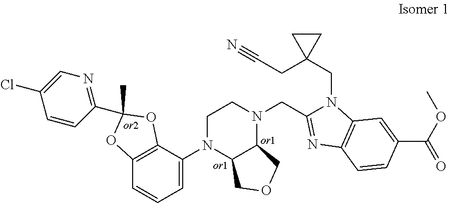 [SMILES: COC(=O)c1ccc2nc(CN3CCN(c4cccc5c4O[C@@](C)(c4ccc(Cl)cn4)O5)[C@@H]4COC[C@@H]43)n(CC3(CC#N)CC3)c2c1]

The stereoisomers of rac-methyl 2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylate Intermediate 120 (500 mg, 0.76 mmol) were separated by chiral chromatography on a Chiralcel OD-H column (250×20 mm, 5 μm) eluted with hexane:IPA:MeOH (70:15:15) at a flow rate of 12 mL/min; The first eluted compound was collected and evaporated to give the title compound Isomer 1 Intermediate 155 (137 mg, 27%); MS (ESI) m/z [M+H]⁺ 655.4.

### Intermediate 156

### rel-Methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate Isomer 1

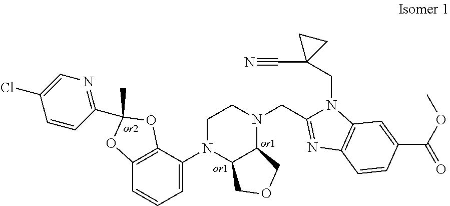 [SMILES: COC(=O)c1ccc2nc(CN3CCN(c4cccc5c4O[C@@](C)(c4ccc(Cl)cn4)O5)[C@@H]4COC[C@@H]43)n(CC3(C#N)CC3)c2c1]

The stereoisomers of rac-methyl 2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate Intermediate 121 (400 mg, 0.62 mmol) were separated by chiral chromatography on a CHIRALPAK IC column (250×21 mm, 5 μm) eluted with hexane:IPA:MeOH (75:15:15) at a flow rate of 14 mL/min; The first eluted compound was collected and evaporated to give the title compound Isomer 1 Intermediate 156 (101 mg, 27%); MS (ESI) m/z [M+H]⁺ 641.2.

### Intermediate 157

### rel-Methyl 1-(2-(1H-pyrazol-1-yl)ethyl)-4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylate Isomer 1

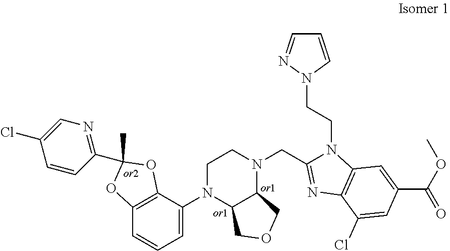 [SMILES: COC(=O)c1cc(Cl)c2nc(CN3CCN(c4cccc5c4O[C@@](C)(c4ccc(Cl)cn4)O5)[C@@H]4COC[C@@H]43)n(CCn3cccn3)c2c1]

The stereoisomers of rac-methyl 1-(2-(1H-pyrazol-1-yl)ethyl)-4-chloro-2-(((4aR,7aS)-4-(2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylate Intermediate 122 (630 mg, 0.91 mmol) were separated by chiral chromatography on a Chiralpak IA column (250×50 mm, 5 μm) eluted with 35% (IPA, 20 mM DEA) in CO₂, 120 bar, at a flow rate of 350 mL/min and detected at 225 nm;
the first eluted compound mixture was collected and evaporated to yield a mixture of isomers (405 mg). The stereoisomers of the first eluted compound mixture were separated by chiral chromatography on a YMC SZ (imob C2) column (250×30 mm, 5 μm), eluted with 35% (MeOH, 20 mM DEA) in CO₂, 120 bar, at a flow rate of 150 mL/min and detected at 220 nm; the first eluted compound was collected and evaporated to give the title compound Isomer 1 Intermediate 157 (130 mg, 20%); MS (ESI) m/z [M+H]⁺ 692.5.

### Intermediate 158

### tert-Butyl ((3R,4S)-4-((2,4,5-trimethoxybenzyl)amino)tetrahydrofuran-3-yl)carbamate

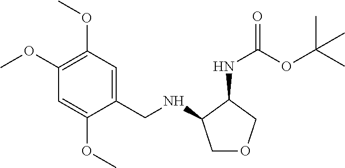 [SMILES: COc1cc(OC)c(OC)cc1CN[C@@H]1COC[C@@H]1NC(=O)OC(C)(C)C]

2,4,5-Trimethoxybenzaldehyde (9.70 g, 49.44 mmol) was added to a solution of tert-butyl ((3R,4S)-4-aminotetrahydrofuran-3-yl)carbamate (10 g, 49.44 mmol) in MeOH (100 mL) and the reaction mixture was stirred at 80° C. for 15 min. TFA (3.81 mL, 49.44 mmol) and NaBH₃CN (2.55 g, 40.54 mmol) were added and the reaction mixture was stirred at 30° C. for 5 h. The solvent was removed under reduced pressure and the residue was diluted with water (100 mL). The aqueous layer was extracted with DCM (3×100 mL), and the combined organic layer was dried over Na₂SO₄, filtered and evaporated. The crude product was purified by flash chromatography on silica (60-70% EtOAc in petroleum ether) to give the title compound (16.00 g, 85%) as a pale yellow gum; MS (ESI) m/z [M+H]⁺ 383.

### Intermediate 159

### (3S,4R)—N³-(2,4,5-Trimethoxybenzyl)tetrahydrofuran-3,4-diamine

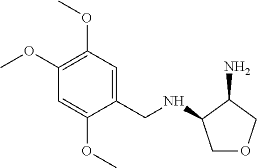 [SMILES: COc1cc(OC)c(OC)cc1CN[C@@H]1COC[C@@H]1N]

4 M HCl in 1,4-dioxane (52.3 mL, 209.17 mmol) was added to a solution of tert-butyl ((3R,4S)-4-((2,4,5-trimethoxybenzyl)amino)tetrahydrofuran-3-yl)carbamate Intermediate 158 (16 g, 41.83 mmol) in DCM (150 mL) and the reaction mixture was stirred at 30° C. for 3 h. The reaction mixture was concentrated under reduced pressure to give the title compound (11.5 g, 97%) as a pale yellow solid; MS (ESI) m/z [M+H]⁺ 283. The product was used in the next step directly without further purification.

### Intermediate 160

### (4aR,7aS)-1-(2,4,5-Trimethoxybenzyl)tetrahydrofuro[3,4-b]pyrazine-2,3(1H,4H)-dione

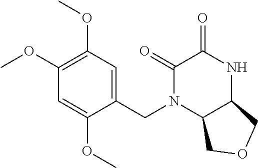 [SMILES: COc1cc(OC)c(OC)cc1CN1C(=O)C(=O)N[C@H]2COC[C@H]21]

Dimethyloxalate (7.21 g, 61.10 mmol) was added to a solution of (3S,4R)—N³-(2,4,5-Trimethoxybenzyl)tetrahydrofuran-3,4-diamine Intermediate 159 (11.5 g, 40.73 mmol) and Et₃N (17.03 mL, 122.19 mmol) in MeOH (150 mL) and the reaction mixture was stirred at 80° C. for 18 h. The solvent was removed under reduced pressure. The crude product was purified by flash chromatography on silica (70-80% MeOH in DCM) to give the title compound (12.50 g, 91%) as a pale yellow oil; MS (ESI) m/z [M+H]⁺ 337.

### Intermediate 161

### (4aR,7aS)-1-(2,4,5-Trimethoxybenzyl)octahydrofuro[3,4-b]pyrazine

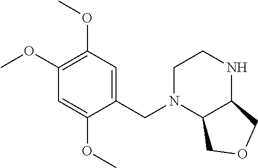 [SMILES: COc1cc(OC)c(OC)cc1CN1CCN[C@H]2COC[C@H]21]

2.5 M LiAlH₄ in THF (47.6 mL, 118.93 mmol) was added dropwise to a solution of (4aR,7aS)-1-(2,4,5-trimethoxybenzyl)tetrahydrofuro[3,4-b]pyrazine-2,3(1H,4H)-dione Intermediate 160 (12.5 g, 37.16 mmol) in THF (150 mL) at 0° C. The reaction mixture was stirred at 15° C. for 8 h. The reaction mixture was quenched with Na₂SO₄ decahydrate (40 g), and the mixture was filtered. The filtrate was collected and evaporated at reduced pressure to give the title compound (11.00 g, 96%) as a yellow oil; MS (ESI) m/z [M+H]⁺ 309. The product was used in the next step directly without further purification.

### Intermediate 162

### tert-Butyl (4aS,7aR)-4-(2,4,5-trimethoxybenzyl)hexahydrofuro[3,4-b]pyrazine-1(2H)-carboxylate

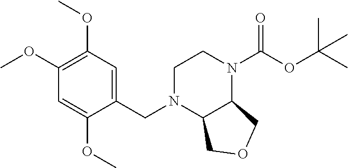 [SMILES: COc1cc(OC)c(OC)cc1CN1CCN(C(=O)OC(C)(C)C)[C@H]2COC[C@H]21]

Di-tert-butyl dicarbonate (15.57 g, 71.34 mmol) was added to a solution of (4aR,7aS)-1-(2,4,5-trimethoxybenzyl)octahydrofuro[3,4-b]pyrazine Intermediate 161 (11 g, 35.67 mmol) in EtOH (110 mL) at 30° C., and the reaction mixture was stirred at 15° C. for 3 h. The solvent was removed under reduced pressure and the crude product was purified by flash chromatography on silica (60-70% EtOAc in petroleum ether) to give the title compound (11.00 g, 75%) as a pale yellow oil; MS (ESI) m/z [M+H]⁺ 409.

### Intermediate 163

### tert-Butyl (4aS,7aR)-hexahydrofuro[3,4-b]pyrazine-1(2H)-carboxylate

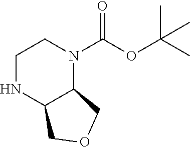 [SMILES: CC(C)(C)OC(=O)N1CCN[C@@H]2COC[C@@H]21]

Pd/C (10%, 1.303 g, 1.22 mmol) was added to a solution of tert-butyl (4aS,7aR)-4-(2,4,5-trimethoxybenzyl)hexahydrofuro[3,4-b]pyrazine-1(2H)-carboxylate Intermediate 162 (5 g, 12.24 mmol) in THF (50 mL) at 30° C. and the resulting mixture was hydrogenated under an atmosphere of H₂ (g) (1 atm) at 15° C. for 30 h. The reaction mixture was filtered through celite and washed with MeOH (3×300 mL). The solvent was removed under reduced pressure the crude product was purified by flash chromatography on silica (70-80% EtOAc in petroleum ether) to give the title compound (2.0 g, 72%) as a pale yellow solid; MS (ESI) m/z [M+H]⁺ 229.

### Intermediate 164

### (S)-2-(4-Bromo-2-methylbenzo[d][3]dioxol-2-yl)-5-chloropyridine

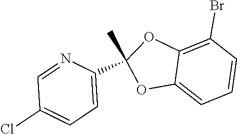 [SMILES: C[C@]1(c2ccc(Cl)cn2)Oc2cccc(Br)c2O1]

The stereoisomers of 2-(4-bromo-2-methylbenzo[d][1,3]dioxol-2-yl)-5-chloropyridine WO2020234726 (6 g, 18.37 mmol) were separated by chiral chromatography on an LUX A1 (AD) column (250×30 mm, 5 μm), eluted with 3% (IPA, 20 mM DEA) in CO₂, 120 bar, at a flow rate of 150 mL/min and detected at 220 nm;

The first eluted compound was collected and evaporated to give the title compound, Intermediate 164 (2 g, 33%); [α]_{D}²⁰+152 (c 1.00, MeCN); ¹H NMR (400 MHz, DMSO-d₆) δ 2.07 (3H, d), 6.83 (1H, td), 6.97 (1H, d), 7.06 (1H, d), 7.64 (1H, dd), 8.04 (1H, dd), 8.74 (1H, d).

The absolute configuration of Intermediate 164 was determined by vibrational circular dichroism (VCD) spectroscopy. The experimental spectrum recorded in CDCl₃ was compared to a simulated spectrum of the (S) enantiomer calculated using density functional theory at the B3PW91/cc-pVTZ level of theory. Based on the large number of points of agreement between the experimental and simulated spectra, the title compound was assigned as the (S) enantiomer.

### Intermediate 165

### rac-1-Benzyl 4-(tert-butyl) (4aR,7aS)-hexahydrofuro[3,4-b]pyrazine-1,4-dicarboxylate

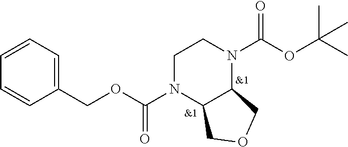 [SMILES: CC(C)(C)OC(=O)N1CCN(C(=O)OCc2ccccc2)[C@@H]2COC[C@@H]21]

TEA (26.6 g, 0.262 mol), followed by a solution of benzyl chloroformate (29.8 g, 0.175 mol) in dry DCM (70 mL), was added to a solution of rac-tert-butyl (4aS,7aR)-hexahydrofuro[3,4-b]pyrazine-1(2H)-carboxylate (40 g, 0.175 mol) in dry DCM (150 mL) and the reaction mixture was stirred at rt overnight. The reaction mixture was diluted with DCM (50 mL) and washed with 10% citric acid (70 mL), sat NaHCO₃ (70 mL) and brine (100 mL). The organic layer was dried over anhydrous Na₂SO₄, filtered and concentrated under reduced pressure. The crude product was purified by flash chromatography (hexane:MTBE, 1:1) to give the title compound (63 g, 98%); ¹H NMR (400 MHz, CDCl₃) δ 7.41-7.17 (m, 5H), 5.16-5.01 (m, 2H), 4.54 (d, 2H), 3.98 (dd, 2H), 3.87-3.57 (m, 4H), 3.35 (ddt, 2H), 1.42 (d, 9H).

### Intermediate 166

### rel-1-Benzyl 4-(tert-butyl) (4aR,7aS)-hexahydrofuro[3,4-b]pyrazine-1,4-dicarboxylate Isomer 1

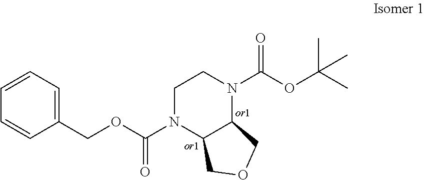 [SMILES: CC(C)(C)OC(=O)N1CCN(C(=O)OCc2ccccc2)[C@@H]2COC[C@@H]21]

### Intermediate 167

### rel-1-Benzyl 4-(tert-butyl) (4aR,7aS)-hexahydrofuro[3,4-b]pyrazine-1,4-dicarboxylate Isomer 2

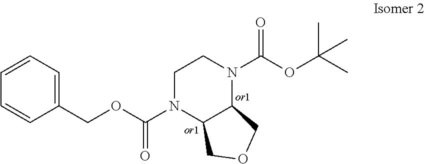 [SMILES: CC(C)(C)OC(=O)N1CCN(C(=O)OCc2ccccc2)[C@@H]2COC[C@@H]21]

The stereoisomers of rac-1-benzyl 4-(tert-butyl) (4aR,7aS)-hexahydrofuro[3,4-b]pyrazine-1,4-dicarboxylate Intermediate 165 were separated by chiral chromatography on a CHIRALPAK IA column (250×30 mm, 5 μm), eluted with hexane:IPA:MeOH (70:15:15), at a flow rate of 25 mL/min;

The first eluted compound was collected and evaporated to give the title compound Isomer 1, Intermediate 166 (24.95 g); MS (ESI) m/z [(M-t-Bu)+H]⁺ 307.2 [(M-Boc)+H]⁺ 263.2.

The second eluted compound was collected and evaporated to give the title compound Isomer 2, Intermediate 167; (24.68 g) MS (ESI) m/z [(M-t-Bu)+H]⁺ 307.2 [(M-Boc)+H]⁺ 263.2.

### Intermediate 168

### tert-Butyl (4aS,7aR)-hexahydrofuro[3,4-b]pyrazine-1(2H)-carboxylate

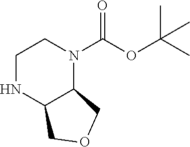 [SMILES: CC(C)(C)OC(=O)N1CCN[C@@H]2COC[C@@H]21]

A solution of rel-1-benzyl 4-(tert-butyl) (4aR,7aS)-hexahydrofuro[3,4-b]pyrazine-1,4-dicarboxylate Isomer 2 Intermediate 167 (24.68 g 0.068 mol) and Pd/C (10%, 2.4 g) in MeOH (100 mL) was degassed under vacuum and then purged with H₂(g) (×3). The reaction mixture was then stirred at rt under an atmosphere of H₂(g) (2 atm) until complete reaction (monitoring by NMR). The catalyst was carefully removed by filtration and washed with MeOH (2×20 mL). The filtrate was concentrated in vacuo to give the title compound Intermediate 168 (14.5 g, 93%); [α]_{D}²⁰+74.12 (c 0.5, MeOH); ¹H NMR (500 MHz, CDCl₃) δ 4.48 (d, J=89.8 Hz, 1H), 4.00-3.64 (m, 4H), 3.26 (t, J=4.6 Hz, 1H), 2.94 (d, J=46.6 Hz, 2H), 2.72 (td, J=12.3, 3.2 Hz, 1H), 1.79 (s, 2H), 1.44 (s, 9H).

The absolute configuration of the title compound was determined by converting the title compound in two steps to the 4-chlorobenzoyl derivative Intermediate 170 as described below in the experimental descriptions for Intermediate 169 and Intermediate 170. Based on the X-ray data of Intermediate 170 the title compound Intermediate 168 was assigned as the (4aS, 7aR) enantiomer.

### Intermediate 169

### rel-1-(tert-Butyl) 4-(4-chlorobenzyl) (4aR,7aS)-hexahydrofuro[3,4-b]pyrazine-1,4-dicarboxylate Isomer 2

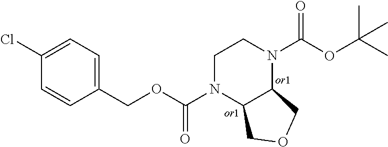 [SMILES: CC(C)(C)OC(=O)N1CCN(C(=O)OCc2ccc(Cl)cc2)[C@@H]2COC[C@@H]21]

TEA (115 mg, 1.1 mmol) followed by a solution of 4-chlorobenzyl carbonochloridate (173 mg, 0.75 mmol) in dry DCM (5 mL) were added to a solution of tert-butyl (4aS,7aR)-hexahydrofuro[3,4-b]pyrazine-1(2H)-carboxylate Intermediate 168 (173 mg, 0.75 mmol) in dry DCM (10 mL) and the reaction mixture was stirred at rt overnight. The reaction mixture was diluted with DCM (5 mL) and washed with 10% citric acid (10 mL), sat NaHCO₃ (20 mL) and brine (10 mL). The organic layer was dried over anhydrous Na₂SO₄, filtered and concentrated under reduced pressure. The crude product was purified by flash chromatography (hexane:EtOAc, 1:1) to give the title compound (175 mg, 58%); MS (ESI) m/z [M-Boc+H]⁺ 297.0.

### Intermediate 170

### 4-Chlorobenzyl (4aR,7aS)-hexahydrofuro[3,4-b]pyrazine-1(2H)-carboxylate, Isomer 2

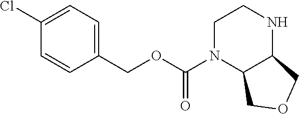 [SMILES: O=C(OCc1ccc(Cl)cc1)N1CCN[C@H]2COC[C@H]21]

2 M HCl in Et₂O (2 mL) was added to a solution rel-1-(tert-butyl) 4-(4-chlorobenzyl) (4aR,7aS)-hexahydrofuro[3,4-b]pyrazine-1,4-dicarboxylate Isomer 2 Intermediate 169 (99 mg, 0.249 mmol) in DCM (15 mL). The solution was stirred at ambient temperature for 8 h and then concentrated under reduced pressure to give the HCl-salt of the title compound (85 mg, quantitative yield) as a white solid; MS (ESI) m/z [M+H]⁺ 297.2.

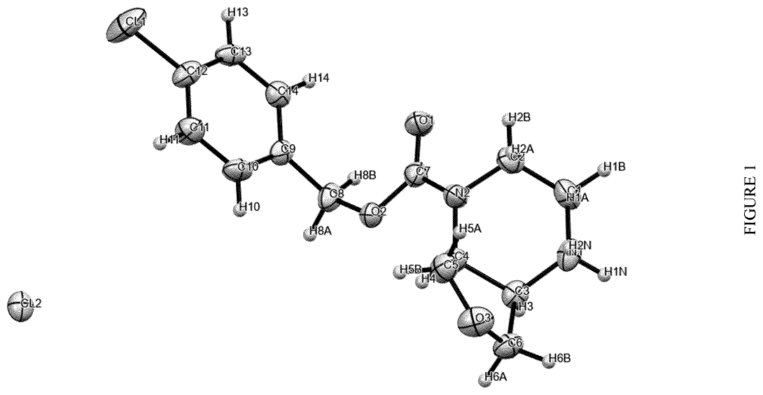

Crystals for X-ray diffraction studies from the title compound was grown from a mixture of EtOAc:MeCN (2:1). The molecular structure of Intermediate 170 is shown in FIG. 1. Crystallographic data: C₁₄H₁₈ClN₂O₃, Cl, M=396.87, monoclinic, space group P2₁, Cell Lengths: a=7.6548(3), b=7.4219(2), c=14.0539(5) Å, cell angles: α 90 β 97.735(2) γ 90, cell volume: V=791.182, crystal size ca. 0.1×0.23×0.49 mm, R-factor (%) 3.85.

### Intermediate 171

### tert-Butyl (4aS,7aR)-4-((S)-2-(5-chloropyridin-2-yl)-2-methylbenzo[d][1,3]dioxol-4-yl)hexahydrofuro[3,4-b]pyrazine-1(2H)-carboxylate

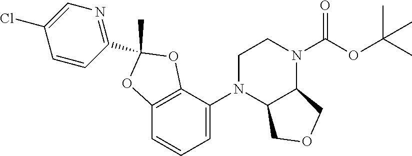 [SMILES: CC(C)(C)OC(=O)N1CCN(c2cccc3c2O[C@@](C)(c2ccc(Cl)cn2)O3)[C@@H]2COC[C@@H]21]

A mixture of(S)-2-(4-bromo-2-methylbenzo[d][1,3]dioxol-2-yl)-5-chloropyridine Intermediate 164 (40.8 g, 124.84 mmol), tert-butyl (4aS,7aR)-hexahydrofuro[3,4-b]pyrazine-1(2H)-carboxylate Intermediate 168 (30 g, 131.41 mmol), BINAP (4.91 g, 7.88 mmol) and Palladium(π-cinnamyl) chloride dimer (2.042 g, 3.94 mmol) in cyclopentyl methyl ether (700 mL) was evacuated and backfilled with N₂(g) (×5). Sodium tert-butoxide (25.3 g, 262.82 mmol) was added and the reaction mixture was evacuated and backfilled with N₂(g) (×3) and then stirred at 45° C. for 24 h under an atmosphere of N₂(g). The reaction mixture was washed with brine (2×100 mL) and the organic layer was dried over MgSO₄, filtered and evaporated at reduced pressure. The crude product was purified by flash chromatography (5-30% EtOAc in heptane). The product containing fractions were collected and concentrated at reduced pressure and the crude compound was purified by flash chromatography (5-30% EtOAc in heptane). The product was dissolved in EtOAc (300 mL), SiliaMetS Thiol (30 g, 40-63 μm) was added and the mixture was stirred at rt for 2 h, and then filtered through celite. The filtrate was collected and concentrated at reduced pressure to give the title compound (38.0 g, 61%); [α]_{D}²⁰+126 (c 1.0, MeCN); MS (ESI) m/z [M+H]⁺ 474.1; ¹H NMR (500 MHz, DMSO, 25° C.) δ 1.41 (9H, s), 2.03 (3H, s), 2.69-2.78 (1H, m), 3.14-3.25 (1H, m), 3.4-3.47 (1H, m), 3.52-3.58 (1H, m), 3.69-3.8 (3H, m), 3.82-3.86 (1H, m), 3.90 (1H, dd), 4.5-4.6 (1H, m), 6.43-6.49 (1H, m), 6.62-6.67 (1H, m), 6.78 (1H, t), 7.61-7.65 (1H, m), 8.01 (1H, dd), 8.71 (1H, d).

### Intermediate 172

### (4aR,7aS)-1-((S)-2-(5-Chloropyridin-2-yl)-2-methylbenzo[d][1,3]dioxol-4-yl)octahydrofuro[3,4-b]pyrazine

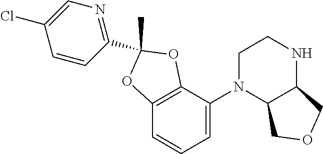 [SMILES: C[C@]1(c2ccc(Cl)cn2)Oc2cccc(N3CCN[C@H]4COC[C@H]43)c2O1]

pTsOH hydrate (32.7 g, 171.75 mmol) was added to a solution of tert-butyl (4aS,7aR)-4-((S)-2-(5-chloropyridin-2-yl)-2-methylbenzo[d][1,3]dioxol-4-yl)hexahydrofuro[3,4-b]pyrazine-1(2H)-carboxylate Intermediate 171 (37 g, 78.07 mmol) in EtOAc (80 mL) and the reaction mixture was stirred at 55° C. for 90 min. The reaction mixture was cooled to rt, diluted with EtOAc (100 mL) and sat K₂CO₃ (aq, 10 mL) was added dropwise. The organic layer was washed with sat K₂CO₃ (aq. 3×50 mL), dried over MgSO₄, filtered and concentrated at reduced pressure to give the title compound (23.60 g, 81%) as an off-white solid; MS (ESI) m/z [M+H]⁺ 374.2.

### Intermediate 173

### Methyl 2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate

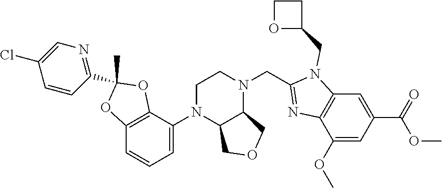 [SMILES: COC(=O)c1cc(OC)c2nc(CN3CCN(c4cccc5c4O[C@@](C)(c4ccc(Cl)cn4)O5)[C@@H]4COC[C@@H]43)n(C[C@@H]3CCO3)c2c1]

K₂CO₃ (8.72 g, 63.13 mmol) and methyl (S)-2-(chloromethyl)-4-methoxy-1-(oxetan-2-ylmethyl)-1H-benzo[d]imidazole-6-carboxylate Intermediate 3 (10.76 g, 33.14 mmol) were added to a solution of (4aR,7aS)-1-((S)-2-(5-chloropyridin-2-yl)-2-methylbenzo[d][1,3]dioxol-4-yl)octahydrofuro[3,4-b]pyrazine Intermediate 172 (11.8 g, 31.56 mmol) in MeCN (100 mL) and the reaction mixture was heated at 60° C. for 18 h. The reaction mixture was cooled to rt and EtOAc (50 mL) was added. The organic layer was washed with NaHCO₃ (aq, 2×50 mL), dried over MgSO₄, filtered and concentrated at reduced pressure. The crude compound was purified by preparative HPLC, PrepMethod H (gradient: 35-100%). Relevant fractions were combined and the mixture was concentrated at reduced pressure. The water layer was extracted with EtOAc (2×30 mL). The combined organic layer was dried over MgSO₄, filtered, and the filtrate was stirred with SiliaMetS Thiol (13 g, 40-63 μm) at rt for 20 h. The mixture was filtered and the filtrate was concentrated at reduced pressure. The crude compound was purified by flash chromatography (30-100% EtOAc in heptane) to give the title compound (15.00 g, 71.8%) as a colourless oil; MS (ESI) m/z [M+H]⁺ 662.3.

### EXAMPLES

### Example 1a

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid, Isomer 1

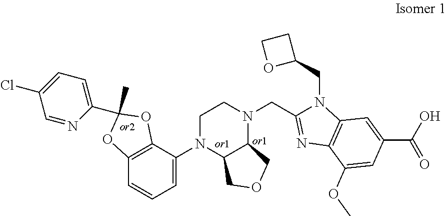 [SMILES: COc1cc(C(=O)O)cc2c1nc(CN1CCN(c3cccc4c3O[C@@](C)(c3ccc(Cl)cn3)O4)[C@@H]3COC[C@@H]31)n2C[C@@H]1CCO1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 1 Intermediate 24 (23 mg, 0.03 mmol) and 1,3,4,6,7,8-hexahydro-2H-pyrimido[1,2-a]pyrimidine (10 mg, 0.07 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, dissolved in DMSO, filtered and purified by preparative HPLC, PrepMethod A, (gradient: 20-90%) to give the title compound (12 mg, 52%); HRMS (ESI) m/z [M+H]⁺ calcd for C₃₃H₃₅ClN₅O₇: 648.2220, found: 648.2234; ¹H NMR (500 MHz, CD₃OD, 25° C.) δ 2.04 (3H, s), 2.44-2.52 (1H, m), 2.57-2.65 (1H, m), 2.72-2.82 (2H, m), 3.02-3.07 (1H, m), 3.15-3.21 (2H, m), 3.39 (1H, t), 3.53 (1H, d), 3.77-3.84 (1H, m), 3.96 (1H, dd), 4.03 (3H, s), 4.38-4.5 (2H, m), 4.54 (1H, d), 4.57-4.63 (1H, m), 4.65-4.72 (1H, m), 4.79-4.84 (1H, m), 5.16-5.25 (2H, m), 6.42 (1H, dd), 6.51 (1H, dd), 6.74 (1H, t), 7.44 (1H, d), 7.66 (1H, dd), 7.89 (1H, dd), 7.92 (1H, d), 8.61 (1H, dd).

### Example 1b

### 2-((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid, Isomer 3

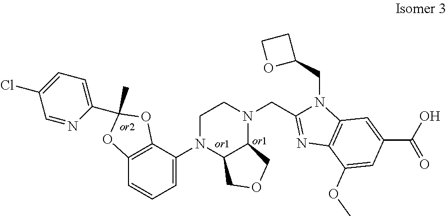 [SMILES: COc1cc(C(=O)O)cc2c1nc(CN1CCN(c3cccc4c3O[C@@](C)(c3ccc(Cl)cn3)O4)[C@@H]3COC[C@@H]31)n2C[C@@H]1CCO1]

A mixture or methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 3 Intermediate 25 (35 mg, 0.05 mmol) and 1,3,4,6,7,8-hexahydro-2H-pyrimido[1,2-a]pyrimidine (15 mg, 0.11 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The mixture was concentrated at reduced pressure, dissolved in DMSO, filtered and the residue was purified by preparative HPLC, PrepMethod A, (gradient: 20-90%) to give the title compound (19 mg, 57%); HRMS (ESI) m/z [M+H]⁺ calcd for C₃₃H₃₅ClN₅O₇: 648.2220, found: 648.2224; ¹H NMR (500 MHz, CD₃OD) 2.03 (3H, s), 2.36 (1H, dq), 2.48 (1H, td), 2.66-2.73 (1H, m), 2.73-2.82 (1H, m), 3.05 (1H, s), 3.11-3.2 (2H, m), 3.36 (1H, t), 3.60 (1H, d), 3.73 (1H, t), 3.86 (1H, dd), 4.05 (3H, s), 4.17 (1H, dt), 4.23 (1H, d), 4.55 (3H, td), 4.72 (1H, td), 5.04 (1H, dd), 5.23-5.32 (1H, m), 6.39 (1H, d), 6.50 (1H, d), 6.73 (1H, t), 7.44-7.47 (1H, m), 7.64 (1H, d), 7.87 (1H, dd), 7.94 (1H, d), 8.08 (1H, s), 8.59 (1H, d).

### Example 1b, Alternative Preparation

### 2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid

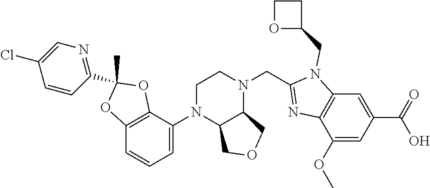 [SMILES: COc1cc(C(=O)O)cc2c1nc(CN1CCN(c3cccc4c3O[C@@](C)(c3ccc(Cl)cn3)O4)[C@@H]3COC[C@@H]31)n2C[C@@H]1CCO1]

A solution of LiOH (1.356 g, 56.63 mmol) in water (20 mL) was added slowly over 5 min to a solution of methyl 2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate Intermediate 173 (15 g, 22.65 mmol) in THF (40 mL) and the resulting mixture was stirred at 35° C. overnight. The reaction mixture was concentrated at reduced pressure. Water (300 mL) and EtOAc (100 mL) was added to the crude product and the mixture was stirred. The pH of the solution was adjusted to pH 4.1 by addition of 1 M citric acid. The layers were separated and the aqueous layer was extracted with EtOAc (2×50 mL). The combined organic layer was washed with water (2×20 mL), dried over MgSO₄ and filtered. The filtrate was collected and concentrated to half the volume and the mixture was stirred at rt. After 1 h, a solid was formed and the stirring was continued for 3 days. The solids were isolated by filtration and dried in vacuo for 72 h to give the title compound (11.00 g, 74.9%); [α]_{D}²⁰+72 (c 1.0, MeCN); HRMS (ESI) m/z [M+H]⁺ calcd for C₃₃H₃₅ClN₅O₇: 648.2220, found: 648.2240; ¹H NMR (500 MHz, DMSO-d₆, 25° C.) δ 2.02 (3H, s), 2.2-2.3 (1H, m), 2.34-2.44 (1H, m), 2.56-2.64 (1H, m), 2.69-2.75 (1H, m), 3.02-3.15 (3H, m), 3.32-3.37 (1H, m), 3.53-3.64 (2H, m), 3.72-3.78 (1H, m), 3.97 (3H, s), 4.05-4.09 (1H, m), 4.1-4.17 (1H, m), 4.36-4.48 (2H, m), 4.49-4.57 (1H, m), 4.57-4.65 (1H, m), 4.76-4.83 (1H, m), 5.1-5.18 (1H, m), 6.37-6.43 (1H, m), 6.5-6.55 (1H, m), 6.73 (1H, t), 7.25-7.29 (1H, m), 7.58-7.63 (1H, m), 7.89 (1H, d), 8.01 (1H, dd), 8.67-8.71 (1H, m), 12.82 (1H, s).

¹H NMR (500 MHz, CD₃OD, 25° C.) δ 2.03 (3H, s), 2.35 (1H, ddt), 2.47 (1H, ddd), 2.63-2.8 (2H, m), 3.04 (1H, ddd), 3.11-3.17 (2H, m), 3.35 (1H, t), 3.59 (1H, d), 3.68-3.76 (1H, m), 3.86 (1H, dd), 4.04 (3H, s), 4.16 (1H, dt), 4.24 (1H, dd), 4.51-4.6 (3H, m), 4.73 (1H, ddd), 5.05 (1H, dd), 5.28 (1H, tdd), 6.39 (1H, dd), 6.49 (1H, dd), 6.73 (1H, t), 7.45 (1H, d), 7.64 (1H, dd), 7.87 (1H, dd), 7.94 (1H, d), 8.59 (1H, dd).

By comparison of ¹H NMR spectra and biological assay data, the title compound 2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid was concluded to be identical to 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid, Isomer 3.

### Example 2a

### 2-((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid, Isomer 3

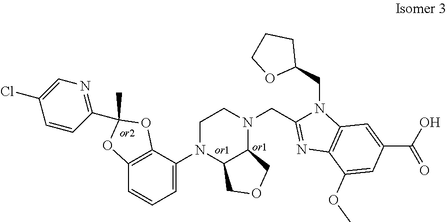 [SMILES: COc1cc(C(=O)O)cc2c1nc(CN1CCN(c3cccc4c3O[C@@](C)(c3ccc(Cl)cn3)O4)[C@@H]3COC[C@@H]31)n2C[C@@H]1CCCO1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylate, Isomer 3 Intermediate 27 (36 mg, 0.05 mmol) and 1,3,4,6,7,8-hexahydro-2H-pyrimido[1,2-a]pyrimidine (15 mg, 0.11 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and filled with N₂(g) (×3) and then stirred at rt overnight. The reaction mixture was concentrated in vacuo, dissolved in DMSO, filtered and purified by preparative HPLC, PrepMethod A, (gradient: 20-90%) to give the title compound (24 mg, 69%); HRMS (ESI) m/z [M+H]⁺ calcd for C₃₄H₃₇ClN₅O₇: 662.2376, found: 662.2384; ¹H NMR (500 MHz, CD₃OD) 1.54-1.63 (1H, m), 1.68-1.78 (1H, m), 1.79-1.88 (1H, m), 2.02 (3H, s), 2.04-2.11 (1H, m), 2.42-2.5 (1H, m), 2.73-2.8 (1H, m), 3.04-3.2 (3H, m), 3.37 (1H, t), 3.62 (1H, d), 3.65-3.75 (2H, m), 3.76-3.83 (1H, m), 3.89 (1H, dd), 4.02 (3H, s), 4.23-4.33 (2H, m), 4.36 (1H, dd), 4.43 (1H, d), 4.65-4.72 (1H, m), 4.93 (1H, dd), 6.34-6.42 (1H, m), 6.47-6.51 (1H, m), 6.72 (1H, t), 7.4-7.45 (1H, m), 7.61-7.64 (1H, m), 7.85 (1H, dd), 7.91 (1H, d), 8.56-8.61 (1H, m).

### Example 2b

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid, Isomer 4

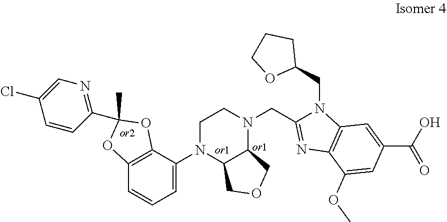 [SMILES: COc1cc(C(=O)O)cc2c1nc(CN1CCN(c3cccc4c3O[C@@](C)(c3ccc(Cl)cn3)O4)[C@@H]3COC[C@@H]31)n2C[C@@H]1CCCO1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylate, Isomer 4 Intermediate 28 (22 mg, 0.03 mmol) and 1,3,4,6,7,8-hexahydro-2H-pyrimido[1,2-a]pyrimidine (9 mg, 0.07 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, dissolved in DMSO, filtered and purified by preparative HPLC, PrepMethod A, (gradient: 20-90%) to give the title compound (15 mg, 68%); HRMS (ESI) m/z [M+H]⁺ calcd for C₃₄H₃₇ClN₅O₇: 662.2376, found: 662.2372; ¹H NMR (500 MHz, CD₃OD) 1.56-1.67 (1H, m), 1.7-1.79 (1H, m), 1.81-1.91 (1H, m), 2.01 (3H, s), 2.10 (1H, d), 2.43-2.52 (1H, m), 2.74-2.81 (1H, m), 3.07-3.12 (1H, m), 3.13-3.24 (2H, m), 3.64 (1H, d), 3.68-3.77 (2H, m), 3.77-3.83 (1H, m), 3.84-3.93 (2H, m), 4.03 (3H, s), 4.25-4.35 (2H, m), 4.39 (1H, dd), 4.45 (1H, d), 4.61-4.68 (1H, m), 4.97 (1H, dd), 6.39-6.45 (1H, m), 6.48 (1H, dd), 6.73 (1H, t), 7.43 (1H, d), 7.65 (1H, dd), 7.86 (1H, dd), 7.92 (1H, d), 8.59 (1H, dd).

### Example 3a

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid, Isomer 1

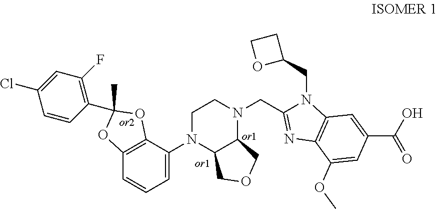 [SMILES: COc1cc(C(=O)O)cc2c1nc(CN1CCN(c3cccc4c3O[C@@](C)(c3ccc(Cl)cc3F)O4)[C@@H]3COC[C@@H]31)n2C[C@@H]1CCO1]

A mixture of methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 1 Intermediate 30 (58 mg, 0.09 mmol) and 1,3,4,6,7,8-hexahydro-2H-pyrimido[1,2-a]pyrimidine (24 mg, 0.17 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, dissolved in DMSO, filtered and purified by preparative HPLC, PrepMethod A, (gradient: 20-90%) to give the title compound (44 mg, 77%); HRMS (ESI) m/z [M+H]⁺ calcd for C₃₄H₃₅ClFN₄O₇: 665.2172, found: 665.2212; ¹H NMR (500 MHz, CD₃OD): 2.05 (3H, s), 2.3-2.41 (1H, m), 2.44-2.52 (1H, m), 2.66-2.74 (1H, m), 2.74-2.81 (1H, m), 3.02-3.07 (1H, m), 3.11-3.16 (2H, m), 3.32-3.37 (1H, m), 3.60 (1H, d), 3.72 (1H, t), 3.85 (1H, dd), 4.05 (3H, s), 4.13-4.2 (1H, m), 4.23-4.29 (1H, m), 4.52-4.61 (3H, m), 4.73-4.81 (1H, m), 5.05 (1H, dd), 5.24-5.32 (1H, m), 6.37 (1H, d), 6.47-6.53 (1H, m), 6.72 (1H, t), 7.17-7.23 (1H, m), 7.28 (1H, dd), 7.46 (1H, d), 7.55 (1H, t), 7.94 (1H, d).

### Example 3b

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid, Isomer 2

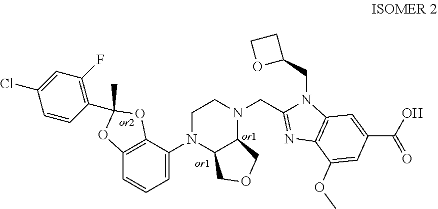 [SMILES: COc1cc(C(=O)O)cc2c1nc(CN1CCN(c3cccc4c3O[C@@](C)(c3ccc(Cl)cc3F)O4)[C@@H]3COC[C@@H]31)n2C[C@@H]1CCO1]

A mixture of methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 2 Intermediate 31 (48 mg, 0.07 mmol) and 1,3,4,6,7,8-hexahydro-2H-pyrimido[1,2-a]pyrimidine (20 mg, 0.14 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, dissolved in DMSO, filtered and purified by preparative HPLC, PrepMethod A, (gradient: 20-90%) to give the title compound (35 mg, 75%); HRMS (ESI) m/z [M+H]⁺ calcd for C₃₄H₃₅ClFN₄O₇: 665.2172, found: 665.2174; ¹H NMR (500 MHz, CD₃OD) 2.02 (3H, s), 2.31-2.42 (1H, m), 2.44-2.53 (1H, m), 2.65-2.72 (1H, m), 2.72-2.79 (1H, m), 3.02-3.08 (1H, m), 3.13-3.27 (2H, m), 3.61 (1H, d), 3.75 (1H, t), 3.82-3.92 (2H, m), 4.04 (3H, s), 4.13-4.21 (1H, m), 4.24-4.3 (1H, m), 4.53-4.59 (3H, m), 4.59-4.66 (1H, m), 5.06 (1H, dd), 5.25-5.33 (1H, m), 6.37-6.43 (1H, m), 6.43-6.49 (1H, m), 6.71 (1H, t), 7.15-7.21 (1H, m), 7.22-7.28 (1H, m), 7.46 (1H, d), 7.56 (1H, t), 7.94 (1H, d).

### Example 3c

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid, Isomer 4

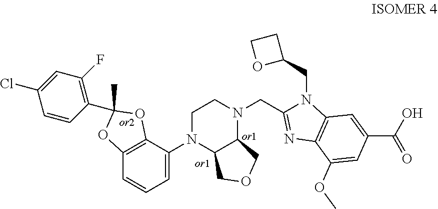 [SMILES: COc1cc(C(=O)O)cc2c1nc(CN1CCN(c3cccc4c3O[C@@](C)(c3ccc(Cl)cc3F)O4)[C@@H]3COC[C@@H]31)n2C[C@@H]1CCO1]

A mixture of methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylate, Isomer 4 Intermediate 32 (40 mg, 0.06 mmol) and 1,3,4,6,7,8-hexahydro-2H-pyrimido[1,2-a]pyrimidine (16 mg, 0.12 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, dissolved in DMSO, filtered and purified by preparative HPLC, PrepMethod A, (gradient: 20-90%) to give the title compound (27 mg, 69%); HRMS (ESI) m/z [M+H]⁺ calcd for C₃₄H₃₅ClFN₄O₇: 665.2172, found: 665.2192; ¹H NMR (500 MHz, CD₃OD) 2.03 (3H, s), 2.45-2.54 (1H, m), 2.59-2.66 (1H, m), 2.7-2.76 (1H, m), 2.75-2.84 (1H, m), 3.03-3.08 (1H, m), 3.17-3.3 (2H, m), 3.55 (1H, d), 3.81 (1H, t), 3.92-4 (2H, m), 4.03 (3H, s), 4.41-4.44 (1H, m), 4.49 (1H, dd), 4.54 (1H, d), 4.57-4.64 (1H, m), 4.64-4.75 (2H, m), 5.17-5.26 (2H, m), 6.43 (1H, dd), 6.47 (1H, dd), 6.73 (1H, t), 7.16-7.22 (1H, m), 7.26 (1H, dd), 7.44 (1H, d), 7.58 (1H, t), 7.94 (1H, d).

### Example 4a

### 4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid, Isomer 3

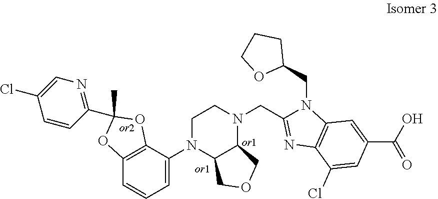 [SMILES: C[C@]1(c2ccc(Cl)cn2)Oc2cccc(N3CCN(Cc4nc5c(Cl)cc(C(=O)O)cc5n4C[C@@H]4CCCO4)[C@H]4COC[C@H]43)c2O1]

A mixture of methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate, Isomer 3 Intermediate 34 (50 mg, 0.07 mmol) and 1,3,4,6,7,8-hexahydro-2H-pyrimido[1,2-a]pyrimidine (20 mg, 0.15 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, dissolved in DMSO, filtered and purified by preparative HPLC, PrepMethod A, (gradient: 20-90%) to give the title compound (52 mg, 105%); HRMS (ESI) m/z [M+H]⁺ calcd for C₃₃H₃₄Cl₂N₅O₆: 666.1880, found: 666.1908; ¹H NMR (500 MHz, CDCl₃) 1.49-1.6 (1H, m), 1.71-1.82 (1H, m), 1.82-1.94 (1H, m), 2.01 (2H, s), 2.07 (5H, s), 2.57-3.46 (5H, m), 3.54-3.67 (1H, m), 3.69-3.74 (1H, m), 3.77-3.88 (2H, m), 3.91-4.04 (1H, m), 4.17-4.25 (1H, m), 4.29-4.36 (1H, m), 4.47-4.59 (1H, m), 4.65-4.76 (1H, m), 4.94-5.09 (1H, m), 6.35 (1H, d), 6.53 (1H, d), 6.74 (1H, t), 7.55 (1H, d), 7.69 (1H, dd), 7.87-8.13 (2H, m), 8.58-8.62 (1H, m).

### Example 4b

### 4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid, Isomer 4

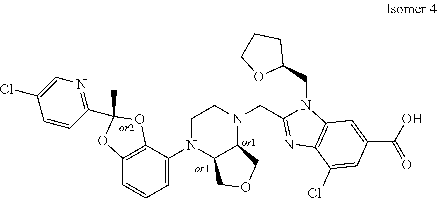 [SMILES: C[C@]1(c2ccc(Cl)cn2)Oc2cccc(N3CCN(Cc4nc5c(Cl)cc(C(=O)O)cc5n4C[C@@H]4CCCO4)[C@H]4COC[C@H]43)c2O1]

A mixture of methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate, Isomer 4 Intermediate 35 (50 mg, 0.07 mmol) and 1,3,4,6,7,8-hexahydro-2H-pyrimido[1,2-a]pyrimidine (20 mg, 0.15 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, dissolved in DMSO, filtered and purified by preparative HPLC, PrepMethod A, (gradient: 20-90%) to give the title compound (32 mg, 66%); HRMS (ESI) m/z [M+H]⁺ calcd for C₃₃H₃₄Cl₂N₅O₆: 666.1886, found: 666.1913; 1H NMR (500 MHz, CD₃OD) 1.57-1.68 (1H, m), 1.74-1.94 (2H, m), 2.02 (3H, s), 2.09-2.19 (1H, m), 2.48-2.56 (1H, m), 2.80 (1H, dt), 3.11-3.16 (1H, m), 3.16-3.27 (2H, m), 3.69-3.79 (3H, m), 3.8-3.94 (3H, m), 4.27-4.38 (2H, m), 4.39-4.53 (2H, m), 4.67 (1H, td), 5.05 (1H, dd), 6.44 (1H, dd), 6.49 (1H, dd), 6.75 (1H, t), 7.64-7.7 (1H, m), 7.88 (1H, dd), 7.94 (1H, d), 8.25 (1H, d), 8.60 (1H, dd).

### Example 5a

### 2-((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid, Isomer 1

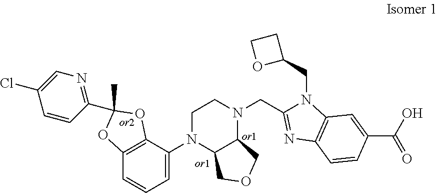 [SMILES: C[C@]1(c2ccc(Cl)cn2)Oc2cccc(N3CCN(Cc4nc5ccc(C(=O)O)cc5n4C[C@@H]4CCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 1 Intermediate 37 (34 mg, 53 mmol) and LiOH·H₂O (2.5 eq) in THF:H₂O (v/v, 1:3, 4 mL) was stirred at 20° C. overnight. The reaction mixture was concentrated under reduced pressure and acidified with citric acid. The product was collected by filtration, washed with water (3×75 mL), and then dried in vacuo at 60° C. to give the title compound (25 mg); HRMS (ESI) m/z [M+H]⁺ calcd for C₃₂H₃₃ClN₅O₆: 618.2119, found: 618.2138; 1H NMR (500 MHz, DMSO-d₆) δ 12.73 (s, 1H), 8.68 (s, 1H), 8.24 (s, 1H), 8.00 (d, 1H), 7.79 (d, 1H), 7.61 (t, 2H), 6.72 (t, 1H), 6.51 (d, 1H), 6.40 (d, 1H), 5.14-4.80 (m, 2H), 4.68-4.35 (m, 5H), 4.23 (d, 1H), 3.85-3.55 (m, 2H), 3.50-3.33 (m, 3H), 3.22-2.97 (m, 3H), 2.67 (d, 3H), 2.01 (s, 3H).

### Example 5b

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid, Isomer 2

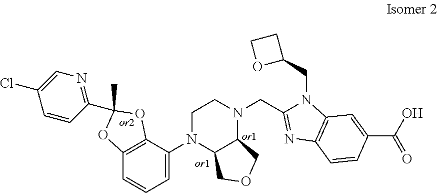 [SMILES: C[C@]1(c2ccc(Cl)cn2)Oc2cccc(N3CCN(Cc4nc5ccc(C(=O)O)cc5n4C[C@@H]4CCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 2 Intermediate 38 (33 mg, 52 mmol) and LiOH·H₂O (2.5 eq) in THF:H₂O (v/v, 1:3, 4 mL) was stirred at 20° C. overnight. The reaction mixture was concentrated under reduced pressure and acidified with citric acid. The product was collected by filtration, washed with water (3×75 mL), and then dried in vacuo at 60° C. to give the title compound (24 mg); HRMS (ESI) m/z [M+H]⁺ calcd for C₃₂H₃₃ClN₅O₆: 618.2119, found: 618.2157; 1H NMR (500 MHz, DMSO-d6) δ 12.5 (brs, 1H) 8.67 (d, 1H), 8.25 (s, 1H), 7.99 (dd, 1H), 7.80 (d, 1H), 7.62 (dd, 2H), 6.72 (t, 1H), 6.51 (d, 1H), 6.39 (d, 1H), 5.13 (d, 1H), 4.81 (d, 1H), 4.66 (m, 1H), 4.51 (q, 1H), 4.47-4.33 (m, 2H), 4.22-4.11 (m, 1H), 4.05 (d, 1H), 3.73 (dd, 1H), 3.60 (dd, 2H), 3.33 (d, 2H), 3.13-3.02 (m, 3H), 2.73 (d, 1H), 2.60 (d, 1H), 2.26 (t, 1H), 2.00 (s, 3H).

### Example 5c

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid, Isomer 3

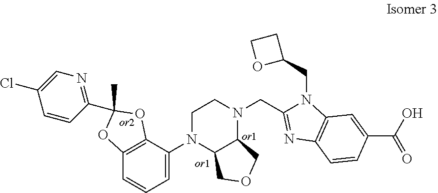 [SMILES: C[C@]1(c2ccc(Cl)cn2)Oc2cccc(N3CCN(Cc4nc5ccc(C(=O)O)cc5n4C[C@@H]4CCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 3 Intermediate 39 (32 mg, 51 mmol) and LiOH·H₂O (2.5 eq) in in THF:H₂O (v/v, 1:3, 4 mL) was stirred at 20° C. overnight. The reaction mixture was concentrated under reduced pressure and acidified with citric acid. The product was collected by filtration, washed with water (3×75 mL), and then dried in vacuo at 60° C. to give the title compound (27 mg); HRMS (ESI) m/z [M+H]⁺ calcd for C₃₂H₃₃ClN₅O₆: 618.2119, found: 618.2151; 1H NMR (500 MHz, DMSO-d6) δ 12.65 (brs, 1H), 8.70 (d, 1H), 8.25 (d, 1H), 7.99 (dd, 1H), 7.79 (dd, 1H), 7.62 (t, 2H), 6.73 (t, 1H), 6.50 (d, 1H), 6.42 (d, 1H), 5.04 (q, 1H), 4.95 (dd, 1H), 4.51 (m, 5H), 4.26 (d, 1H), 3.83 (dd, 1H), 3.77 (t, 1H), 3.70 (t, 1H), 3.45 (d, 1H), 3.25 (d, 1H), 3.09 (t, 1H), 3.00 (d, 1H), 2.68 (m, 2H), 2.42-2.30 (m, 2H), 1.99 (s, 3H).

### Example 5d

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid, Isomer 4

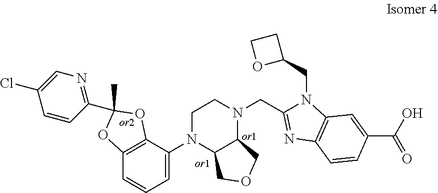 [SMILES: C[C@]1(c2ccc(Cl)cn2)Oc2cccc(N3CCN(Cc4nc5ccc(C(=O)O)cc5n4C[C@@H]4CCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 4 Intermediate 40 (32 mg, 51 mmol) and LiOH·H₂O (2.5 eq) was stirred in THF:H₂O (v/v, 1:3, 4 mL) at 20° C. overnight. The reaction mixture was concentrated under reduced pressure and acidified with citric acid. The product was collected by filtration, washed with water (3×75 mL), and then dried in vacuo at 60° C. to give the title compound (24 mg); HRMS (ESI) m/z [M+H]⁺ calcd for C₃₂H₃₃ClN₅O₆: 618.2119, found: 618.2153; 1H NMR (500 MHz, DMSO-d6) δ 12.64 (brs, 1H) 8.70 (d, 1H), 8.25 (d, 1H), 7.99 (dd, 1H), 7.80 (dd, 1H), 7.63 (dd, 2H), 6.72 (t, 1H), 6.45 (dd, 2H), 5.15 (d, 1H), 4.83 (dd, 1H), 4.66 (dd, 1H), 4.61-4.31 (m, 3H), 4.23-3.98 (m, 2H), 3.85-3.59 (m, 4H), 3.21 (d, 1H), 3.15-2.95 (m, 2H), 2.78-2.61 (m, 3H), 2.26 (m, 1H), 1.98 (s, 3H).

### Example 6a

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid, Isomer 1

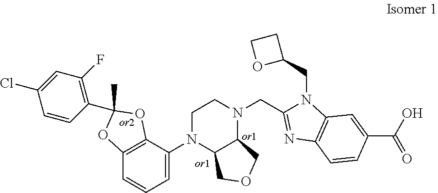 [SMILES: C[C@]1(c2ccc(Cl)cc2F)Oc2cccc(N3CCN(Cc4nc5ccc(C(=O)O)cc5n4C[C@@H]4CCO4)[C@H]4COC[C@H]43)c2O1]

LiOH·H₂O (2 eq) was added to a solution of methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 1 Intermediate 42 (38 mg, 0.059 mmol) in THF-H₂O (2:1, 5 mL), and the reaction mixture was stirred at rt for 16 h. The mixture was acidified by NaH₂PO₄ (aq) and diluted with water. The precipitate formed was collected by filtration, washed with water and dried to afford the title compound (9.6 mg); HRMS (ESI) m/z [M+H]⁺ calcd for C₃₃H₃₃ClFN₄O₆: 635.2068, found: 635.2084; 1H NMR (600 MHz, DMSO-d6) δ 12.76 (s, 1H), 8.25 (d, 1H), 7.80 (dd, 1H), 7.65 (d, 1H), 7.59-7.48 (m, 2H), 7.31 (dd, 1H), 6.72 (t, 1H), 6.53 (d, 1H), 6.37 (d, 1H), 5.13 (m, 1H), 4.81 (dd, 1H), 4.65 (dd, 1H), 4.56 (td, 1H), 4.49-4.34 (m, 2H), 4.16 (dt, 1H), 4.08 (dd, 1H), 3.74 (dd, 1H), 3.66-3.52 (m, 2H), 3.17-3.01 (m, 3H), 2.73 (dt, 1H), 2.68-2.55 (m, 2H), 2.44-2.32 (m, 1H), 2.26 (dq, 1H), 2.02 (s, 3H).

### Example 6b

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid, Isomer 2

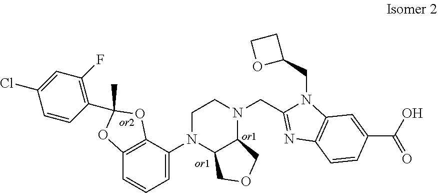 [SMILES: C[C@]1(c2ccc(Cl)cc2F)Oc2cccc(N3CCN(Cc4nc5ccc(C(=O)O)cc5n4C[C@@H]4CCO4)[C@H]4COC[C@H]43)c2O1]

LiOH·H₂O (2 eq) was added to a solution of methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 2 Intermediate 43 (27 mg, 0.042 mmol) in a mixture of THF-H₂O (2:1, 5 mL), and the reaction mixture was stirred at rt for 16 h. The mixture was acidified by NaH₂PO₄ (aq) and diluted with water. The precipitate formed was collected by filtration, washed with water and dried to afford the title compound (5 mg); HRMS (ESI) m/z [M+H]⁺ calcd for C₃₃H₃₃ClFN₄O₆: 635.2068, found: 635.2064; 1H NMR (600 MHz, DMSO-d6) δ 12.77 (s, 1H), 8.25 (d, 1H), 7.80 (dd, 1H), 7.66 (d, 1H), 7.62-7.47 (m, 2H), 7.32 (dd, 1H), 6.71 (t, 1H), 6.49 (d, 1H), 6.40 (d, 1H), 5.26-5.04 (m, 1H), 4.84 (dd, 1H), 4.66 (dd, 1H), 4.50 (td, 1H), 4.44 (td, 2H), 4.24-4.02 (m, 2H), 3.82-3.65 (m, 3H), 3.57 (d, 1H), 3.16-2.95 (m, 2H), 2.83-2.64 (m, 1H), 2.45-2.32 (m, 3H), 2.26 (tt, 1H), 1.98 (s, 3H).

### Example 6c

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid, Isomer 3

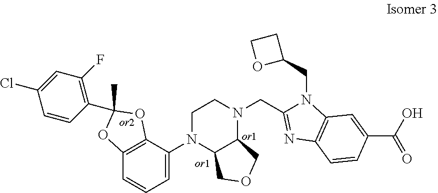 [SMILES: C[C@]1(c2ccc(Cl)cc2F)Oc2cccc(N3CCN(Cc4nc5ccc(C(=O)O)cc5n4C[C@@H]4CCO4)[C@H]4COC[C@H]43)c2O1]

LiOH·H₂O (2 eq) was added to a solution of methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 3 Intermediate 44 (29 mg, 0.044 mmol) in THF-H₂O (2:1, 5 mL), and the reaction mixture was stirred at rt for 16 h. The mixture was acidified by NaH₂PO₄ (aq) and diluted with water. The precipitate formed was collected by filtration, washed with water and dried to afford the title compound (5 mg); HRMS (ESI) m/z [M+H]⁺ calcd for C₃₃H₃₃ClFN₄O₆: 635.2068, found: 635.2074; 1H NMR (600 MHz, DMSO-d6) δ 12.75 (brs, 1H), 8.24 (d, 1H), 7.79 (dd, 1H), 7.69-7.60 (m, 1H), 7.61-7.48 (m, 2H), 7.32 (dd, 1H), 6.72 (t, 1H), 6.54 (d, 1H), 6.39 (d, 1H), 5.02 (q, 1H), 4.94 (dd, 1H), 4.65 (td, 1H), 4.55-4.49 (m, 2H), 4.49-4.40 (m, 1H), 4.26 (d, 1H), 3.81 (dd, 1H), 3.78-3.55 (m, 2H), 3.46 (d, 1H), 3.21-3.11 (m, 1H), 3.12-2.96 (m, 3H), 2.76-2.63 (m, 2H), 2.41-2.31 (m, 2H), 2.02 (s, 3H).

### Example 6d

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid, Isomer 4

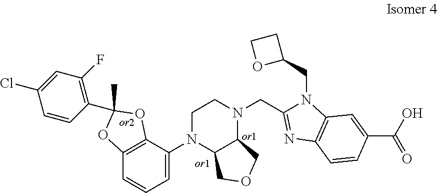 [SMILES: C[C@]1(c2ccc(Cl)cc2F)Oc2cccc(N3CCN(Cc4nc5ccc(C(=O)O)cc5n4C[C@@H]4CCO4)[C@H]4COC[C@H]43)c2O1]

LiOH·H₂O (2 eq) was added to a solution of methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 4 Intermediate 45 (25 mg, 0.038 mmol) in THF-H₂O (2:1, 5 mL), and the reaction mixture was stirred at rt for 16 h. The mixture was acidified by NaH₂PO₄ (aq) and diluted with water. The precipitate formed was collected by filtration, washed with water and dried to afford the title compound (5 mg); HRMS (ESI) m/z [M+H]⁺ calcd for C₃₃H₃₃ClFN₄O₆: 635.2068, found: 635.2066; 1H NMR (600 MHz, DMSO-d6) δ 12.75 (brs, 1H), 8.25 (d, 1H), 7.79 (dd, 1H), 7.70-7.56 (m, 1H), 7.56-7.47 (m, 2H), 7.32 (dd, 1H), 6.72 (t, 1H), 6.46 (dd, 2H), 5.16-4.90 (m, 2H), 4.66-4.38 (m, 5H), 4.28 (d, 1H), 3.93-3.69 (m, 3H), 3.46 (d, 1H), 3.19-2.94 (m, 2H), 2.79-2.64 (m, 3H), 2.44-2.29 (m, 2H), 1.99 (s, 3H).

### Example 7a

### rel-1-(2-(1H-Pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid, Isomer 1

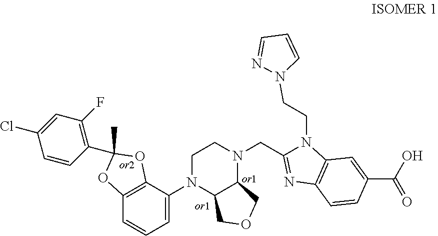 [SMILES: C[C@]1(c2ccc(Cl)cc2F)Oc2cccc(N3CCN(Cc4nc5ccc(C(=O)O)cc5n4CCn4cccn4)[C@H]4COC[C@H]43)c2O1]

A solution of rel-methyl 1-(2-(1H-pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate Isomer 1 Intermediate 47 (129 mg, 0.19 mmol) and 1,3,4,6,7,8-hexahydro-2H-pyrimido[1,2-a]pyrimidine (59 mg, 0.42 mmol) in a mixture of MeCN (1 mL) and water (0.2 mL) was stirred at rt overnight. The reaction mixture was concentrated and the residue was purified by preparative HPLC, PrepMethod E (gradient: 5-95%) to give the title compound (34 mg, 27%); HRMS (ESI) m/z [M+H]⁺ calcd for C₃₄H₃₃ClFN₆O₅: 659.2180, found: 659.2178; 1H NMR (600 MHz, DMSO-d₆) 2.02 (3H, s), 2.35-2.38 (1H, m), 2.71-2.75 (1H, m), 3.02-3.05 (1H, m), 3.09-3.15 (2H, m), 3.29-3.35 (2H, m), 3.60 (1H, t), 3.74-3.84 (2H, m), 4.16 (1H, d), 4.53-4.62 (3H, m), 4.72-4.85 (2H, m), 6.06-6.18 (1H, m), 6.38 (1H, d), 6.54 (1H, d), 6.72 (1H, t), 7.26-7.37 (2H, m), 7.44 (1H, s), 7.48-7.59 (2H, m), 7.62 (1H, d), 7.79 (1H, d), 7.90 (1H, s).v

### Example 7b

### rel-1-(2-(1H-Pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid, Isomer 2

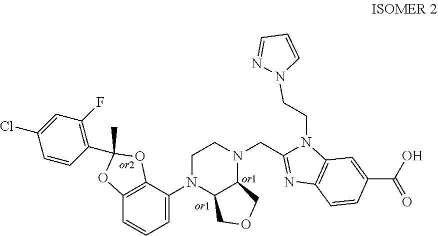 [SMILES: C[C@]1(c2ccc(Cl)cc2F)Oc2cccc(N3CCN(Cc4nc5ccc(C(=O)O)cc5n4CCn4cccn4)[C@H]4COC[C@H]43)c2O1]

A solution of rel-methyl 1-(2-(1H-pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate, Isomer 2 Intermediate 48 (33 mg, 0.05 mmol) and 1,3,4,6,7,8-hexahydro-2H-pyrimido[1,2-a]pyrimidine (19 mg, 0.14 mmol) in a mixture of MeCN (1 mL) and water (0.2 mL) was stirred at rt overnight. The reaction mixture was concentrated, and the residue was dissolved in DMSO and purified by preparative HPLC, PrepMethod A, (gradient: 40-80%) to give the title compound (22 mg, 68%) as a white powder; HRMS (ESI) m/z [M+H]⁺ calcd for C₃₄H₃₃ClFN₆O₅: 659.2180, found: 659.2198; 1H NMR (500 MHz, DMSO) 2.00 (3H, s), 2.34-2.44 (1H, m), 2.67-2.77 (1H, m), 2.98-3.03 (1H, m), 3.12 (1H, t), 3.29-3.33 (2H, m), 3.69-3.76 (2H, m), 3.76-3.81 (1H, m), 3.84 (1H, d), 4.18 (1H, d), 4.51 (1H, td), 4.61 (2H, t), 4.73-4.92 (2H, m), 6.11 (1H, t), 6.42 (1H, d), 6.51 (1H, d), 6.73 (1H, t), 7.33 (1H, dd), 7.35 (1H, d), 7.44 (1H, d), 7.51-7.59 (2H, m), 7.63 (1H, d), 7.78 (1H, dd), 7.91 (1H, d), 12.74 (1H, s).

### Example 7c

### rel-1-(2-(1H-Pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid, Isomer 3

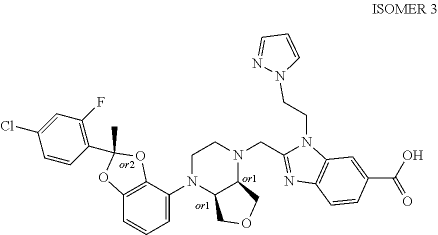 [SMILES: C[C@]1(c2ccc(Cl)cc2F)Oc2cccc(N3CCN(Cc4nc5ccc(C(=O)O)cc5n4CCn4cccn4)[C@H]4COC[C@H]43)c2O1]

A solution of rel-methyl 1-(2-(1H-pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylate, Isomer 3 Intermediate 49 (16 mg, 0.02 mmol) and 1,3,4,6,7,8-hexahydro-2H-pyrimido[1,2-a]pyrimidine (10 mg, 0.07 mmol) in a mixture of MeCN (1 mL) and water (0.2 mL) was stirred at rt overnight. The reaction mixture was concentrated and the residue was dissolved in DMSO and purified by preparative HPLC, PrepMethod A, (gradient: 40-80%) to give the title compound (0.011 g, 70%) as a white powder; HRMS (ESI) m/z [M+H]⁺ calcd for C₃₄H₃₃ClFN₆O₅: 659.2180, found: 659.2188; 1H NMR (500 MHz, DMSO) 2.00 (3H, s), 2.33-2.44 (1H, m), 2.69-2.79 (1H, m), 3.01 (1H, t), 3.05-3.17 (1H, m), 3.3-3.32 (2H, m), 3.69-3.76 (2H, m), 3.79 (1H, dd), 3.84 (1H, d), 4.18 (1H, d), 4.51 (1H, td), 4.61 (2H, t), 4.7-4.88 (2H, m), 6.11 (1H, t), 6.42 (1H, d), 6.51 (1H, d), 6.73 (1H, t), 7.33 (1H, dd), 7.35 (1H, d), 7.44 (1H, d), 7.49-7.59 (2H, m), 7.62 (1H, d), 7.78 (1H, dd), 7.91 (1H, d), 12.74 (1H, s).

### Example 8a

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid, Isomer 3

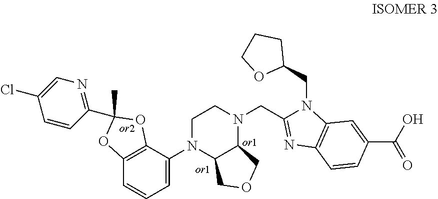 [SMILES: C[C@]1(c2ccc(Cl)cn2)Oc2cccc(N3CCN(Cc4nc5ccc(C(=O)O)cc5n4C[C@@H]4CCCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate, Isomer 3 Intermediate 51 (94 mg, 0.15 mmol) and 1,3,4,6,7,8-hexahydro-2H-pyrimido[1,2-a]pyrimidine (40 mg, 0.29 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, and the residue was dissolved in DMSO, filtered and purified by preparative HPLC, PrepMethod F, (gradient: 20-70%), to give the title compound (54 mg, 58%); HRMS (ESI) m/z [M+H]⁺ calcd for C₃₃H₃₅ClN₅O₆: 632.2270, found: 632.2284; 1H NMR (600 MHz, DMSO-d₆) 1.48-1.58 (1H, m), 1.7-1.81 (2H, m), 1.96-2.06 (4H, m), 2.5-2.51 (1H, m), 2.69-2.77 (1H, m), 3.10 (3H, q), 3.37 (1H, t), 3.55-3.66 (3H, m), 3.69-3.81 (2H, m), 4.11 (1H, dd), 4.19-4.27 (1H, m), 4.27-4.42 (2H, m), 4.48-4.56 (1H, m), 4.76 (1H, dd), 6.40 (1H, dd), 6.53 (1H, dd), 6.7-6.76 (1H, m), 7.60 (1H, dd), 7.62-7.66 (1H, m), 7.80 (1H, dd), 7.99 (1H, dd), 8.17-8.22 (1H, m), 8.68 (1H, dd).

### Example 8b

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid, Isomer 4

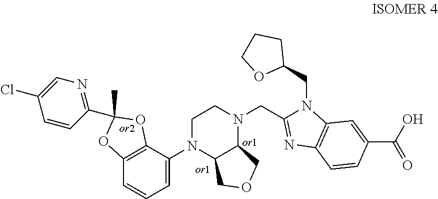 [SMILES: C[C@]1(c2ccc(Cl)cn2)Oc2cccc(N3CCN(Cc4nc5ccc(C(=O)O)cc5n4C[C@@H]4CCCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylate, Isomer 4 Intermediate 52 (60 mg, 0.09 mmol) and 1,3,4,6,7,8-hexahydro-2H-pyrimido[1,2-a]pyrimidine (26 mg, 0.19 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, and the residue was dissolved in DMSO, filtered and purified by preparative HPLC, PrepMethod F, (gradient: 20-70%), to give the title compound (39 mg, 67%); HRMS (ESI) m/z [M+H]⁺ calcd for C₃₃H₃₅ClN₅O₆: 632.2270, found: 632.2292; 1H NMR (600 MHz, DMSO-d₆) 1.54 (1H, dq), 1.68-1.81 (2H, m), 1.97-2.06 (4H, m), 2.39-2.47 (2H, m), 2.7-2.78 (1H, m), 3.04-3.13 (2H, m), 3.21 (1H, dt), 3.58-3.65 (2H, m), 3.68 (1H, t), 3.71-3.77 (2H, m), 3.80 (1H, dd), 4.1-4.16 (1H, m), 4.21-4.29 (1H, m), 4.35-4.44 (2H, m), 4.5-4.56 (1H, m), 4.79 (1H, dd), 6.42 (1H, dd), 6.52 (1H, dd), 6.74 (1H, t), 7.58-7.67 (2H, m), 7.80 (1H, dd), 7.99 (1H, dd), 8.21-8.25 (1H, m), 8.7-8.73 (1H, m).

### Example 9a

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid, Isomer 1

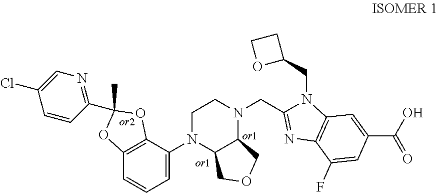 [SMILES: C[C@]1(c2ccc(Cl)cn2)Oc2cccc(N3CCN(Cc4nc5c(F)cc(C(=O)O)cc5n4C[C@@H]4CCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 1 Intermediate 54 and LiOH·H₂O (3 eq) in THF:H₂O (1:3, 4 mL) was stirred at 20° C. overnight. The reaction mixture was concentrated under reduced pressure and acidified with citric acid. The product was collected by filtration, washed with water (3×75 mL), and then dried in vacuo at 60° C. to give the title compound (28 mg); HRMS (ESI) m/z [M+H]⁺ calcd for C₃₂H₃₂ClFN₅O₆: 636.2025, found: 636.2036; 1H NMR (600 MHz, DMSO-d6) δ 13.05 (s, 1H), 8.67 (d, 1H), 8.14 (d, 1H), 7.99 (dd, 1H), 7.59 (d, 1H), 7.51 (dd, 1H), 6.72 (t, 1H), 6.51 (d, 1H), 6.39 (d, 1H), 5.18-5.07 (m, 1H), 4.84 (dd, 1H), 4.69 (dd, 1H), 4.52 (m, 1H), 4.48-4.35 (m, 2H), 4.17 (dt, 1H), 4.05 (dd, 1H), 3.73 (dd, 1H), 3.66-3.55 (m, 2H), 3.35 (t, 1H), 3.14-3.03 (m, 3H), 2.72 (dt, 1H), 2.66-2.56 (m, 1H), 2.45-2.33 (m, 1H), 2.29-2.18 (m, 1H), 2.00 (s, 3H).

### Example 9b

### 2-((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid, Isomer 2

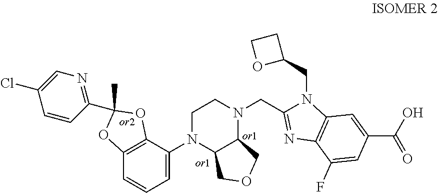 [SMILES: C[C@]1(c2ccc(Cl)cn2)Oc2cccc(N3CCN(Cc4nc5c(F)cc(C(=O)O)cc5n4C[C@@H]4CCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 2 Intermediate 55 and LiOH·H₂O (3 eq) in THF:H₂O (1:3, 4 mL) was stirred at 20° C. overnight. The reaction mixture was concentrated under reduced pressure and acidified with citric acid. The product was collected by filtration, washed with water (3×75 mL), and then dried in vacuo at 60° C. to give the title compound (21 mg); HRMS (ESI) m/z [M+H]⁺ calcd for C₃₂H₃₂ClFN₅O₆: 636.2025, found: 636.2036; 1H NMR (600 MHz, DMSO-d6) δ 13.07 (s, 1H), 8.70 (d, 1H), 8.14 (d, 1H), 7.99 (dd, 1H), 7.61 (d, 1H), 7.51 (dd, 1H), 6.73 (t, 1H), 6.50 (d, 1H), 6.41 (d, 1H), 5.20-5.10 (m, 1H), 4.87 (dd, 1H), 4.70 (dd, 1H), 4.52 (td, 1H), 4.44 (dt, 2H), 4.16 (dt, 1H), 4.07 (d, 1H), 3.76 (dd, 1H), 3.72 (t, 1H), 3.66 (t, 1H), 3.59 (d, 1H), 3.22 (dt, 1H), 3.12-3.01 (m, 1H), 2.75-2.68 (m, 2H), 2.66-2.57 (m, 1H), 2.45-2.34 (m, 1H), 2.25 (m, 1H), 1.98 (s, 3H).

### Example 9c

### 2-((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid, Isomer 3

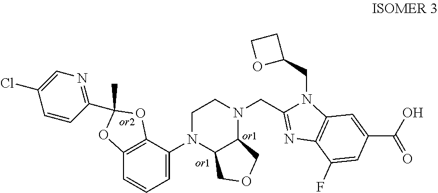 [SMILES: C[C@]1(c2ccc(Cl)cn2)Oc2cccc(N3CCN(Cc4nc5c(F)cc(C(=O)O)cc5n4C[C@@H]4CCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 3 Intermediate 56 and LiOH·H₂O (3 eq) in THF:H₂O (1:3, 4 mL) was stirred at 20° C. overnight. The reaction mixture was concentrated under reduced pressure and acidified with citric acid. The product was collected by filtration, washed with water (3×75 mL), and then dried in vacuo at 60° C. to give the title compound (35 mg); HRMS (ESI) m/z [M+H]⁺ calcd for C₃₂H₃₂ClFN₅O₆: 636.2025, found: 636.2064; 1H NMR (600 MHz, DMSO-d₆) δ 13.05 (s, 1H), 8.68 (d, 1H), 8.14 (s, 1H), 8.01 (dd, 1H), 7.60 (d, 1H), 7.49 (d, 1H), 6.73 (t, 1H), 6.51 (d, 1H), 6.41 (d, 1H), 5.07-5.00 (m, 1H), 4.97 (dd, 1H), 4.61 (td, 1H), 4.58-4.49 (m, 2H), 4.49-4.39 (m, 2H), 4.23 (d, 1H), 3.80 (dd, 1H), 3.65 (t, 1H), 3.49 (d, 1H), 3.37 (t, 1H), 3.19-3.12 (m, 1H), 3.13-3.05 (m, 1H), 3.03 (d, 1H), 2.73-2.63 (m, 2H), 2.44-2.37 (m, 2H), 2.01 (s, 3H).

### Example 9d

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid, Isomer 4

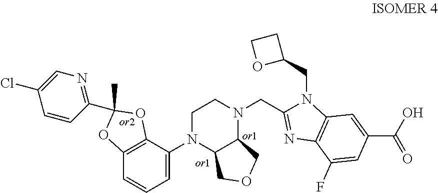 [SMILES: C[C@]1(c2ccc(Cl)cn2)Oc2cccc(N3CCN(Cc4nc5c(F)cc(C(=O)O)cc5n4C[C@@H]4CCO4)[C@H]4COC[C@H]43)c2O1]

A mixture of methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 4 Intermediate 57 and LiOH·H₂O (3 eq) in THF:H₂O (1:3, 4 mL) was stirred at 20° C. overnight. The reaction mixture was concentrated under reduced pressure and acidified with citric acid. The product was collected by filtration, washed with water (3×75 mL), and then dried in vacuo at 60° C. to give the title compound (23 mg); HRMS (ESI) m/z [M+H]⁺ calcd for C₃₂H₃₂ClFN₅O₆: 636.2025, found: 636.2051; 1H NMR (600 MHz, DMSO-d₆) δ 13.06 (s, 1H), 8.70 (d, 1H), 8.14 (d, 1H), 7.99 (dd, 1H), 7.61 (d, 1H), 7.50 (dd, 1H), 6.73 (t, 1H), 6.51 (d, 1H), 6.43 (d, 1H), 5.10-4.90 (m, 2H), 4.63-4.41 (m, 5H), 4.26 (d, 1H), 3.82 (dd, 1H), 3.77 (t, 1H), 3.71 (t, 1H), 3.48 (d, 1H), 3.28-3.23 (m, 2H), 3.09 (t, 1H), 3.00 (t, 1H), 2.68 (m, 2H), 2.44-2.37 (m, 1H), 1.99 (s, 3H).

### Example 10

### rel-1-(2-(1H-Pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid Isomer 1

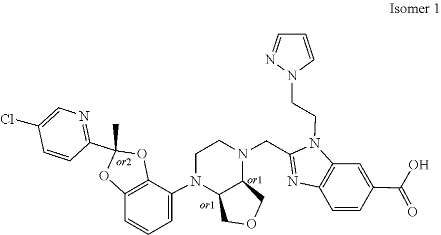 [SMILES: C[C@]1(c2ccc(Cl)cn2)Oc2cccc(N3CCN(Cc4nc5ccc(C(=O)O)cc5n4CCn4cccn4)[C@H]4COC[C@H]43)c2O1]

A solution of rel-methyl 1-(2-(1H-pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylate, Isomer 1 Intermediate 124 (131 mg, 0.20 mmol) and 1,3,4,6,7,8-hexahydro-2H-pyrimido[1,2-a]pyrimidine (60 mg, 0.43 mmol) in a mixture of MeCN (1 mL) and water (0.2 mL) was stirred at rt overnight. The reaction mixture was concentrated and the crude compound was purified by preparative HPLC, PrepMethod A, (gradient: 35-75%) to give the title compound Isomer 1 (97 mg, 76%) as a white powder; HRMS (ESI) m/z [M+H]⁺ calcd for C₃₃H₃₃ClN₇O₅: 642.2226, found: 642.2230; ¹H NMR (500 MHz, CD₃OD) 2.03 (s, 3H), 2.44-2.52 (m, 1H), 2.79-2.87 (m, 1H), 3.03-3.11 (m, 1H), 3.13-3.20 (m, 1H), 3.20-3.28 (m, 1H), 3.37-3.46 (m, 2H), 3.73 (t, 1H), 3.84-3.96 (m, 2H), 4.21 (dd, 1H), 4.63-4.75 (m, 3H), 4.78 (dt, 1H), 4.97 (ddd, 1H), 6.11 (s, 1H), 6.42 (d, 1H), 6.51 (d, 1H), 6.74 (t, 1H), 7.15 (d, 1H), 7.50 (d, 1H), 7.64 (dd, 2H), 7.84-7.90 (m, 2H), 7.93 (dd, 1H), 8.60 (d, 1H).

### Example 11

### rel-1-(2-(1H-Pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylic acid

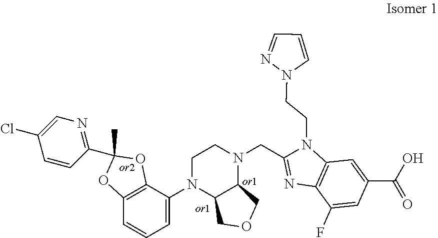 [SMILES: C[C@]1(c2ccc(Cl)cn2)Oc2cccc(N3CCN(Cc4nc5c(F)cc(C(=O)O)cc5n4CCn4cccn4)[C@H]4COC[C@H]43)c2O1]

A solution of rel-methyl 1-(2-(1H-pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylate, Isomer 1 Intermediate 125 (92 mg, 0.14 mmol) and 1,3,4,6,7,8-hexahydro-2H-pyrimido[1,2-a]pyrimidine (42 mg, 0.30 mmol) in a mixture of MeCN (1 mL) and water (0.2 mL) was stirred at rt overnight. The reaction mixture was concentrated under reduced pressure and the residue was partitioned between DCM (2 mL) and 1 M HCl (aq, 2 mL). The aqueous phase was extracted with another portion of DCM (2 mL) and the combined organic layer was concentrated and dried under reduced pressure to give the title compound Isomer 1 (87 mg, 97%) as a white solid; HRMS (ESI) m/z [M+H]⁺ calcd for C₃₃H₃₂ClFN₇O₅: 660.2132, found: 660.2160; ¹H NMR (500 MHz, CD₃OD) 2.02 (s, 3H), 2.75-2.88 (m, 1H), 3.07-3.17 (m, 1H), 3.24-3.29 (m, 1H), 3.32-3.39 (m, 1H), 3.39-3.46 (m, 1H), 3.49 (t, 1H), 3.7-3.79 (m, 2H), 3.95 (dd, 1H), 4.06 (d, 1H), 4.23 (dd, 1H), 4.65-4.78 (m, 3H), 4.79-4.85 (m, 1H), 4.92-5.00 (m, 1H), 6.15 (s, 1H), 6.44 (d, 1H), 6.54 (d, 1H), 6.75 (t, 1H), 7.28 (d, 1H), 7.48 (d, 1H), 7.58-7.68 (m, 2H), 7.76 (d, 1H), 7.86 (dd, 1H), 8.58 (d, 1H).

### Example 12a

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylic acid Isomer 3

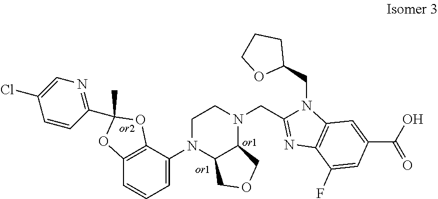 [SMILES: C[C@]1(c2ccc(Cl)cn2)Oc2cccc(N3CCN(Cc4nc5c(F)cc(C(=O)O)cc5n4C[C@@H]4CCCO4)[C@H]4COC[C@H]43)c2O1]

A sealed flask with methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylate, Isomer 3 Intermediate 126 (81 mg, 0.12 mmol) was evacuated and backfilled with N₂(g) (×3) and then 1,3,4,6,7,8-hexahydro-2H-pyrimido[1,2-a]pyrimidine (34 mg, 0.24 mmol) was added. A mixture of MeCN (3 mL) and water (0.6 mL) was degassed by N₂ (g) bubbling for 15 min, and then added. The reaction mixture was evacuated and backfilled with N₂(g) (×3) and then stirred at rt overnight. The reaction mixture was concentrated at reduced pressure, the residue was dissolved in DMSO, filtered and purified by preparative HPLC, PrepMethod A (gradient: 20-90%) to give the title compound Isomer 3 (64 mg, 80%); MS (ESI) m/z [M+H]⁺ 650.53; ¹H NMR (500 MHz, CD₃OD) 1.51 (dq, 1H), 1.65-1.78 (m, 1H), 1.78-1.9 (m, 1H), 1.96-2.09 (m, 4H), 2.54 (dt, 1H), 2.79 (d, 1H), 3.09-3.2 (m, 3H), 3.50 (t, 1H), 3.62-3.79 (m, 4H), 3.90 (dd, 1H), 4.20-4.34 (m, 3H), 4.44 (d, 1H), 4.64 (td, 1H), 4.97 (dd, 1H), 6.30 (d, 1H), 6.47 (d, 1H), 6.70 (t, 1H), 7.54 (d, 1H), 7.63 (d, 1H), 7.70 (dd, 1H), 7.97 (d, 1H), 8.53 (d, 1H).

### Example 12b

### 2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylic acid Isomer 4

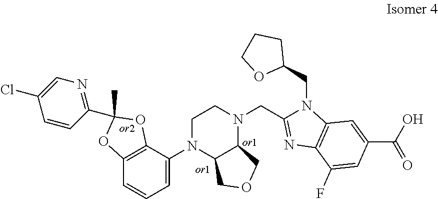 [SMILES: C[C@]1(c2ccc(Cl)cn2)Oc2cccc(N3CCN(Cc4nc5c(F)cc(C(=O)O)cc5n4C[C@@H]4CCCO4)[C@H]4COC[C@H]43)c2O1]

The title compound was prepared as described for Example 12a starting from methyl 2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylate, Isomer 4 Intermediate 127 (83 mg, 0.12 mmol) to give the title compound Isomer 4 (63 mg, 78%); MS (ESI) m/z [M+H]⁺ 648.5; ¹H NMR (500 MHz, CD₃OD) 1.59 (dq, 1H), 1.77 (td, 1H), 1.82-1.91 (m, 1H), 2.05-2.14 (m, 1H), 2.15 (s, 3H), 2.52 (dt, 1H), 2.76-2.83 (m, 1H), 3.12 (s, 1H), 3.18-3.24 (m, 2H), 3.63-3.84 (m, 4H), 3.84-3.94 (m, 2H), 4.26-4.34 (m, 2H), 4.39 (dd, 1H), 4.46 (dd, 1H), 4.64 (td, 1H), 5.01 (dd, 1H), 6.36 (dd, 1H), 6.44-6.5 (m, 1H), 6.69-6.76 (m, 1H), 7.58-7.66 (m, 2H), 7.79 (dt, 1H), 8.05-8.1 (m, 1H), 8.55 (d, 1H).

### Example 13a

### rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid Isomer 1

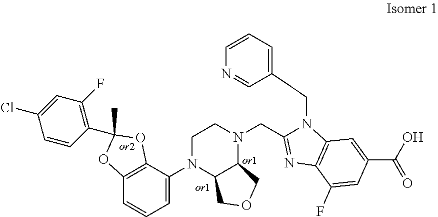 [SMILES: C[C@]1(c2ccc(Cl)cc2F)Oc2cccc(N3CCN(Cc4nc5c(F)cc(C(=O)O)cc5n4Cc4cccnc4)[C@H]4COC[C@H]43)c2O1]

LiOH monohydrate (3.2 mg, 72 μmol) was added to a solution of rel-methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylate, Isomer 1 Intermediate 128 (20 mg, 29 μmol) in THF:H₂O (2:1, 1 mL) and the reaction mixture was stirred at rt for 16 h. The reaction mixture was purified by preparative HPLC, PrepMethod D (gradient: 0-40%) to give the lithium salt of the title compound Isomer 1 (13.7 mg, 73%); HRMS (ESI) m/z [M+H]⁺ calcd for C₃₅H₃₁ClF₂N₅O₅: 674.1976, found: 674.1972; ¹H NMR (600 MHz, DMSO-d₆) δ 8.46-8.28 (m, 2H), 7.64 (s, 1H), 7.56-7.44 (m, 3H), 7.38 (d, 1H), 7.35-7.23 (m, 2H), 6.70 (t, 1H), 6.52 (d, 1H), 6.25 (d, 1H), 5.64 (d, 2H), 4.45-4.40 (m, 1H), 4.33 (d, 1H), 4.12 (d, 1H), 3.71 (dd, 1H), 3.52 (d, 1H), 3.15-3.10 (m, 2H), 3.02-2.89 (m, 2H), 2.74-2.62 (m, 2H), 2.37-2.30 (m, 1H), 2.00 (s, 3H).

### Example 13b

### rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid Isomer 2

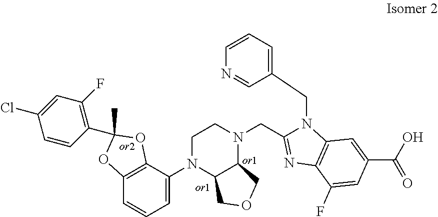 [SMILES: C[C@]1(c2ccc(Cl)cc2F)Oc2cccc(N3CCN(Cc4nc5c(F)cc(C(=O)O)cc5n4Cc4cccnc4)[C@H]4COC[C@H]43)c2O1]

The title compound was prepared as described for Example 13a from rel-methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylate, Isomer 2 Intermediate 129 (5.3 mg, 7.7 μmol) to give the lithium salt of the title compound Isomer 2 (4.6 mg, 82%); HRMS (ESI) m/z [M+H]⁺ calcd for C₃₅H₃₁ClF₂N₅O₅: 674.1976, found: 674.1988; ¹H NMR (600 MHz, DMSO-d₆) δ 8.41 (dd, 1H), 8.35 (d, 1H), 7.64 (s, 1H), 7.56-7.44 (m, 3H), 7.38 (d, 1H), 7.34-7.23 (m, 2H), 6.70 (t, 1H), 6.52 (d, 1H), 6.25 (d, 1H), 5.64 (d, 2H), 4.42 (td, 1H), 4.32 (d, 1H), 4.12 (d, 1H), 3.71 (dd, 1H), 3.52 (d, 1H), 3.15-3.10 (m, 2H), 3.02-2.90 (m, 2H), 2.76-2.62 (m, 2H), 2.33 (t, 1H), 2.01 (s, 3H).

### Example 13c

### rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid Isomer 4

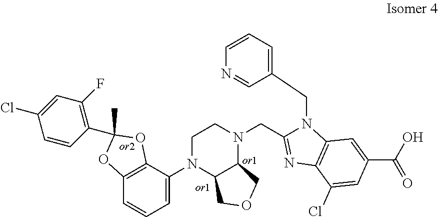 [SMILES: C[C@]1(c2ccc(Cl)cc2F)Oc2cccc(N3CCN(Cc4nc5c(Cl)cc(C(=O)O)cc5n4Cc4cccnc4)[C@H]4COC[C@H]43)c2O1]

The title compound was prepared as described for Example 13a from rel-methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylate, Isomer 4 Intermediate 130 (7.5 mg, 11 μmol) to give the lithium salt of the title compound Isomer 4 (3.9 mg, 56%); MS (ESI) m/z [M+H]⁺ 674.0; ¹H NMR (600 MHz, DMSO-d₆) δ 8.41 (dd, 1H), 8.35 (d, 1H), 7.64 (s, 1H), 7.56-7.42 (m, 3H), 7.43-7.34 (m, 1H), 7.35-7.24 (m, 2H), 6.70 (t, 1H), 6.51 (d, 1H), 6.26 (d, 1H), 5.64 (s, 2H), 4.41 (q, 1H), 4.32 (d, 1H), 4.15-4.05 (m, 1H), 3.71 (dd, 1H), 3.52 (d, 1H), 3.18-3.08 (m, 2H), 3.03-2.91 (m, 2H), 2.68 (q, 2H), 2.35-2.30 (m, 1H), 2.00 (s, 3H).

### Example 14a

### 4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid Isomer 1

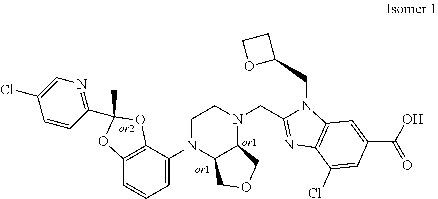 [SMILES: C[C@]1(c2ccc(Cl)cn2)Oc2cccc(N3CCN(Cc4nc5c(Cl)cc(C(=O)O)cc5n4C[C@@H]4CCO4)[C@H]4COC[C@H]43)c2O1]

A mixture of methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 1 Intermediate 131 (39 mg, 58 μmol) and LiOH monohydrate (7.6 mg, 0.17 mmol) in THF:H₂O (1:3, 4 mL) was stirred at 20° C. overnight. The reaction mixture was concentrated under reduced pressure and acidified with 1 M citric acid (aq). The product was collected by filtration, washed with water (3×75 mL), and then dried in vacuo at 60° C. to give the title compound Isomer 1 (19 mg, 51%); HRMS (ESI) m/z [M+H]⁺ calcd for C₃₂H₃₂Cl₂N₅O₆: 652.1724, found: 652.1796; ¹H NMR (600 MHz, DMSO-d₆) δ 13.08 (s, 1H), 8.67 (d, 1H), 8.25 (s, 1H), 7.99 (dd, 1H), 7.79 (s, 1H), 7.59 (d, 1H), 6.72 (t, 1H), 6.51 (d, 1H), 6.39 (d, 1H), 5.13 (q, 1H), 4.85 (dd, 1H), 4.70 (dd, 1H), 4.53 (q, 1H), 4.47-4.39 (m, 2H), 4.17 (dd, 1H), 4.06 (d, 1H), 3.73 (dd, 1H), 3.67-3.56 (m, 2H), 3.35 (t, 1H), 3.14-3.04 (m, 3H), 2.70 (d, 1H), 2.64-2.61 (m, 1H), 2.45-2.38 (m, 1H), 2.24 (p, 1H), 2.00 (s, 3H).

### Example 14b

### 4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid Isomer 2

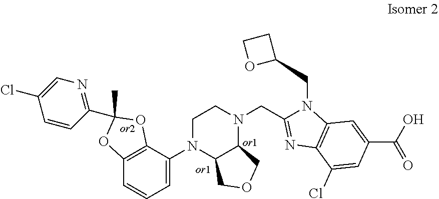 [SMILES: C[C@]1(c2ccc(Cl)cn2)Oc2cccc(N3CCN(Cc4nc5c(Cl)cc(C(=O)O)cc5n4C[C@@H]4CCO4)[C@H]4COC[C@H]43)c2O1]

The title compound was prepared as described in Example 14a from methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 2 Intermediate 132 (35 mg, 53 μmol) to give the title compound Isomer 2 (14 mg, 42%); HRMS (ESI) m/z [M+H]⁺ calcd for C₃₂H₃₂Cl₂N₅O₆: 652.1724, found: 652.1742; ¹H NMR (600 MHz, DMSO-d₆) δ 13.13 (s, 1H), 8.68 (d, 1H), 8.24 (s, 1H), 8.01 (dd, 1H), 7.78 (s, 1H), 7.61 (d, 1H), 6.73 (t, 1H), 6.51 (d, 1H), 6.40 (d, 1H), 5.05-4.95 (m, 2H), 4.66-4.40 (m, 5H), 4.24 (d, 1H), 3.80 (dd, 1H), 3.65 (t, 1H), 3.50 (d, 1H), 3.37 (t, 1H), 3.21-2.97 (m, 3H), 2.70-2.62 (m, 2H), 2.38 (d, partly overlapping with solvent), 2.01 (s, 3H).

### Example 14c

### 4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid Isomer 3

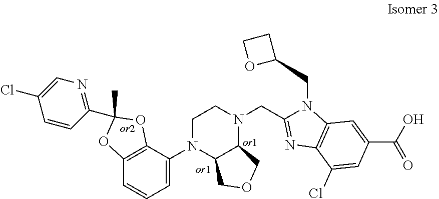 [SMILES: C[C@]1(c2ccc(Cl)cn2)Oc2cccc(N3CCN(Cc4nc5c(Cl)cc(C(=O)O)cc5n4C[C@@H]4CCO4)[C@H]4COC[C@H]43)c2O1]

The title compound was prepared as described in Example 14a from methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 3 Intermediate 133 (29 mg, 44 μmol) to give the title compound Isomer 3 (24 mg, 85%); HRMS (ESI) m/z [M+H]⁺ calcd for C₃₂H₃₂Cl₂N₅O₆: 652.1724, found: 652.1722; ¹H NMR (600 MHz, DMSO-d₆) δ 13.13 (s, 1H), 8.70 (d, 1H), 8.25 (s, 1H), 7.99 (dd, 1H), 7.79 (s, 1H), 7.61 (d, 1H), 6.72 (t, 1H), 6.50 (d, 1H), 6.41 (d, 1H), 5.15 (q, 1H), 4.96-4.81 (m, 1H), 4.70 (dd, 1H), 4.59-4.36 (m, 3H), 4.21-4.02 (m, 2H), 3.85-3.50 (m, 4H), 3.22 (d, 1H), 3.10-2.97 (m, 2H), 2.70 (d, 1H), 2.63 (s, 1H), 2.41 (d, 1H), 2.24 (p, 1H), 1.98 (s, 3H).

### Example 14d

### 4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid Isomer 4

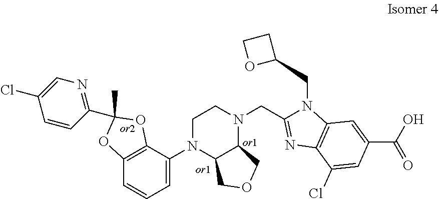 [SMILES: C[C@]1(c2ccc(Cl)cn2)Oc2cccc(N3CCN(Cc4nc5c(Cl)cc(C(=O)O)cc5n4C[C@@H]4CCO4)[C@H]4COC[C@H]43)c2O1]

The title compound Isomer 4 was prepared as described in Example 14a from methyl 4-chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 4 Intermediate 134 (23 mg, 35 μmol) to give the title compound Isomer 4 (21 mg, 94%); HRMS (ESI) m/z [M+H]⁺ calcd for C₃₂H₃₂Cl₂N₅O₆: 652.1724, found: 652.1770; ¹H NMR (600 MHz, DMSO-d₆) δ 13.12 (s, 1H), 8.70 (d, 1H), 8.26 (d, 1H), 7.99 (dd, 1H), 7.78 (d, 1H), 7.62 (t, 1H), 6.73 (t, 1H), 6.51 (t, 1H), 6.42 (d, 1H), 5.14-4.91 (m, 2H), 4.68-4.41 (m, 5H), 4.26 (d, 1H), 3.93-3.66 (m, 3H), 3.50 (d, 1H), 3.15-2.94 (m, 2H), 2.77-2.57 (m, 4H), 2.42-2.36 (m, 1H), 1.99 (s, 3H).

### Example 15a

### 4-Chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid Isomer 1

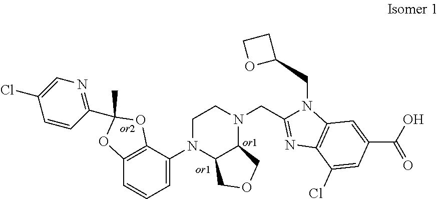 [SMILES: C[C@]1(c2ccc(Cl)cn2)Oc2cccc(N3CCN(Cc4nc5c(Cl)cc(C(=O)O)cc5n4C[C@@H]4CCO4)[C@H]4COC[C@H]43)c2O1]

LiOH hydrate (4.2 mg, 96 μmol, 1.5 eq) was added to a solution of methyl 4-chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 1 Intermediate 135 (44 mg, 64 μmol) in THF:H₂O (2:1, 5 mL) and the reaction mixture was stirred at rt for 16 h. The reaction mixture was concentrated under reduced pressure and acidified with 1 M citric acid (aq). The product was collected by filtration, washed with water (3×75 mL), and then dried in vacuo at 60° C. to give the title compound Isomer 1 (32 mg, 74%); HRMS (ESI) m/z [M+H]⁺ calcd for C₃₃H₃₂Cl₂FN₄O₆: 669.1678, found: 669.1708; ¹H NMR (600 MHz, DMSO-d₆) δ 13.09 (s, 1H), 8.25 (d, 1H), 7.79 (d, 1H), 7.57-7.48 (m, 2H), 7.31 (dd, 1H), 6.72 (t, 1H), 6.53 (d, 1H), 6.37 (d, 1H), 5.17-5.08 (m, 1H), 4.86 (dd, 1H), 4.70 (dd, 1H), 4.57 (td, 1H), 4.49-4.36 (m, 2H), 4.16 (dt, 1H), 4.11-4.03 (m, 1H), 3.74 (dd, 1H), 3.66-3.54 (m, 2H), 3.17-3.00 (m, 3H), 2.71 (dt, 1H), 2.64-2.58 (m, 1H), 2.43 (td, 1H), 2.27-2.20 (m, 1H), 2.02 (s, 3H).

### Example 15b

### 4-Chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid Isomer 2

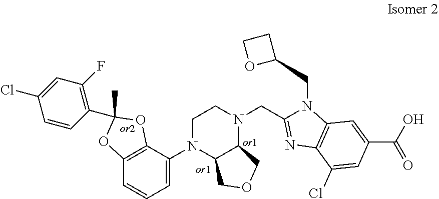 [SMILES: C[C@]1(c2ccc(Cl)cc2F)Oc2cccc(N3CCN(Cc4nc5c(Cl)cc(C(=O)O)cc5n4C[C@@H]4CCO4)[C@H]4COC[C@H]43)c2O1]

The title compound was prepared as described in Example 15a from methyl 4-chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 2 Intermediate 136 (14 mg, 20 μmol) to give the title compound Isomer 2 (12 mg, 90%) HRMS (ESI) m/z [M+H]⁺ calcd for C₃₃H₃₂Cl₂FN₄O₆: 669.1678, found: 669.1712; ¹H NMR (600 MHz, DMSO-d₆) δ 13.11 (s, 1H), 8.26 (s, 1H), 7.80 (s, 1H), 7.60-7.47 (m, 2H), 7.32 (dd, 1H), 6.71 (t, 1H), 6.50 (d, 1H), 6.40 (d, 1H), 5.16 (q, 1H), 4.89 (d, 1H), 4.71 (dd, 1H), 4.57-4.38 (m, 3H), 4.15 (dt, 1H), 4.10 (d, 1H), 3.80-3.66 (m, 3H), 3.61 (d, 1H), 3.11-2.99 (m, 2H), 2.70 (d, 1H), 2.63 (s, 1H), 2.44 (t, 1H), 2.30-2.18 (m, 1H), 1.99 (s, 3H).

### Example 15c

### 4-Chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid Isomer 3

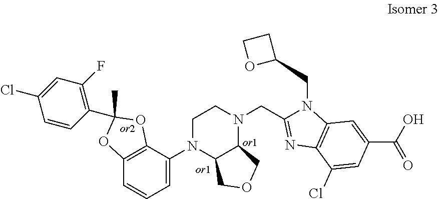 [SMILES: C[C@]1(c2ccc(Cl)cc2F)Oc2cccc(N3CCN(Cc4nc5c(Cl)cc(C(=O)O)cc5n4C[C@@H]4CCO4)[C@H]4COC[C@H]43)c2O1]

The title compound was prepared as described in Example 15a from methyl 4-chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 3 Intermediate 137 (46 mg, 67 μmol) to give the title compound Isomer 3 (28 mg, 62%); HRMS (ESI) m/z [M+H]⁺ calcd for C₃₃H₃₂Cl₂FN₄O₆: 669.1678, found: 669.1712; ¹H NMR (600 MHz, DMSO-d₆) δ 13.08 (s, 1H), 8.25 (d, 1H), 7.78 (d, 1H), 7.58-7.51 (m, 2H), 7.32 (dd, 1H), 6.73 (t, 1H), 6.54 (d, 1H), 6.39 (d, 1H), 5.00 (ddd, 2H), 4.67 (td, 1H), 4.59-4.41 (m, 4H), 4.27 (d, 1H), 3.81 (dd, 1H), 3.64 (t, 1H), 3.51 (d, 1H), 3.22-3.10 (m, 1H), 3.11-2.99 (m, 2H), 2.74-2.62 (m, 2H), 2.46-2.34 (m, 2H), 2.02 (s, 3H).

### Example 15d

### 4-Chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid Isomer 4

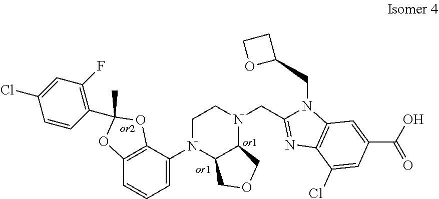 [SMILES: C[C@]1(c2ccc(Cl)cc2F)Oc2cccc(N3CCN(Cc4nc5c(Cl)cc(C(=O)O)cc5n4C[C@@H]4CCO4)[C@H]4COC[C@H]43)c2O1]

The title compound Isomer 4 was prepared as described in Example 15a from methyl 4-chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylate, Isomer 4 Intermediate 138 (13 mg, 19 μmol) to give the title compound Isomer 4 (10 mg, 86%); HRMS (ESI) m/z [M+H]⁺ calcd for C₃₃H₃₂Cl₂FN₄O₆: 669.1678, found: 669.1698; ¹H NMR (600 MHz, DMSO-d₆) δ 13.08 (s, 1H), 8.23 (s, 1H), 7.78 (s, 1H), 7.62-7.46 (m, 2H), 7.32 (dd, 1H), 6.72 (t, 1H), 6.50 (d, 1H), 6.42 (d, 1H), 5.09-4.93 (m, 2H), 4.66-4.42 (m, 5H), 4.28 (d, 1H), 3.89-3.67 (m, 3H), 3.50 (d, 1H), 3.34 (d, 2H), 3.08 (t, 1H), 3.00 (t, 1H), 2.67 (dd, 2H), 2.45-2.38 (m, 1H), 1.99 (s, 3H).

### Example 16a

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid Isomer 1

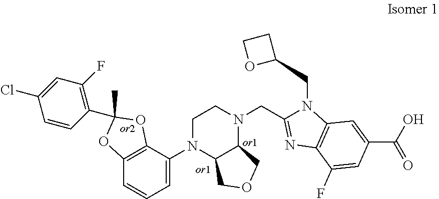 [SMILES: C[C@]1(c2ccc(Cl)cc2F)Oc2cccc(N3CCN(Cc4nc5c(F)cc(C(=O)O)cc5n4C[C@@H]4CCO4)[C@H]4COC[C@H]43)c2O1]

LiOH hydrate (5.1 mg, 0.12 mmol) was added in one portion to a solution of methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 1 Intermediate 139 (31 mg, 46 μmol) in a mixture of THF:H₂O (1:1, 5 mL) and the reaction mixture was stirred at rt for 24 h. The reaction mixture was concentrated, and the residue were acidified using 1 M citric acid (aq, 4 mL). The reaction mixture was stirred at ambient temperature overnight. The obtained solid was filtered off and dried in vacuo for 2 h to give the title compound Isomer 1 (29 mg, 96%); HRMS (ESI) m/z [M+H]⁺ calcd for C₃₃H₃₂ClF₂N₄O₆: 653.1972, found: 653.1988; ¹H NMR (600 MHz, DMSO-d₆) δ 13.05 (s, 1H), 8.14 (s, 1H), 7.58-7.45 (m, 3H), 7.31 (dd, 1H), 6.72 (t, 1H), 6.53 (d, 1H), 6.37 (d, 1H), 5.13 (dt, 1H), 4.84 (d, 1H), 4.69 (dd, 1H), 4.57 (t, 1H), 4.48-4.37 (m, 2H), 4.19-4.12 (m, 1H), 4.08 (d, 1H), 3.74 (dd, 1H), 3.65-3.53 (m, 2H), 3.15-3.01 (m, 3H), 2.77-2.68 (m, 1H), 2.61 (d, 1H), 2.44-2.39 (m, 2H), 2.25 (p, 1H), 2.02 (s, 3H).

### Example 16b

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid Isomer 2

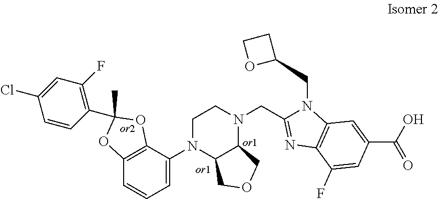 [SMILES: C[C@]1(c2ccc(Cl)cc2F)Oc2cccc(N3CCN(Cc4nc5c(F)cc(C(=O)O)cc5n4C[C@@H]4CCO4)[C@H]4COC[C@H]43)c2O1]

The title compound was prepared as described for Example 16a from methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 2 Intermediate 140 (12 mg, 18 μmol) to give the title compound Isomer 2 (11 mg, 97%); HRMS (ESI) m/z [M+H]⁺ calcd for C₃₃H₃₂ClF₂N₄O₆: 653.1972, found: 653.1954; ¹H NMR (600 MHz, DMSO-d₆) δ 13.05 (s, 1H) 8.15 (s, 1H), 7.61-7.46 (m, 3H), 7.36-7.27 (m, 1H), 6.71 (t, 1H), 6.50 (d, 1H), 6.40 (d, 1H), 5.16 (q, 1H), 4.93-4.83 (m, 1H), 4.70 (dd, 1H), 4.57-4.40 (m, 3H), 4.16 (q, 1H), 4.09 (d, 1H), 3.81-3.66 (m, 3H), 3.59 (d, 1H), 3.12-3.00 (m, 2H), 2.71 (d, 1H), 2.61 (d, 2H), 2.43 (d, 1H), 2.25 (p, 1H), 1.99 (s, 3H).

### Example 16c

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid Isomer 3

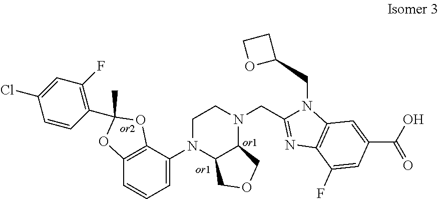 [SMILES: C[C@]1(c2ccc(Cl)cc2F)Oc2cccc(N3CCN(Cc4nc5c(F)cc(C(=O)O)cc5n4C[C@@H]4CCO4)[C@H]4COC[C@H]43)c2O1]

The title compound was prepared as described for Example 16a from methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 3 Intermediate 141 (28 mg, 42 μmol) to give the title compound Isomer 3 (24 mg, 88%); HRMS (ESI) m/z [M+H]⁺ calcd for C₃₃H₃₂ClF₂N₄O₆: 653.1972, found: 653.1996; ¹H NMR (600 MHz, DMSO-d₆) δ 13.04 (s, 1H), 8.13 (s, 1H), 7.60-7.46 (m, 3H), 7.32 (dd, 1H), 6.73 (t, 1H), 6.54 (d, 1H), 6.39 (d, 1H), 5.08-4.91 (m, 2H), 4.66 (td, 1H), 4.60-4.41 (m, 4H), 4.26 (d, 1H), 3.81 (dd, 1H), 3.64 (t, 1H), 3.48 (d, 1H), 3.16 (d, 1H), 3.12-2.99 (m, 2H), 2.70-2.65 (m, 2H), 2.45-2.35 (m, 3H), 2.02 (s, 3H).

### Example 16d

### 2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid Isomer 4

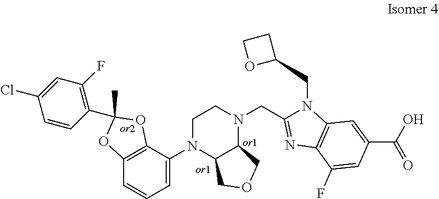 [SMILES: C[C@]1(c2ccc(Cl)cc2F)Oc2cccc(N3CCN(Cc4nc5c(F)cc(C(=O)O)cc5n4C[C@@H]4CCO4)[C@H]4COC[C@H]43)c2O1]

The title compound was prepared as described for Example 16a from methyl 2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylate, Isomer 4 Intermediate 142 (11 mg, 16 μmol) to give the title compound Isomer 4 (10 mg, 86%); HRMS (ESI) m/z [M+H]⁺ calcd for C₃₃H₃₂ClF₂N₄O₆: 653.1972, found: 653.1996; ¹H NMR (600 MHz, DMSO-d₆) δ 13.04 (s, 1H), 8.14 (s, 1H), 7.64-7.45 (m, 3H), 7.32 (dd, 1H), 6.72 (t, 1H), 6.50 (d, 1H), 6.42 (d, 1H), 5.11-4.90 (m, 2H), 4.63-4.41 (m, 6H), 4.28 (d, 1H), 3.87-3.69 (m, 3H), 3.48 (d, 1H), 3.14-2.95 (m, 2H), 2.74-2.62 (m, 2H), 2.45-2.38 (m, 2H), 1.99 (s, 3H).

### Example 17

### rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylic acid Isomer 1

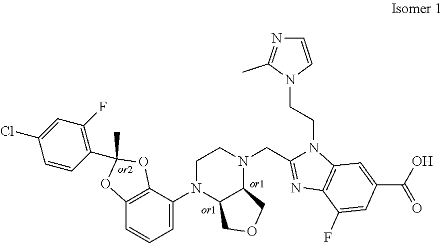 [SMILES: Cc1nccn1CCn1c(CN2CCN(c3cccc4c3O[C@@](C)(c3ccc(Cl)cc3F)O4)[C@@H]3COC[C@@H]32)nc2c(F)cc(C(=O)O)cc21]

LiOH monohydrate (3.2 mg, 74 μmol) was added to a solution of rel-methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylate, Isomer 1 Intermediate 143 (26 mg, 37 μmol) in THF:H₂O (2:1, 5 mL) and the reaction mixture was stirred at rt for 16 h. The reaction mixture was acidified to pH 3 with 1 M NaH₂PO₄ (aq) and diluted with water. The precipitate was collected by filtration, washed with water and dried in vacuo to give the title compound Isomer 1 (10 mg, 47%); HRMS (ESI) m/z [M+H]⁺ calcd for C₃₅H₃₄ClF₂N₆O₅: 691.2242, found: 691.2302; ¹H NMR (500 MHz, DMSO-d₆) δ 13.01 (s, 1H), 7.63 (s, 1H), 7.58-7.43 (m, 3H), 7.31 (dd, 1H), 6.96 (s, 1H), 6.72 (dd, 2H), 6.54 (d, 1H), 6.38 (d, 1H), 4.74 (d, 2H), 4.57 (q, 1H), 4.38 (t, 2H), 4.13 (d, 1H), 3.99 (d, 1H), 3.76 (dd, 1H), 3.59 (t, 1H), 3.41 (dd, 2H), 3.17-3.02 (m, 2H), 2.78-2.68 (m, 1H), 2.66-2.56 (m, 1H), 2.40 (d, 1H), 2.02 (s, 3H), 1.80 (s, 3H).

### Example 18

### rel-4-Chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylic acid Isomer 1

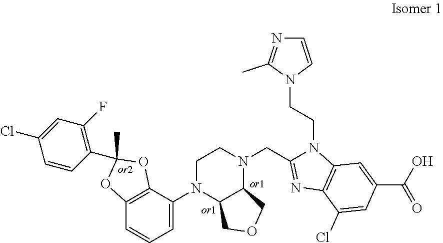 [SMILES: Cc1nccn1CCn1c(CN2CCN(c3cccc4c3O[C@@](C)(c3ccc(Cl)cc3F)O4)[C@@H]3COC[C@@H]32)nc2c(Cl)cc(C(=O)O)cc21]

LiOH hydrate (2.4 mg, 54 μmol) was added in one portion to a solution of rel-methyl 4-chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylate Isomer 1 Intermediate 144 (16 mg, 22 μmol) in THF:H₂O (1:1, 5 mL) and the reaction mixture was stirred at rt for 24 h. The reaction mixture was concentrated in vacuo and the residue was dissolved in DMSO (5 mL) and purified by preparative HPLC, PrepMethod D (gradient: 60-85%) to give the lithium salt of the title compound Isomer 1 (8 mg, 50%); HRMS (ESI) m/z [M+H]⁺ calcd for C₃₅H₃₄Cl₂FN₆O₅: 707.1946, found: 707.1946 ¹H NMR (600 MHz, DMSO-d₆) δ 7.88 (s, 1H), 7.79 (d, 1H), 7.58-7.50 (m, 2H), 7.31 (dd, 1H), 6.84 (d, 1H), 6.74-6.66 (m, 2H), 6.53 (d, 1H), 6.37 (d, 1H), 4.67 (t, 2H), 4.52 (td, 1H), 4.38-4.25 (m, 2H), 4.02 (d, 1H), 3.72 (dd, 1H), 3.66 (d, 1H), 3.57 (t, 1H), 3.09 (td, 2H), 3.01 (d, 2H), 2.64 (d, 1H), 2.33 (t, 2H), 2.01 (s, 3H), 1.85 (s, 3H).

### Example 19

### rel-4-Chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylic acid Isomer 4

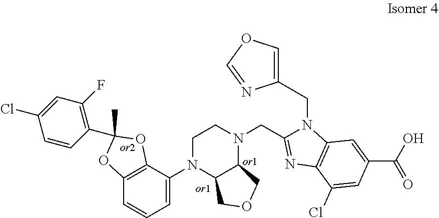 [SMILES: C[C@]1(c2ccc(Cl)cc2F)Oc2cccc(N3CCN(Cc4nc5c(Cl)cc(C(=O)O)cc5n4Cc4cocn4)[C@H]4COC[C@H]43)c2O1]

LiOH hydrate (7.9 mg, 0.18 mmol) was added in one portion to a solution of rel-methyl 4-chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylate, Isomer 4 Intermediate 145 (50 mg, 72 μmol) in THF:H₂O (1:1, 5 mL) and the reaction mixture was stirred at rt for 24 h. The reaction mixture was concentrated, and the residue was acidified by addition of 1 M citric acid (aq, 4 mL) and stirred at ambient temperature for 8 h. The obtained solid was filtered off and dried in vacuo for 2 h to give the title compound Isomer 4 (44 mg, 91%); MS (ESI) m/z [M+H]⁺ 682.0; ¹H NMR (500 MHz, DMSO-d₆) δ 13.14 (s, 1H), 8.32 (s, 1H), 8.22 (s, 1H), 8.14 (s, 1H), 7.78 (s, 1H), 7.53 (dd, 2H), 7.30 (d, 1H), 6.72 (t, 1H), 6.53 (d, 1H), 6.35 (d, 1H), 5.79-5.57 (m, 2H), 4.56 (d, 2H), 4.22 (d, 1H), 3.78 (dd, 1H), 3.67 (d, 1H), 3.53 (t, 1H), 3.42 (dd, 2H), 3.10 (d, 2H), 2.99 (t, 1H), 2.71 (d, 1H), 2.02 (s, 3H).

### Example 20a

### rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylic acid Isomer 1

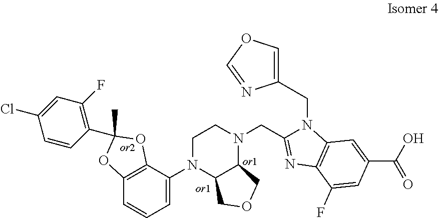 [SMILES: C[C@]1(c2ccc(Cl)cc2F)Oc2cccc(N3CCN(Cc4nc5c(F)cc(C(=O)O)cc5n4Cc4cocn4)[C@H]4COC[C@H]43)c2O1]

LiOH hydrate (3.7 mg, 0.084 mmol) was added in one portion to a solution of rel-methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylate, Isomer 1 Intermediate 146 (23 mg, 34 μmol) in THF:H₂O (1:1, 5 mL) and the reaction mixture was stirred at rt for 24 h. The reaction mixture was concentrated in vacuo and the crude product was dissolved in DMSO (5 mL) and purified by preparative HPLC, PrepMethod D (gradient: 30-50%) to give the lithium salt of the title compound (7.3 mg, 36%); HRMS (ESI) m/z [M+H]⁺ calcd for C₃₃H₂₉ClF₂N₅O₆: 664.1768, found: 664.1780; ¹H NMR (600 MHz, DMSO-d₆) δ 8.32 (d, 1H), 8.10 (s, 1H), 7.85 (d, 1H), 7.59-7.50 (m, 2H), 7.44 (d, 1H), 7.32 (dd, 1H), 6.71 (t, 1H), 6.53-6.45 (m, 1H), 6.39 (d, 1H), 5.65 (d, 1H), 5.51 (d, 1H), 4.59-4.44 (m, 2H), 4.24 (d, 1H), 3.79 (dd, 1H), 3.66 (d, 2H), 3.56 (d, 1H), 3.29-3.22 (m, 1H), 3.07-2.95 (m, 2H), 2.74 (d, 1H), 2.41 (t, 1H), 1.98 (s, 3H).

### Example 20b

### rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylic acid Isomer 4

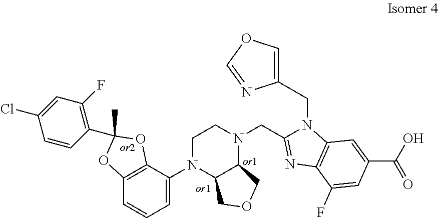 [SMILES: C[C@]1(c2ccc(Cl)cc2F)Oc2cccc(N3CCN(Cc4nc5c(F)cc(C(=O)O)cc5n4Cc4cocn4)[C@H]4COC[C@H]43)c2O1]

The title compound was prepared as described for Example 20a from rel-methyl 2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylate, Isomer 4 Intermediate 147 (10 mg, 15 μmol) to give the title compound Isomer 4 (4.9 mg, 47%); HRMS (ESI) m/z [M+H]⁺ calcd for C₃₃H₂₉ClF₂N₅O₆: 664.1768, found: 664.1766; ¹H NMR (600 MHz, DMSO-d₆) δ 8.31 (s, 1H), 8.07 (s, 1H), 7.84 (s, 1H), 7.58-7.48 (m, 2H), 7.43 (d, 1H), 7.30 (dd, 1H), 6.72 (t, 1H), 6.53 (d, 1H), 6.35 (d, 1H), 5.62 (d, 1H), 5.49 (d, 1H), 4.59-4.43 (m, 3H), 4.20 (d, 1H), 3.78 (dd, 1H), 3.63-3.47 (m, 2H), 3.28-3.22 (m, 1H), 3.15-2.95 (m, 2H), 2.74 (d, 1H), 2.40 (d, 1H), 2.02 (s, 3H).

### Example 21

### rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylic acid Isomer 1

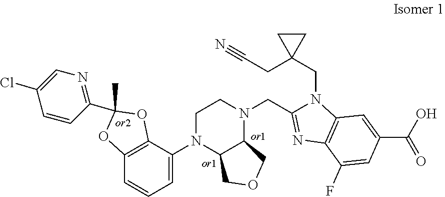 [SMILES: C[C@]1(c2ccc(Cl)cn2)Oc2cccc(N3CCN(Cc4nc5c(F)cc(C(=O)O)cc5n4CC4(CC#N)CC4)[C@H]4COC[C@H]43)c2O1]

LiOH hydrate (11.4 mg, 260 μmol) was added to a solution of rel-methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylate Isomer 1 Intermediate 148 (79 mg, 104 μmol) in THF:H₂O (2:1, 7 mL) and the reaction mixture was stirred at rt for 16 h. The reaction mixture was acidified to pH 3 using 10% NaH₂PO₄ (aq). The formed precipitate was filtered off and dried in vacuo to give the title compound Isomer 1 (70 mg, 92%) as a white solid; HRMS (ESI) m/z [M+H]⁺ calcd for C₃₄H₃₃ClFN₆O₅: 659.2180, found: 659.2134; ¹H NMR (600 MHz, DMSO-d₆) δ 8.67 (d, 1H), 8.05 (s, 1H), 7.99 (dd, 1H), 7.59 (d, 1H), 7.49 (d, 1H), 6.72 (t, 1H), 6.52 (d, 1H), 6.40 (d, 1H), 4.59 (d, 1H), 4.52 (d, 1H), 4.46 (q, 1H), 4.25 (d, 1H), 4.12 (dd, 1H), 3.79 (dd, 1H), 3.67 (d, 1H), 3.57 (t, 1H), 3.40 (t, 2H), 3.15-3.06 (m, 2H), 2.84 (dd, 1H), 2.76 (d, 1H), 2.60 (d, 2H), 2.00 (s, 3H), 0.74-0.63 (m, 4H).

### Example 22a

### rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid Isomer 1

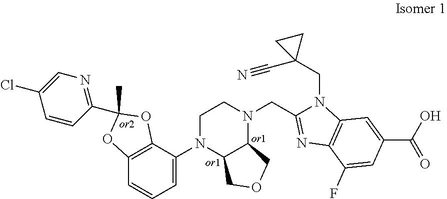 [SMILES: C[C@]1(c2ccc(Cl)cn2)Oc2cccc(N3CCN(Cc4nc5c(F)cc(C(=O)O)cc5n4CC4(C#N)CC4)[C@H]4COC[C@H]43)c2O1]

LiOH hydrate (10.2 mg, 0.23 mmol) was added to a solution of rel-methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylate, Isomer 1 Intermediate 149 (61 mg, 93 μmol) in THF:H₂O (2:1, 5 mL) and the reaction mixture was stirred at rt for 16 h. The reaction mixture was acidified to pH 3 with 10% NaH₂PO₄ (aq). The formed precipitate was filtered off and dried in vacuo to give the title compound Isomer 1 (40 mg, 67%) as a white solid; HRMS (ESI) m/z [M+H]⁺ calcd for C₃₃H₃₁ClFN₆O₅: 645.2022, found: 645.2012; ¹H NMR (600 MHz, DMSO-d₆) δ 13.00 (s, 1H), 8.68 (d, 1H), 8.09 (s, 1H), 8.00 (dd, 1H), 7.60 (d, 1H), 7.52 (d, 1H), 6.72 (t, 1H), 6.52 (d, 1H), 6.39 (d, 1H), 4.84 (d, 1H), 4.58 (d, 1H), 4.52 (q, 1H), 4.36 (d, 1H), 4.14 (dd, 1H), 3.80 (dd, 1H), 3.68 (d, 1H), 3.62 (t, 1H), 3.48-3.36 (m, 2H), 3.19-3.04 (m, 2H), 2.73 (d, 1H), 2.42 (d, 1H), 2.00 (s, 3H), 1.46-1.27 (m, 4H).

### Example 22b

### rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid Isomer 2

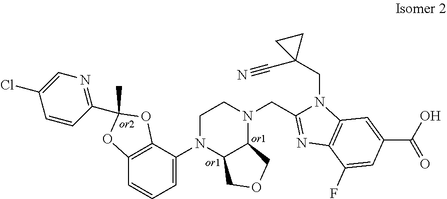 [SMILES: C[C@]1(c2ccc(Cl)cn2)Oc2cccc(N3CCN(Cc4nc5c(F)cc(C(=O)O)cc5n4CC4(C#N)CC4)[C@H]4COC[C@H]43)c2O1]

The title compound was prepared as described for Example 22a from rel-methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylate, Isomer 2 Intermediate 150 (43 mg, 0.65 mmol) to give the title compound Isomer 2 (27 mg, 64%) as a white solid; HRMS (ESI) m/z [M+H]⁺ calcd for C₃₃H₃₁ClFN₆O₅: 645.2022, found: 645.2040; ¹H NMR (600 MHz, DMSO-d₆) δ 13.01 (s, 1H), 8.70 (d, 1H), 8.14 (s, 1H), 7.99 (dd, 1H), 7.61 (d, 1H), 7.53 (d, 1H), 6.73 (t, 1H), 6.51 (d, 1H), 6.41 (d, 1H), 4.87 (d, 1H), 4.61 (d, 1H), 4.52 (q, 1H), 4.40 (d, 1H), 4.16 (d, 1H), 3.82 (dd, 1H), 3.69 (d, 1H), 3.63 (t, 1H), 3.43-3.38 (m, partly overlapping with solvent), 3.19-3.07 (m, partly overlapping with solvent), 2.73 (d, 1H), 2.43 (d, 1H), 1.99 (s, 3H), 1.48-1.27 (m, 4H).

### Example 22c

### rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid Isomer 3

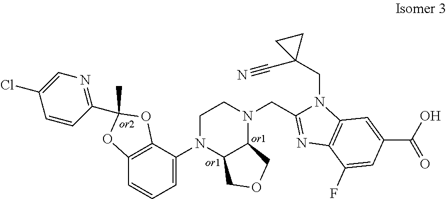 [SMILES: C[C@]1(c2ccc(Cl)cn2)Oc2cccc(N3CCN(Cc4nc5c(F)cc(C(=O)O)cc5n4CC4(C#N)CC4)[C@H]4COC[C@H]43)c2O1]

The title compound was prepared as described for Example 22a from rel-methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylate, Isomer 3 Intermediate 151 (67 mg, 101 μmol) to give the title compound Isomer 3 (41 mg, 67%) as a white solid; HRMS (ESI) m/z [M+H]⁺ calcd for C₃₃H₃₁ClFN₆O₅: 645.2022, found: 645.2014; ¹H NMR (600 MHz, DMSO-d₆) δ 13.00 (s, 1H), 8.68 (d, 1H), 8.08 (s, 1H), 8.00 (dd, 1H), 7.60 (d, 1H), 7.51 (d, 1H), 6.72 (t, 1H), 6.51 (d, 1H), 6.39 (d, 1H), 4.84 (d, 1H), 4.61 (d, 1H), 4.52 (q, 1H), 4.36 (d, 1H), 4.17 (d, 1H), 3.80 (dd, 1H), 3.71-3.60 (m, 2H), 3.19-3.08 (m, 3H), 2.73 (d, 1H), 2.46-2.40 (m, 1H), 2.00 (s, 3H), 1.42-1.28 (m, 4H).

### Example 23a

### rel-4-Chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid Isomer 1

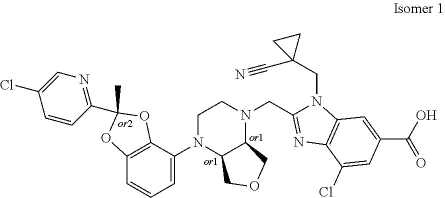 [SMILES: C[C@]1(c2ccc(Cl)cn2)Oc2cccc(N3CCN(Cc4nc5c(Cl)cc(C(=O)O)cc5n4CC4(C#N)CC4)[C@H]4COC[C@H]43)c2O1]

LiOH hydrate (3.3 mg, 74 μmol) was added to a solution rel-methyl 4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate Isomer 1 Intermediate 152 (20 mg, 30 μmol) in THF:H₂O (1:1, 4 mL) and the reaction mixture was stirred at rt for 24 h. The reaction mixture was acidified to pH 3 with 10% NaH₂PO₄ (aq). The formed precipitate was filtered off and dried in vacuo to give the title compound Isomer 1 (20 mg, 98%) as a white powder; HRMS (ESI) m/z [M+H]⁺ calcd for C₃₃H₃₁Cl₂N₆O₅: 661.1728, found: 661.1746; ¹H NMR (600 MHz, DMSO-d₆) δ 13.12 (s, 1H), 8.68 (d, 1H), 8.34 (d, 1H), 8.00 (dd, 1H), 7.84 (d, 1H), 7.60 (d, 1H), 6.72 (t, 1H), 6.52 (d, 1H), 6.39 (d, 1H), 4.88 (d, 1H), 4.66 (d, 1H), 4.53 (q, 1H), 4.39 (d, 1H), 4.14 (d, 1H), 3.80 (dd, 1H), 3.74 (d, 1H), 3.62 (t, 1H), 3.40 (t, 1H), 3.19-3.06 (m, 4H), 2.72 (d, 1H), 2.00 (s, 3H), 1.43-1.28 (m, 4H).

### Example 23b

### rel-4-Chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid Isomer 2

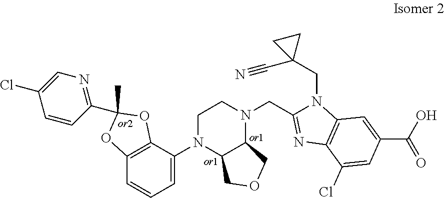 [SMILES: C[C@]1(c2ccc(Cl)cn2)Oc2cccc(N3CCN(Cc4nc5c(Cl)cc(C(=O)O)cc5n4CC4(C#N)CC4)[C@H]4COC[C@H]43)c2O1]

The title compound was prepared as described for Example 23a from rel-methyl 4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate Isomer 2 Intermediate 153 (14 mg, 21 μmol) to give the title compound Isomer 2 (13 mg, 95%); HRMS (ESI) m/z [M+H]⁺ calcd for C₃₃H₃₁Cl₂N₆O₅: 661.1728, found: 661.1752; ¹H NMR (600 MHz, DMSO-d₆) δ 13.12 (s, 1H), 8.70 (d, 1H), 8.35 (s, 1H), 7.99 (dd, 1H), 7.84 (s, 1H), 7.61 (d, 1H), 6.73 (t, 1H), 6.51 (d, 1H), 6.41 (d, 1H), 4.90 (d, 1H), 4.67 (d, 1H), 4.53 (q, 1H), 4.42 (d, 1H), 4.16 (d, 1H), 3.82 (dd, 1H), 3.79-3.63 (m, 3H), 3.26-3.03 (m, 4H), 2.70 (t, 1H), 1.99 (s, 3H), 1.47-1.28 (m, 4H).

### Example 24

### rel-4-Chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid Isomer 1

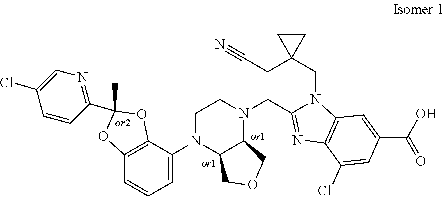 [SMILES: C[C@]1(c2ccc(Cl)cn2)Oc2cccc(N3CCN(Cc4nc5c(Cl)cc(C(=O)O)cc5n4CC4(CC#N)CC4)[C@H]4COC[C@H]43)c2O1]

LiOH hydrate (3.0 mg, 67 μmol) was added to a solution rel-methyl 4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylate Isomer 1 Intermediate 154 (19 mg, 27 μmol) in THF:H₂O (2:1, 4 mL) and the reaction mixture was stirred at rt 16 h. The reaction mixture was acidified to pH 3 with 10% NaH₂PO₄ (aq). The formed precipitate was filtered off and dried in vacuo to give the title compound Isomer 1 (16 mg, 92%) as a white solid; HRMS (ESI) m/z [M+H]⁺ calcd for C₃₄H₃₃Cl₂N₆O₅: 675.1884, found: 675.1918; ¹H NMR (600 MHz, DMSO-d₆) δ 13.14 (s, 1H), 8.67 (d, 1H), 8.23 (d, 1H), 7.99 (dd, 1H), 7.80 (d, 1H), 7.60 (d, 1H), 6.72 (t, 1H), 6.52 (d, 1H), 6.40 (d, 1H), 4.64-4.53 (m, 2H), 4.48 (q, 1H), 4.29 (d, 1H), 4.13 (dd, 1H), 3.79 (dd, 1H), 3.71 (d, 1H), 3.57 (t, 1H), 3.40 (t, 1H), 3.19-3.07 (m, 3H), 2.82 (d, 1H), 2.76 (d, 1H), 2.62-2.58 (m, 2H), 2.01 (s, 3H), 0.76-0.63 (m, 4H).

### Example 25

### rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid Isomer 1

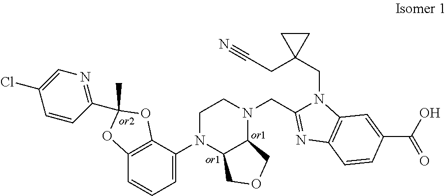 [SMILES: C[C@]1(c2ccc(Cl)cn2)Oc2cccc(N3CCN(Cc4nc5ccc(C(=O)O)cc5n4CC4(CC#N)CC4)[C@H]4COC[C@H]43)c2O1]

LiOH hydrate (27.7 mg, 0.63 mmol) was added in one portion to a solution of rel-methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylate Isomer 1 Intermediate 155 (137 mg, 0.21 mmol) in THF:H₂O (1:1, 2 mL) and the reaction mixture was stirred at rt for 24 h. The reaction mixture was concentrated in vacuo, diluted with water (3 mL) and acidified to pH 3 with 10% NaH₂PO₄ (aq). The formed precipitate was filtered off and dried in vacuo to give the title compound Isomer 1 (101 mg, 87%); HRMS (ESI) m/z [M+H]⁺ calcd for C₃₄H₃₄ClN₆O₅: 641.2274, found: 641.2308; ¹H NMR (500 MHz, DMSO-d₆) δ 12.80 (s, 1H), 8.67 (dd, 1H), 8.23 (d, 1H), 8.10-7.94 (m, 1H), 7.81 (dd, 1H), 7.66 (d, 1H), 7.59 (dd, 1H), 6.72 (t, 1H), 6.52 (dd, 1H), 6.39 (d, 1H), 4.64-4.50 (m, 2H), 4.46 (q, 1H), 4.25 (d, 1H), 4.15-4.06 (m, 1H), 3.79 (dd, 1H), 3.67 (d, 1H), 3.57 (t, 1H), 3.39 (t, 1H), 3.19-3.06 (m, 3H), 2.84 (d, 1H), 2.73 (d, 1H), 2.65-2.56 (m, 1H), 2.44 (s, 1H), 2.00 (s, 3H), 0.73-0.62 (m, 4H).

### Example 26

### rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid Isomer 1

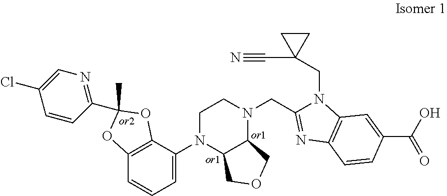 [SMILES: C[C@]1(c2ccc(Cl)cn2)Oc2cccc(N3CCN(Cc4nc5ccc(C(=O)O)cc5n4CC4(C#N)CC4)[C@H]4COC[C@H]43)c2O1]

LiOH hydrate (17.3 mg, 394 μmol) was added in one portion to a solution of rel-methyl 2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylate Isomer 1 Intermediate 156 (101 mg, 158 μmol) in THF:H₂O (1:1, 2 mL) and the reaction mixture was stirred at rt for 24 h. The reaction mixture was acidified to pH 3 with 10%. NaH₂PO₄ (aq). The formed precipitate was filtered off and dried in vacuo to give the title compound Isomer 1 (82 mg, 87%) as a beige solid; HRMS (ESI) m/z [M+H]⁺ calcd for C₃₃H₃₂ClN₆O₅: 627.2118, found: 627.2176; ¹H NMR (400 MHz, DMSO-d₆) δ 12.85 (s, 1H), 8.69 (d, 1H), 8.35 (s, 1H), 8.01 (dd, 1H), 7.85 (dd, 1H), 7.70 (d, 1H), 7.60 (d, 1H), 6.73 (t, 1H), 6.53 (d, 1H), 6.40 (d, 1H), 4.86 (d, 1H), 4.67-4.48 (m, 2H), 4.38 (d, 1H), 4.14 (d, 1H), 3.89-3.75 (m, 1H), 3.75-3.58 (m, 2H), 3.40 (t, 1H), 3.20-3.09 (m, 3H), 2.75 (d, 1H), 2.44 (s, 1H), 2.01 (s, 3H), 1.44-1.29 (m, 4H).

### Example 27

### rel-1-(2-(1H-Pyrazol-1-yl)ethyl)-4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid

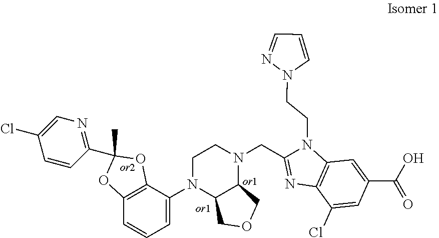 [SMILES: C[C@]1(c2ccc(Cl)cn2)Oc2cccc(N3CCN(Cc4nc5c(Cl)cc(C(=O)O)cc5n4CCn4cccn4)[C@H]4COC[C@H]43)c2O1]

A solution of rel-methyl 1-(2-(1H-pyrazol-1-yl)ethyl)-4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylate Isomer 1 Intermediate 157 (130 mg, 0.19 mmol) and 1,3,4,6,7,8-hexahydro-2H-pyrimido[1,2-a]pyrimidine (53 mg, 0.38 mmol) in a mixture of MeCN (1 mL) and H₂O (0.2 mL) was stirred at rt overnight The reaction mixture was concentrated under reduced pressure and the residue was partitioned between DCM (2 mL) and 1 M HCl (aq, 2 mL). The aqueous phase was extracted with another portion of DCM (2 mL) and the combined organic layer was concentrated under reduced pressure to give the title compound (122 mg, 96%) as a white solid; HRMS (ESI) m/z [M+H]⁺ calcd for C₃₃H₃₂Cl₂N₇O₅: 676.1836, found: 676.1866; ¹H NMR (500 MHz, CD₃OD) δ 2.04 (s, 3H), 2.74-2.87 (m, 1H), 3.05-3.20 (m, 1H), 3.26-3.29 (m, 1H), 3.33-3.45 (m, 2H), 3.49 (t, 1H), 3.73-3.81 (m, 2H), 3.95 (dd, 1H), 4.09 (d, 1H), 4.24 (dd, 1H), 4.62-4.74 (m, 2H), 4.74-4.84 (m, 2H), 4.94-5.04 (m, 1H), 6.16 (t, 1H), 6.46 (dd, 1H), 6.56 (dd, 1H), 6.77 (t, 1H), 7.28 (d, 1H), 7.48 (d, 1H), 7.66 (dd, 1H), 7.79-7.87 (m, 1H), 7.89 (dd, 1H), 7.96 (d, 1H), 8.61 (dd, 1H).

### Example 28

### 2-(((4aS,7aR)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid Isomer 1

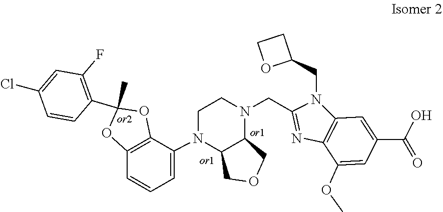 [SMILES: COc1cc(C(=O)O)cc2c1nc(CN1CCN(c3cccc4c3O[C@@](C)(c3ccc(Cl)cc3F)O4)[C@@H]3COC[C@@H]31)n2C[C@@H]1CCO1]

The stereoisomers of 2-(((4aS,7aR)-4-(2-(4-chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid Isomer 1 Intermediate 123 (700 mg, 1.03 mmol) were separated by chiral chromatography on a CHIRAL ART Cellulose-SA column (250×20 mm ID, 5 μm) eluted with 5% IPA in hexane:DCM (3:1, 0.1% FA) at a flow rate of 20 mL/min and detected at 220 and 254 nm; the first eluted compound was collected and evaporated to give the title compound Isomer 1 (148 mg, 21%) as a white solid; HRMS (ESI) m/z [M+H]⁺ calcd for C₃₅H₃₇ClFN₄O₇: 679.2330, found: 679.2372; ¹H NMR (400 MHz, DMSO-d₆) δ 7.81 (s, 1H), 7.60-7.49 (m, 2H), 7.38-7.24 (m, 2H), 6.73 (t, 1H), 6.55 (d, 1H), 6.39 (d, 1H), 4.71 (d, 1H), 4.56 (q, 1H), 4.34 (dd, 2H), 4.23 (q, 1H), 4.14 (d, 1H), 3.94 (s, 3H), 3.86-3.65 (m, 2H), 3.64-3.53 (m, 3H), 3.32 (t, 2H), 3.08 (d, 3H), 2.78-2.63 (m, 1H), 2.40 (t, 1H), 2.03 (s, 3H), 1.98 (dd, 1H), 1.73 (q, 1H), 1.52 (dq, 1H).

### Pharmacological Activity

CHOK1 GLP-1R cAMP Assay

A cell line stably expressing the human GLP-1R receptor (NM_002062.5, including the naturally-occurring variant Leu260Phe) in a CHO-K1 (ATCC® CCL-61™) was used for assay.

GLP-1 Receptor mediated agonist activity was determined in a cell based assay measuring cAMP levels in cells using Homogeneous Time-Resolved Fluorescence (HTRF) cAMP detection kit (CisBio catalog #62AM4PEC, cAMP Gs Dynamic range kit). The cAMP detection method is based on a competitive immunoassay, in which cAMP produced by the cells and cAMP labeled with the dye d2 compete for binding to a Europium-Cryptate-labeled anti-cAMP antibody. The specific HTRF signal is inversely proportional to the concentration of cAMP.

Compounds were added to individual well in 384 well-assay plates (Greiner #784076) using an Echo (LabCyte) dispenser from 10 mM stocks. Varying concentration of compounds were added to wells, and dimethyl sulfoxide was used to normalize each well to a volume of 100 nanoliter. A dose response curve of GLP1(7-36)NH2 (Bachem H-6795) was included in each run. 5 μL of cAMP concentration response standards are applied in specified wells in the assay plates.

Cryo-preserved cells are thawed and resuspended in assay buffer pre-heated to 37° C. (20 mM 2-[4-(2-hydroxyethyl)piperazin-1-yl]ethanesulfonic acid (HEPES) pH 7.4, lx Hank's Balanced Salt Solution (HBSS, Life Technologies #14065) supplemented with 0.1% (w/v) bovine serum albumin (Sigma, A-7030)). Cells were centrifuged at 250*g for 5 min at room temperature, and resuspend in room tempered assay buffer to a final density of 0.16*10⁶ cell/mL, to deliver 800 cells/well. 5 μL of assay buffer with 1 mM 3-isobutyl-1-methylxanthin (IBMX; Sigma cat I-7018) was dispensed per well in assay plates using a multidrop combi (Thermo Scientific) subsequently 5 μL of cell suspension was distributed to relevant wells in the assay using a multidrop dispenser. Assay plates were incubated 20 min at room temperature.

Detection reagents, Europium-Cryptate-labeled anti-cAMP antibody and cAMP labeled with the dye d2, are diluted in lysis buffer, provided by the manufacturer. 5 μL of each detection reagent is supplemented to each assay well using a multidrop dispenser. Assay plates are incubated in the dark for at least one hour. The HTRF signal is measured using the HTRF module (excitation: 337 nm, emission A: 665 nm and emission B: 620 nm) in Pherastar FSX (BMG Labtech).

Raw data were converted to pM cAMP using the cAMP standard curve included in each run. Converted data were further analyzed in Genedata Screener (Genedata) and EC50 determinations were made from agonist dose-response curves analyzed with a curve fitting program using a 4-parameter logistic dose response equation (Equation y=A+((B−A)/1+((C/x)\hat{ }D))) where A is no stimulation, B is full stimulation, C is the EC50 and D is the Hill slope). The percent effect was determined relative to a saturating concentration of a full GLP-1R agonist (GLP1(7-36)NH2 has 100% effect in this assay setup).

The GLP-1R EC₅₀ values for the Example compounds are set forth in Table 1 herein below.

TABLE 1

| Ex. No. | GLP-1R EC50 (nM) |
|---|---|
| 1a | 140 |
| 1b | 1.8 |
| 2a | 3.8 |
| 2b | 110 |
| 3a | 1.2 |
| 3b | 61 |
| 3c | 150 |
| 4a | 5.1 |
| 4b | 80 |
| 5a | 180 |
| 5b | 2.2 |
| 5c | 100 |
| 5d | 330 |
| 6a | 1.5 |
| 6b | 54 |
| 6c | 39 |
| 6d | 520 |
| 7a | 46 |
| 7b | 380 |
| 7c | 110 |
| 8a | 15 |
| 8b | 530 |
| 9a | 1.5 |
| 9b | 180 |
| 9c | 570 |
| 9d | 320 |
| 10 | 160 |
| 11 | 96 |
| 12a | 8 |
| 12b | 61 |
| 13a | 55 |
| 13b | 680 |
| 13c | 730 |
| 14a | 1.5 |
| 14b | 170 |
| 14c | 180 |
| 14d | 190 |
| 15a | 1.1 |
| 15b | 71 |
| 15c | 150 |
| 15d | 190 |
| 16a | 1.2 |
| 16b | 120 |
| 16c | 170 |
| 16d | 160 |
| 17 | 14 |
| 18 | 26 |
| 19 | 25 |
| 20a | 150 |
| 20b | 7.2 |
| 21 | 3 |
| 22a | 2.7 |
| 22b | 170 |
| 22c | 140 |
| 23a | 1.8 |
| 23b | 77 |
| 24 | 3.1 |
| 25 | 5.1 |
| 26 | 3.6 |
| 27 | 81 |
| 28 | 2 |

EndoC cAMP Accumulation Assay

A HTRF (Homogeneous Time-Resolved Fluorescence) cAMP assay (cAMP Gs dynamic kit; CisoBio Cat #62AM4PEJ) was used to identify agonists of the endogenous human glucagon-like peptide 1 receptor (GLP-1R) in a pancreatic insulinoma cell line (EndoC-βH1). The EndoC-βH1 cell line was sourced from Univercell Biosolutions and is a genetically engineered human pancreatic β cell line which exhibits glucose-inducible insulin secretion. EndoC-βH1 cells have detectable GLP-1R mRNA as detected by qPCR¹. The functionality of GLP-1R signalling in EndoC-βH1 has been demonstrated by Exendin-4 treatment leading to augmented insulin secretion; an effect which is blunted with shRNA-mediated knockdown of GLP-1R. The EndoC-βH1 cell line is a valid model of human beta cells and applicable for screenings to identify novel drug target candidates (Mol. Metab., 2018, 8, 144-157). CisBio HTRF cAMP kits are based on a competitive immunoassay using cryptate-labelled anti-cAMP antibody and d2-labeled cAMP. The detection kit is intended for the direct quantitative determination of cAMP. The specific signal (i.e. energy transfer) is inversely proportional to the concentration of cAMP in the standard or sample. Test compounds (10 mM in DMSO) were diluted into assay buffer (HBSS (Sigma #H8264) supplemented with 25 mM HEPES (Gibco #15630, pH 7.4), 0.1% BSA (Sigma #A3059) and 0.5 mM IBMX (Sigma #I7018) included fresh on the day of the assay) into 96 well U-bottom plates (Greiner #650201). Diluted compounds were transferred to ECHO source PP plates (Labcyte #P-05525) and dose response curves were dispensed acoustically using ECHO 550 into black shallow-well u-bottom 384-well HTRF Assay Plates (Corning 4514).

Cryovials of EndoC-H1 (supplied at 1×10e⁷ cells/vial) were used directly for screening. The cryovials and were removed from LN2 and thawed rapidly in a 37° C. water bath. The cells were resuspended in assay buffer and centrifuged at 300 g for 5 min. Cells were resuspended in assay buffer at the appropriate concentration, typically at 12e5 cells per mL (3000 cells per well, dependent on cell batch) and 2.5 μL diluted cells were added to all wells of destination plate by Multidrop combi reagent dispenser (Thermofisher). The plates were incubated at room temperature for 30 min. The assay was stopped by adding 2.5 μL anti-cAMP cryptate solution to all wells and 2.5 μL cAMP-d2 solution (both diluted 1:20 in lysis buffer) to columns 1-22 by Combi drop. A volume of 2.5 μL cAMP-d2 solution was added to wells E23 to P24 and 2.5 μL lysis buffer added to wells A23 to D24 by multichannel pipette. The plates were incubated at room temperature for 1 hr and read on an Envision plate reader using excitation wavelength of 320 nm and emission of 590 nm and 660 nm.

Raw data from Envision is converted to % DeltaF according to the manufacturer's instructions. Dose response curves are analysed via 4-Parameter Logistical Analysis and assay plate Z′ values obtained. Samples are graphed as percentage (%) activation plots compared to GIP (1-42, Bachem H-5645) with assay window defined by negative control as basal cell cAMP levels and positive control are defined by maximum GIP (82.5 nM) signal. GLP1 (7-36 amide, Bachem H-6795) dose response curve was included on all plates.

The EndoC EC₅₀ values for the Example compounds are set forth in Table 2 herein below.

TABLE 2

| Ex. No. | EndoC EC50 (nM) |
|---|---|
| 1a | 210 |
| 1b | 3.1 |
| 2a | 3.1 |
| 2b | 86 |
| 3a | 1.7 |
| 3b | 270 |
| 3c | 130 |
| 4a | 4.6 |
| 4b | 130 |
| 5a | 1300 |
| 5b | 9.5 |
| 5c | 610 |
| 5d | 2100 |
| 6a | 0.92 |
| 6b | 300 |
| 6c | 150 |
| 6d | 1100 |
| 7a | 19 |
| 7b | |
| 7c | 3900 |
| 8a | 17 |
| 8b | 1100 |
| 9a | 3.9 |
| 9b | 660 |
| 9c | 1100 |
| 9d | 2100 |
| 10 | 76 |
| 11 | 35 |
| 12a | 7.6 |
| 12b | 91 |
| 13a | 13 |
| 13b | 290 |
| 13c | 340 |
| 14a | 3.3 |
| 14b | 600 |
| 14c | 230 |
| 14d | 480 |
| 15a | 1.9 |
| 15b | 120 |
| 15c | 560 |
| 15d | 160 |
| 16a | 2.3 |
| 16b | 270 |
| 16c | 440 |
| 16d | 270 |
| 17 | 4.5 |
| 18 | 6.1 |
| 19 | 7.4 |
| 20a | 410 |
| 20b | 10 |
| 21 | 3 |
| 22a | 3.2 |
| 22b | 240 |
| 22c | 580 |
| 23a | 5 |
| 23b | 190 |
| 24 | 4.3 |
| 25 | 8 |
| 26 | 11 |
| 27 | 20 |
| 28 | 1.2 |

Inhibition of PDE3 has been shown to result in an increase in cardiovascular mortality in clinical trials (Movsesian M. A., Kukreja R. C. (2011) Phosphodiesterase Inhibition in Heart Failure. In: Francis S., Conti M., Houslay M. (eds) Phosphodiesterases as Drug Targets. Handbook of Experimental Pharmacology, vol 204. Springer, Berlin, Heidelberg. https://doi.org/10.1007/978-3-642-17969-3_10). Chronic treatment with PDE3 inhibitors has been shown to result in increased mortality, primarily as a result of arrhythmias and sudden death (Expert Opinion on Investigational Drugs, 2002, 11, 1529-1536; J. of Cardiovasc. Trans. Res., 2010, 3, 507-515) and it may therefore be an advantage to as far as possible avoid PDE3 inhibitory activity.

### PDE3 Assay

Evaluation of the effects of compounds on the activity of the human phosphodiesterase-3A is quantified by measuring the formation of 5′AMP from cAMP using a human recombinant enzyme expressed in Sf9 cells.

The test compound, reference compound or water (control) are added to a buffer containing 40 mM Tris/HCl (pH 7.4) and 8 mM MgCl₂, 450 nM cAMP and 0.25 μCi [³H]cAMP.

Thereafter, the reaction is initiated by addition of the enzyme (about 1 U) and the mixture is incubated for 20 min at 22° C.

For basal control measurements, the enzyme is omitted from the reaction mixture.

Following incubation SPA beads are added. After 30 min at 22° C. under shaking, the amount of [³H]5′AMP is quantified with a scintillation counter (Topcount, Packard).

The results are expressed as a percent inhibition of the control enzyme activity. The standard inhibitory reference compound is milrinone (CAS number 78415-72-2), which is tested in each experiment at several concentrations to obtain an inhibition curve from which its IC₅₀ value is calculated.

The PDE3 IC₅₀ values for Example compounds and reference compounds are set forth in Table 3 herein below.

TABLE 3

| Ex. No. | PDE3 IC50 (μM) |
|---|---|
| 1b | >100 |
| 2a | >100 |
| 5b | >100 |
| 6a | >100 |
| 8a | >100 |
| 9a | >100 |
| 9d | >100 |
| 21 | >100 |
| 22a | >100 |
| 23a | >100 |
| 25 | >100 |
| Ref Comp A* | 4 |
| Ref Comp B** | 11 |
| Ref Comp C*** | 2 |
| Ref Comp D**** | 5 |

*Ref Comp A may be prepared as disclosed in WO2020103815, Ex 19
**Ref Comp B may be prepared as disclosed in WO2018109607, Ex 4A-01
***Ref Comp C may be prepared as disclosed in WO2021112538, Ex 73 or as disclosed in WO2021081207, Ex 67, or as disclosed in WO2020263695, Ex 3.
***Ref Comp D may be prepared as disclosed in WO2020263695, Ex 2

1. A compound of formula (I) 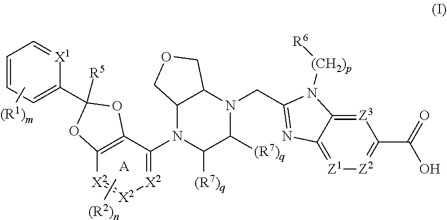
wherein
X¹ is N or C;
X² is independently N or C, provided that no more than two atoms in the aromatic ring A are N;
Z¹ is N or CR³;
Z² and Z³ are each independently N or CR⁴, provided that when Z¹ or Z³ is N, Z² is CR⁴;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
R⁷ is independently selected from F, C₁₋₂alkyl and OC₁₋₂alkyl, wherein said C₁₋₂alkyl is substituted by 0, 1, 2 or 3 substituents independently selected from F;
m is 0, 1, 2 or 3;
n is 0 or 1;
p is 1, 2 or 3;
q is 0, 1 or 2;
or a pharmaceutically acceptable salt thereof.

2. A compound according to claim 1 of Formula (Ia) 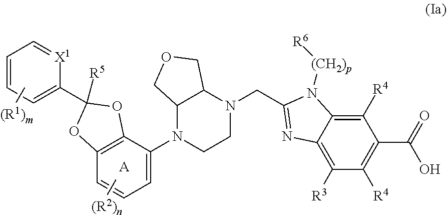
wherein
X¹ is N or C;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1, 2 or 3;
n is 0 or 1;
p is 1, 2 or 3;
or a pharmaceutically acceptable salt thereof.

3. A compound according to claim 2,
wherein
X¹ is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1 or 2;
n is 0 or 1;
p is 1, 2 or 3;
or a pharmaceutically acceptable salt thereof.

4. A compound according to claim 1 of Formula (Ic), 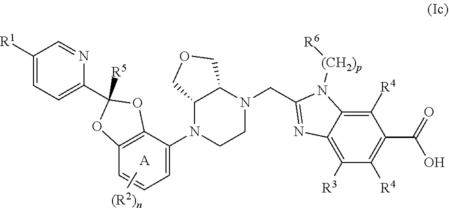
wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

5. A compound according to claim 4,
wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl;
n is 0 or 1;
p is 1;
or a pharmaceutically acceptable salt thereof.

6. A compound according to claim 1 selected from:
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
rel-1-(2-(1H-Pyrazol-1-yl)ethyl)-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
rel-1-(2-(1H-Pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
rel-1-(2-(1H-Pyrazol-1-yl)ethyl)-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylic acid,
2-(((4aR*,7aS*)-4-((R*)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylic acid,
rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid,
4-Chloro-2-(((4aR*,7aS*)-4-((R*)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
4-Chloro-2-(((4aR*,7aS*)-4-((R*)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR*,7aS*)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylic acid,
rel-4-Chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylic acid,
rel-4-Chloro-2-(((4aR,7aS)-4-((R)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylic acid,
rel-2-(((4aR,7aS)-4-((R)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylic acid,
rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylic acid,
rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid,
rel-4-Chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
rel-4-Chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-2-(((4aR,7aS)-4-((R)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
rel-1-(2-(1H-Pyrazol-1-yl)ethyl)-4-chloro-2-(((4aR,7aS)-4-((R)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
2-(((4aS,7aR)-4-((R*)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
or a pharmaceutically acceptable salt thereof.

7. A compound according to claim 1 selected from:
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
4-Chloro-2-(((4aR,7aS)-4-((s)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
4-Chloro-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
1-(2-(1H-Pyrazol-1-yl)ethyl)-2-(((4aR,7aS)-4-((S)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid,
1-(2-(1H-Pyrazol-1-yl)ethyl)-2-(((4aS,7aR)-4-((S)-2-(4-chloro-2-fluorophenyl)-2-methylbenzo[d][1,3]dioxol-4-yl)hexahydrofuro[3,4-b]pyrazin-1(2H)-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-(((S)-tetrahydrofuran-2-yl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
1-(2-(1H-Pyrazol-1-yl)ethyl)-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
1-(2-(1H-Pyrazol-1-yl)ethyl)-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
1-(2-(1H-Pyrazol-1-yl)ethyl)-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylic acid,
1-(2-(1H-Pyrazol-1-yl)ethyl)-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-4-fluoro-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-fluoro-1-(((S)-tetrahydrofuran-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(pyridin-3-ylmethyl)-1H-benzo[d]imidazole-6-carboxylic acid,
4-Chloro-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
4-Chloro-2-(((4aS,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
4-Chloro-2-(((4aR,7aS)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
4-Chloro-2-(((4aS,7aR)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(((S)-oxetan-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(((S)-oxetan-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(2-(2-methyl-1H-imidazol-1-yl)ethyl)-1H-benzo[d]imidazole-6-carboxylic acid,
4-Chloro-2-(((4aR,7aS)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylic acid,
4-Chloro-2-(((4aS,7aR)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(2-(2-methyl-1H-imidazol-1-yl)ethyl)-1H-benzo[d]imidazole-6-carboxylic acid,
4-Chloro-2-(((4aR,7aS)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylic acid,
4-Chloro-2-(((4aS,7aR)-4-((S)-2-(4-chloro-2-fluorophenyl)-2-methylbenzo[d][1,3]dioxol-4-yl)hexahydrofuro[3,4-b]pyrazin-1(2H)-yl)methyl)-1-(oxazol-4-ylmethyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-fluoro-1-(oxazol-4-ylmethyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-4-fluoro-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-4-fluoro-1H-benzo[d]imidazole-6-carboxylic acid,
4-Chloro-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
4-Chloro-2-(((4aS,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
rel-4-Chloro-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-4-Chloro-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
rel-2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-(cyanomethyl)cyclopropyl)methyl)-1H-benzo[d]imidazole-6-carboxylic acid,
2-(((4aR,7aS)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-1-((1-cyanocyclopropyl)methyl)-1H-benzo[d]imidazole-6-carboxylic acid,
1-(2-(1H-Pyrazol-1-yl)ethyl)-4-chloro-2-(((4aR,7aS)-4-((S)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
1-(2-(1H-Pyrazol-1-yl)ethyl)-4-chloro-2-(((4aS,7aR)-4-((S)-2-(5-chloropyridin-2-yl)-2-methylbenzo[d][1,3]dioxol-4-yl)hexahydrofuro[3,4-b]pyrazin-1(2H)-yl)methyl)-1H-benzo[d]imidazole-6-carboxylic acid,
2-(((4aS,7aR)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
2-(((4aR,7aS)-4-((S)-2-(4-Chloro-2-fluorophenyl)-2-methylbenzo[d][1,3]dioxol-4-yl)hexahydrofuro[3,4-b]pyrazin-1(2H)-yl)methyl)-4-methoxy-1-(((S)-tetrahydrofuran-2-yl)methyl)-1H-benzo[d]imidazole-6-carboxylic acid,
or a pharmaceutically acceptable salt thereof.

8. A compound according to claim 1 selected from:
2-(((4aS,7aR)-4-((S)-2-(5-Chloropyridin-2-yl)-2-methylbenzo[d][1,3]dioxol-4-yl)hexahydrofuro[3,4-b]pyrazin-1(2H)-yl)methyl)-4-methoxy-1-(((S)-oxetan-2-yl)methyl)-1H-benzo[d]imidazole-6-carboxylic acid,
or a pharmaceutically acceptable salt thereof.

9. A compound according to any of claims 1-8, or a pharmaceutically acceptable salt thereof, for use as a medicament.

10. A pharmaceutical composition comprising a compound according to any one of claims 1-8, or a pharmaceutically acceptable salts thereof, optionally in admixture with a pharmaceutically acceptable adjuvant, diluents or carrier.

11. A method of treating, or reducing the risk of, cardiovascular disease or metabolic conditions which comprises administering to a person suffering from or at risk of, said disease or condition, a therapeutically effective amount of a compound according to any one of claims 1-8, or a pharmaceutically acceptable salt thereof.

12. The method according to claim 11, wherein said disease is type 2 diabetes.

13. A compound according to any one of claims 1-8, or a pharmaceutically acceptable salt thereof, for use in the treatment of type 2 diabetes.

14. The use of a compound according to any one of claims 1-8, or a pharmaceutically acceptable salt thereof, in the manufacture of a medicament, for the treatment or prophylaxis of cardiovascular disease or metabolic conditions.
